(19) United States
(12) Patent Application Publication
(10) Pub. No.: US 2011/0190166 A1
(43) Pub. Date: Aug. 4, 2011

(54) ERYTHROID PROGENITOR CELLS AND METHODS FOR PRODUCING PARVOVIRUS B19 THEREIN

(76) Inventors: Susan Wong, Columbia, MD (US); Neal S. Young, Washington, DC (US); Ning Zhi, Rockville, MD (US); Kevin Brown, Kensington, MD (US)

(21) Appl. No.: 12/301,960

(22) PCT Filed: May 25, 2007
(86) PCT No.: PCT/US2007/012645

§ 371 Date: Nov. 21, 2008

Related U.S. Application Data
(60) Provisional application No. 60/808,904, filed on May 26, 2006.

(51) Int. Cl. C12N 7/00; C12Q 1/70; C12N 5/0789; C40B 40/00

(52) U.S. Cl. 506/13; 435/235.1; 435/5; 435/325

(57) ABSTRACT

The disclosure relates to erythroid progenitor cells and methods for producing parvovirus B 19 in the cells. The invention includes transformed and/or immortalized CD36+ erythroid progenitor cells permissive for B19 infection and methods for producing useful quantities of B 19 in the cells described herein. Infectious virus produced by the cells of the disclosure is useful for identifying and developing therapeutically effective compositions for treatment and/or prevention of human parvovirus B 19 infections.

### CROSS REFERENCE TO RELATED APPLICATIONS

This application is being filed on 25 May 2007, as a PCT International Patent application in the name of The Government of the United States of America as represented by the Secretary, Department of Health and Human Services, applicant for the designation of all countries except the US, and Susan Wong, Neal S. Young, citizens of the U.S., Ning Zhi, a citizen of China, and Kevin Brown, a citizen of the United Kingdom, applicants for the designation of the US only, and claims priority to U.S. Application Ser. No. 60/808,904, filed May 26, 2006, which application is incorporated by reference.

### STATEMENT OF RIGHTS TO INVENTIONS MADE UNDER FEDERALLY SPONSORED RESEARCH AND DEVELOPMENT

Part of the work performed during the development of this invention utilized United States government funds under the Division of Intramural Research, National Heart, Lung and Blood Institute.

### REFERENCE TO A CD LISTING APPENDIX

Three Compact Disc-Recordable (CD-Rs) are provided with this patent document. The CD-Rs are formatted in IBM-PC format and are compatible with the MS-Windows operating system. Each CD-R includes the following with the noted creation date: Sequence Listing (SEQ ID NOs:1-322) (dated May 25, 2007; size: 1,275 kilobytes).

The content of these files are incorporated by reference herein. The files on each CD-R are accessible using a text-based editor.

### BACKGROUND OF THE INVENTION

Human parvovirus B19 (B19) is the only member of the Parvoviridae family known to cause diseases in humans. B19 infection causes fifth disease in children, polyarthropathy syndromes in adults, transient aplastic crisis in patients with underlying chronic hemolytic anemia, and chronic anemia due to persistent infection in immunocompromised patients. Hydrops fetalis and fetal death have been reported after maternal infection with B19 during pregnancy (Brown et al., 1994, Crit. Rev. Oncol./Hematol., 16:1-13).

B19 exhibits a selective tropism for erythroid progenitor cells. The virus can be cultured in primary erythroid progenitor cells from bone marrow or from fetal liver, and cell lines such as UT7/Epo or KU812Ep6. (Ozawa et al., 1986, Science 233:883-886; Brown et al., 1991, J. Gen. Vir., 72:741-745; Komatsu et al., 1993, Blood 82:456-464; Shimomura et al., 1992, Blood 79:18-24; Miyagawa et al., 1999, J. Virol. Methods 83:45-54). Although these cells can be infected, very little virus is produced. The selective tropism of the virus is mediated in part by neutral glycolipid globoside (blood group P antigen), which is present on primary cells of the erythroid lineage (Brown et al., 1993, Science, 262:114-117). The presence of globoside on the surface of a cell is a determinant of viral tropism. B19 has a cytotoxic effect on primary erythroid progenitor cells in bone barrow and causes interruption of erythrocyte production. Human bone marrow cells that lack globoside on the cell surface are resistant to parvovirus B19 infection (Brown et al., 1994, N. Engl. J. Med., 33:1192-1196).

Currently, the most reliable source of large amounts of B19 is phlebotomy of viremic donors. Cells and methods for consistently producing infectious B19 in a significant quantity in cell culture are limited. Thus, there remains a need to develop cells capable of producing useful amounts of B19, particularly infectious B19. Infectious virus is useful for identifying and developing therapeutically effective compositions for treatment and/or prevention of human parvovirus B19 infections, such as for example, antibodies, attenuated vaccines, and chimeric viral capsid proteins comprising antigenic epitopes.

### SUMMARY OF THE INVENTION

One aspect of the invention is directed to methods of producing parvovirus B19. Virus produced by the methods of the invention is useful for identifying and developing therapeutically effective compositions for treatment and/or prevention of human parvovirus B19 infections.

The methods of producing parvovirus B19 generally include introducing a parvovirus B19 genome into a CD36⁺ erythroid progenitor cell and culturing the cell under conditions to provide for replication of parvovirus B19 genome. In some embodiments, parvovirus B19 can be introduced into CD36⁺ erythroid progenitor cells by contacting the cells with parvovirus B19 isolated from serum. In some embodiments, parvovirus B19 can be introduced into the CD36⁺ erythroid progenitor cells with a vector encoding an infectious clone of parvovirus B19 into the cells. In an embodiment, infectious clone includes a nucleic acid sequence having at least 90% nucleic acid identity to SEQ ID NO:1 or SEQ ID NO:2.

The erythroid progenitor cells, which are termed CD36⁺, can be produced from hematopoietic stem cells expressing cell surface markers such as CD34 and/or CD133 by culturing the cells in expansion media comprising stem cell factor (SCF), interleukin 3 (IL-3), and erythropoietin under conditions that allow for expansion and differentiation of the cells to a population of cells having at least 25 to 100% CD36⁺ cells. In some embodiments, expansion media comprises stem cell factor (SCF), interleukin 3 (IL-3), hydrocortisone, and erythropoietin in amounts that allow for expansion and differentiation of the cells to a population of cells having at least 25 to 100% CD36⁺ cells. In an embodiment, the expansion media comprises 5 ng/ml IL-3, 100 ng/ml recombinant human SCF, and 3 IU/ml recombinant human erythropoietin. In an embodiment, the expansion media comprises 1 nM hydrocortisone, 5 ng/ml IL-3, 100 ng/ml recombinant human SCF, and 3 IU/ml recombinant human erythropoietin. The expansion medium can have ranges of the growth factors as have been described in the art. In some embodiments, the erythroid progenitor cells are frozen, thawed, and cultured in expansion medium.

The hematopoietic stem cells are selected from a variety of source tissues for the presence of a cell surface marker such as CD34 and/or CD133. Some hematopoietic stem cells have both CD34 and CD133 on the cell surface. The source tissues include cord blood, G-CSF mobilized stem cells (or termed peripheral blood stem cells, “PBSC”), bone marrow, peripheral blood, embryonic tissue, and fetal tissue. The hematopoietic stem cells are cultured in the expansion media for about 4 days under conditions that allow for expansion and differentiation of the cells, diluted in expansion media, and the diluted cells are cultured for about an additional 4 days under conditions that allow for expansion and differentiation of the cells.

In some embodiments, the CD36 erythroid progenitor cells comprise globoside and are non-enucleated. In other embodiments, the CD36⁺ erythroid progenitor cells further comprise hemoglobin. The CD36⁺ erythroid progenitor cells comprise at least one of the following characteristics selected from the group consisting of non-enucleated; CD44⁺, CD34⁻, CD19⁻, CD10⁻, CD4⁻, CD3⁻, CD2⁻, hemoglobin; globoside; or a combination thereof. In some embodiments, the CD36⁺ erythroid progenitor cells are CD36⁺, CD44⁺, CD235a⁺, CD34⁻, CD19⁻, CD10⁻, CD4⁻, CD3⁻, and CDT. In some embodiments, the erythroid progenitor cell population has about the same percentage of cells that are CD36+ and globoside+. In some embodiments, the population has at least 25% of the cells positive for globoside and CD36. In some embodiments, the population has at least 60% of the cells positive for globoside and CD36. In some embodiments, at least 25% to 100% of the erythroid progenitor cells are CD36+ and globoside+cells, and less than 70% of the cell population are CD33+. In some embodiments, the population has at least 60% of the cells positive for globoside and CD36 and at least 50% cells positive for glycophorin by day 8 in culture.

The methods of the disclosure also include detecting reproduction of the parvovirus B19 viral transcripts, viral genome, and viral products. In some embodiments, production of the parvovirus B19 viral transcripts are detected by detecting B19 spliced capsid transcripts, unspliced capsid or NS protein transcripts or other B19 viral transcripts in the infected cells. In some embodiments, B19 capsid protein is detected by binding to a specific antibody for B19 such as an antibody for the B19 capsid protein. In some embodiments, B19 viral transcripts is detected using reverse transcription PCR (RT-PCR) or by quantitative reverse transcription PCR (qRT-PCR). In other embodiments, erythroid progenitor cells infected with B19 are detected by cytopathology and are detected as giant pronormoblasts (also described as lantern cells). One or more of these techniques may be used in conjunction with one another to confirm B19 infection.

Reproduction of the parvovirus B19 can also be detected by detecting B19 viral DNA production in the infected cells. Preferably, replication of the parvovirus B19 viral genome in the CD36⁺ erythroid progenitor cells is greater than replication of the viral genome in UT7/Epo-S1 cells. In an embodiment, replication of the parvovirus B19 viral genome in the CD36⁺ erythroid progenitor cells is at least 10 fold greater compared to UT7/Epo-S1 cells. In another embodiment, replication of the parvovirus B19 viral genome in the CD36⁺ erythroid progenitor cells is at least 100 fold greater compared to UT7/Epo-S1 cells. In yet another embodiment, replication of the parvovirus B19 viral genome in the CD36⁺ erythroid progenitor cells is at least 500 fold greater compared to UT7/Epo-S1 cells. Preferably, parvovirus B19 production is greater in CD36⁺ erythroid progenitor cells compared to UT7/Epo-S1 cells. In an embodiment, parvovirus B19 production in CD36⁺ erythroid progenitor cells is increased at least 1.5 log compared to UT7/Epo-S1 cells. Preferably, the replicated parvovirus B19 is infectious.

Detection of infectious B19 virus can be assessed by the presences of B19 DNA by in vitro assays such as PCR but the presence of B19 DNA is not necessarily indicative of the presence of infectious virus. The presence of infectious virus can be determined by an in vitro bioassay using B19 containing material to infect CD36+ cells. In this case, a DNA increase or RNA production would indicate the presence of infectious virus

Reproduction of infectious parvovirus B19 in infected CD36⁺ erythroid progenitor cells can also be detected by contacting uninfected permissive cells with supernatant from the infected CD36⁺ erythroid progenitor cells and analyzing the contacted permissive cells for B19 viral transcripts, B19 viral proteins, or increase viral DNA production. Detection of B19 viral transcripts, B19 viral proteins, or increase viral DNA production in the contacted permissive cells indicates that the parvovirus B19 is infectious. The permissive cells can be erythroid progenitor cells found in bone marrow or fetal liver, UT7/Epo cells, UT7/Epo-S1 cells, or KU812Ep6 cells. In an embodiment, the permissive cells are erythroid progenitor cells that are CD36⁺, CD44⁺, CD235a⁺, CD34⁻, CD19⁻, CD10⁻, CD4⁻, CD3⁻, CD2⁻, and globoside positive.

In another aspect, the disclosure also provides a cell population comprising erythroid progenitor cells, wherein at least 25% to 100% of the erythroid progenitor cells are CD36+ and globoside+cells, and less than 70% of the cell population are CD33+. In other embodiments, CD36+ erythroid progenitor cells are produced by a method comprising: culturing hematopoietic stem cells in expansion media comprising stem cell factor (SCF), interleukin 3 (IL-3), and erythropoietin under conditions that allow for expansion and differentiation of the cells to a population of cells having at least 25% CD36⁺ cells. In an embodiment, the hematopoietic stem cells have CD34, CD133, or both on the cell surface. In some embodiments, the expansion media comprises 10⁻⁶ M hydrocortisone, 5 ng/ml, IL-3, 100 ng/ml recombinant human SCF, and 3 IU/ml recombinant human erythropoietin. In some embodiments, the hematopoietic stem cells are cultured in the expansion media for about 4 days under conditions that allow for expansion and differentiation of the cells, diluted in expansion media, and the diluted cells are cultured for about an additional 4 days under conditions that allow for expansion and differentiation of the cells. In some embodiments, the cell population or erythroid progenitor cells comprise CD36⁺ erythroid progenitor cells that are CD36⁺, CD44⁺, CD235a⁺, CD34⁻, CD19⁻, CD10⁻, CD4⁻, CD3⁻, and CDT.

Another aspect of the invention is immortalized erythroid progenitor cells that are permissive to parvovirus B19 infection and methods of making the immortalized-cells. The immortalized erythroid progenitor cells can be produced by culturing hematopoietic cells in expansion media under conditions that allow for expansion and differentiation of the cells to a population of at least 25 to 100% CD36⁺ cells; and immortalizing the CD36⁺ erythroid progenitor cells. In some embodiments, the cells can be immortalized by transforming the CD36⁺ erythroid progenitor cells with a viral vector comprising a nucleic acid encoding a SV40 large T-antigen, hTERT (human telomerase reverse transcriptase gene), and/or HPVtype 16 E6/E7. In some embodiments, the viral vector comprises adenovirus, lentivirus, retrovirus, or adeno-associated virus (AAV). In other embodiments, the cells are immortalized with Epstein Barr virus.

In some embodiments, the method for immortalizing the CD36⁺ erythroid progenitor cells includes culturing the hematopoietic stem cells in expansion media for about 4 days under conditions that allow for expansion and differentiation of the cells, diluting the cells in expansion media, and culturing the diluted cells for about 4 days under conditions that allow for expansion and differentiation of the cells. The immortalized erythroid progenitor cells comprise globoside and are non-enucleated. In some embodiments, the immortalized cells further comprise hemoglobin. The immortalized erythroid progenitor cells comprise at least one of the following characteristics selected from the group consisting of: non-enucleated; CD44⁺, CD34⁻, CD19⁻, CD10⁻, CD4⁻, CD3⁻, CDT, hemoglobin; globoside; or a combination thereof. In some embodiments, the immortalized CD36⁺ erythroid progenitor cells are globoside+, CD36⁺, CD44⁺, CD235a⁺, CD34⁻, CD19⁻, CD10⁻, CD4⁻, CD3⁻, and CDT. In some embodiments, at least 25% to 100% of the erythroid progenitor cells are CD36+ and globoside+cells, and less than 70% of the cell population are CD33+. In an embodiment, the immortalized erythroid progenitor cells can divide at least 2 to 50 times. In an embodiment, the cells are a continuous cell line that divides indefinitely.

Another aspect of the invention includes diagnostic kits and assays. The kits and assays can be used to detect, for example, antibodies to parvovirus B19. In an embodiment, the kit includes a composition comprising parvovirus B19 particles produced by the CD3+ erythroid progenitor cells or immortalized CD36⁺ erythroid progenitor cells of the invention and instructions for using the parvovirus B19 produced by the cells to detect antibodies to parvovirus B19. In other embodiments, the kits can include probes or primers for detecting the presence of viral transcripts. In a specific embodiment, viral transcripts of capsid protein and/or NS protein are detected. In other embodiments an increase in viral RNA or DNA may be detected.

In an embodiment, diagnostic kits or assays can be used to identify neutralizing antibodies. Antibodies produced against B19 may not be effectively neutralizing or partially neutralizing.

In an embodiment, diagnostic kits or assays can be used to identify infectious B19 virions. B19 has been known to produce 1 infectious particle in 10e3 to 10e5 particles. B19 DNA has also been known to persist for years after infection of an individual. In an embodiment, CD36⁺ cells allow a determination the presence of infectious virions by the production of B19 transcripts or increasing DNA production.

In other embodiments diagnostic kits and assays may also include agents for the detection of biomarkers or genes differentially expressed in B19 virus infected cells. The agents for detection include antibodies, probes, primer, and agents for assay of activity of the biomarker. Biomarkers of B19 infected cells include one or more of differentially expressed genes as shown in Table 15, comparing timepoint zero infection to any other timepoint such as 3, 6, 12, 24, and 48 hours and even up to 5 days post infection. In some embodiments, the diagnostic assay or kit may include agents for detecting at least one of 1, 2, 3, 4, 5, 6, 7, 8, 9, 10, and up to all of the 309 genes. Some of the genes differentially expressed may be detected as secreted products. In other embodiments, the genes selected are differentially (increased or decreased) expressed at least two fold at 48 hours post infection as compared to uninfected cells.

In some embodiments, a method of detecting a parvovirus B19 infection comprises contacting the CD36+ erythroid progenitor cell with a sample; culturing the cells under conditions suitable for viral replication; and detecting the presence of the virus in the cell. In some embodiments, the CD36⁺ erythroid progenitor cells are cultured in expansion media comprising stem cell factor (SCF), interleukin 3 (IL-3), and erythropoietin under conditions that allow for expansion and differentiation of the cells to a population of cells having at least 25% CD36⁺ cells. In an embodiment, the expansion media comprises 10⁻⁶M hydrocortisone, 5 ng/ml IL-3, 100 ng/ml recombinant human SCF, and 3 IU/ml recombinant human erythropoietin. In some embodiments, the CD36⁺ erythroid progenitor cells are CD36⁺, CD44⁺, CD235e, CD34⁻, CD19⁻, CD10⁻, CD4⁻, CD3⁻, CD2⁻, non-enucleated, may have hemoglobin or may have globoside and combinations thereof. In an embodiment, the population of CD36⁺ erythroid progenitor cells comprise at least 25% to 100% CD36+ cells. In an embodiment, the population of CD36⁺ erythroid progenitor cells comprise at least 25% CD36+ cells and 25% globoside positive cells. In some embodiments, at least 25% to 100% of the erythroid progenitor cells are CD36+ and globoside+cells, and less than 70% of the cell population are CD33⁺.

In other embodiments, the method further comprises detecting reproduction of the parvovirus B19 viral genome, transcripts, or viral protein. In some embodiments, detection of reproduction of the parvovirus B19 viral genome comprises detecting B19 DNA, spliced capsid transcripts, unspliced capsid or NS protein transcripts, or B19 capsid protein in the infected cells. In an embodiment, the B19 capsid protein is detected by binding to a specific antibody for B19 capsid protein. In an embodiment, the B19 transcripts are detected using RT-PCR or by qRT-PCR. In an embodiment, detection of reproduction of the parvovirus B19 viral genome comprises detecting B19 viral DNA in the cell.

In some embodiments, a method of detecting a parvovirus B19 infection comprises contacting a cell or population of CD36+ erythroid progenitor cell with a sample; culturing the cells under conditions suitable for viral replication; and detecting the gene expression profile of at least one of the genes of Table 15 and at least one parvovirus B19 viral genome, transcript, or viral protein. In some embodiments, expression of at least one or all of the genes of Table 16 are detected. In some embodiments, expression of the genes is detected at 6 and/or 48 hours post infection. In some embodiments, the gene expression is detected by an oligonucleotide that specifically binds to the polynucleotide encoding the gene.

Another aspect of the disclosure provides for kits for diagnosis of B19 infection. In an embodiment, a kit comprises a composition comprising a CD36+ erythroid progenitor cell and a composition comprising a parvovirus B19 virus sample. In an embodiment, the parvovirus B19 composition comprises at least 10³ genomes/ml of parvovirus B19. In other embodiments, a kit for detecting or diagnosing parvovirus B19 infection, comprises: a CD36+ erythroid progenitor cell as described herein, and at least one oligonucleotide that specifically binds to a) a parvovirus B19 genome, or b) at least one viral transcript and/or an antibody that specifically binds to a viral protein. In some embodiments, the kit for diagnosing or detecting, further comprises a composition comprising a parvovirus B19 virus sample. In other embodiments, a kit for detecting or diagnosing parvovirus B19 infection, comprises: a) a CD36+ erythroid progenitor cell as described herein and b) at least one oligonucleotide that specifically binds to parvovirus B19 genome or at least one viral transcript and/or an antibody that specifically binds to a viral protein; and c) at least one oligonucleotide that specifically binds to at least one of the genes of Table 15. In some embodiments, the kit for diagnosing or detecting, further comprises a composition comprising a parvovirus 1319 virus sample.

Another aspect of the disclosure provides a microarray. In some embodiments, a microarray comprises agents that bind to 400 genes or less including at least one or all of the genes of Table 15. In other embodiments, the microarry comprises agents that bind to 400 genes or less including at least one or all of the genes of Table 16. In yet another embodiment, a microarray comprises agents that bind to 400 genes or less including at least one or all of the genes of Table 16 and at least one or all of the parvovirus B19 transcripts. In some embodiments, a microarray comprises agents that bind to 400 genes or less including at least one or all parvovirus B19 transcripts or parvovirus B19 genome. Agents include oligonucleotide probes, or antibodies or antibody fragments.

Parvovirus B19 virus particles and/or clones produced by the cells or methods of the invention can be utilized to form immunogenic compositions to prepare therapeutic antibodies or vaccine components. In an embodiment, the immunogenic composition comprises parvovirus B19 particles produced by the immortalized CD36⁺ erythroid progenitor cells. The parvovirus B19 particles can be attenuated or heat killed.

### BRIEF DESCRIPTION OF THE DRAWINGS

FIG. 1. Rapid CD36⁺ proliferation in expansion medium (numbers of cells/days of culture). Three sets of purified CD34+ cells derived from G-CSF mobilized PBSC were cultured in expansion media. To determine if cells would survive and proliferate after cryopreservation, two of the sets were frozen at day 4 in culture and revived.

FIG. 2A. Comparison of cell proliferation of CD36+ day 8 cells which are uninfected and cells infected with B19. FIG. 2B. Comparison of cell proliferation of day 8 UT7/Epo-S1 uninfected cells and cells infected with B19.

FIG. 3A. Daily timecourse evaluating B19 transcript production in CD36+ cells infected with serial dilution of B19 (copies/μL). B19 NS transcript analyzed by qRT-PCR.

FIG. 3B. B19 CP (capsid) transcript analyzed by qRT-PCR.

FIG. 4. NS transcript production on day 2 post infection in CD36+ day 7 cells infected with serial dilutions of B19 containing plasma (V1). Infectious virus in viremic sample V1 can be detected to 10e2/mL which is 2-logs higher sensitivity than that detected in UT7/Epo-S1 cells.

FIG. 5A. NS transcript production during an hourly time course in UT7/Epo-S1 and CD36+ day 8 cells. Compared to B19 transcripts produced in UT7/Epo-S1 cells, CD36⁺ day 8 cells generated a 1-2 log greater amount of B19 transcripts at each timepoint. FIG. 5B. UT7/Epo-S1 and CD36+ day 8 CP transcript production during an hourly time course. A 1-2 log increase in B19 transcripts in CD36+ day 8 cells is shown at each timepoint.

FIG. 6. Comparison by qPCR analysis of CD36⁺ day 8 cells and UT7/Epo-S1 cells for viral B19 DNA production in cells infected with serial dilutions of B19 containing plasma.

FIG. 7. CD36+ cells infected with supernatants from cellular lysates from three successive rounds of infection. Initially, cells were infected with B19 containing plasma V1 and the cellular lysate was freeze-thawed three times and used to inoculate naïve cells. Similar was done for two successive passages of the virus.

FIG. 8 NS transcripts. CD36+ day 8 cells were transfected with the B19 infectious clone, pB19-M20, in two experiments, pB19-M20 (1) and (2). Transfected cells were harvested after three days (Transfection D3) and cellular lysates were used to infect naïve cells (Infection D0) and finally, the infected cells were harvested on day 3 (Infection D3.) qRT-PCR was used to analyze the transcript production at each of the timepoints.

FIG. 9 shows the increase in B19 DNA production in CD36⁺ erythroid progenitor cells that were transformed using a recombinant adenovirus containing SV40 large T antigen 3 days post infection (output) with B19 as compared to the input virus.

FIG. 10 shows B19 NS transcripts detected by qRT-PCR in adenovirus-SV40 transformed CD36⁺ erythroid progenitor cells infected with B19 at 0 to 3 days post infection. The data in FIG. 10 indicates that the transformed CD36⁺ erythroid progenitor cells infected with B19 are producing viral genomes.

FIG. 11 shows the time course of expansion of CD133+ stem cells in expansion medium.

FIG. 12A Immunofluorescence Assay of CD133⁺ selected cells cultured in expansion media for 8 days and then infected with B19. Antibody 521-5D against the B19 capsid region (bright fluorescence in figure). CD133⁺ selected PBSC expanded and differentiated in expansion media show similar sensitivity as CD34⁺ selected PBSC to B19 infection. FIG. 12B Immunofluorescence Assay of CD36+ day 8 cells transfected with pB19-M20. Antibody 521-5D against the B19 capsid region (fluorescence in figure).

FIG. 13 Differentially expressed genes involved in regulation of G1/S transition during B19 infection.

### DETAILED DESCRIPTION OF THE INVENTION

### I. Definitions

The term “parvovirus B19”, “B19”, “B19V”, “B19 virus”, “B19 clone”, “B19 isolate”, or B19 means an isolate, clone or variant 1319 viral genome of parvovirus B19 or parvovirus B19 virus particle of the family Parvoviridae including genotypes 1, 2, and 3.

“Variants” of the parvovirus B19 viral genome refer to a sequence of a viral genome that differs from a reference sequence and includes “naturally occurring” variants as well as variants that are prepared by altering of one or more nucleotides.

An “infectious clone” of parvovirus B19 as used herein refers to a full-length genome or portion of a genome of a parvovirus B19 isolate cloned into a replicable vector that provides for amplification of the viral genome in a cell. In some embodiments, a portion of the parvovirus B19 genome comprises or consists of nucleic acid sequence encoding at least one ITR, VP2, NS, and 11-kDa in a single replicable vector. In other embodiments, the viral genome is a full-length genome. The replicable vector provides for introduction and amplification of the viral genome in a wide variety of prokaryotic and eukaryotic cells, whether or not they have globoside.

The term “hematopoietic stem cell” or “hemapoeitic stem cell” as used herein refers to a precursor cell that is capable of differentiating to a red blood cell. In some embodiments, precursor cells can be isolated from sources including, but not necessarily limited to: cord blood, G-CSF mobilized stem cells (or termed peripheral blood stem cells, “PBSC”), bone marrow, peripheral blood, embryonic tissue, and fetal tissue Generally, hematopoietic stem cells may be found in a variety of sources of tissues. In some embodiments, hematopoietic stem cells derived from (but not limited to) the above sources are selected by cell surface antigens such as (but not limited to) CD34 or CD133. Other markers of hematopoietic stem cells may include CD33, CD34, CD90, CD110, CD111, CD112, CD117, CD123, CDw131, CD133, CD135, CD173, CD174, CD176, CD243, CD277, CD280, CD297, CD318, CD324, or CDw388.

The term “erythroid progenitor cell” as used herein refers to a red blood cell precursor cell that is capable of differentiating to a red blood cell.

The term “CD36⁺ cells” or “primary CD36⁺ cells,” or “CD36⁺ erythroid progenitor cells” refers to cells generated through the culture of hematopoietic stem cells or hematopoietic precursor cells grown in the defined expansion media for any given number of days after introduction into culture. Related terms defining the number of days in which the cells are in culture will be defined in the following manner: “CD36⁺ day 4 cells,” “CD36⁺ day 5 cells,” “CD36⁺ day 6 cells,” “CD36⁺ day 7 cells,” “CD36⁺ day 8 cells,” and so on.

“Secondary cell” as used herein refers to cells with an extended replicative capacity or life span in culture as compared to a primary culture of cells of the same cell type but do not continue to divide indefinitely and eventually senesce and die. In some embodiments, secondary cells can be prepared by growing the cells in specialized media which induce cells to differentiate and have characteristics different from the primary cells in which they were derived. In some embodiments, secondary cells can be prepared by transformation of primary cells with a vector comprising a polynucleotide that inactivates tumor suppressor genes in the transformed cells that results in a replicative senescent state or a polynucleotide that regulates the expression or activity of telomerase. Secondary cells can continue growth in culture from about two divisions or generations to about 100 divisions or generations. In some embodiments, secondary cells can continue growth through at least 10 to 30 divisions. In some embodiments, the doubling time of the secondary cells is from about 12 hours to about 36 hours.

“Transformed cells” as used herein refers to cells that have at least one of the growth properties selected from the group consisting of anchorage independent, loss of contact inhibition, growth in suspension, growth factor independent, shorter population doubling time, increased life span of about 2 to 50 generations and combinations thereof. In some embodiments, transformed cells refer to cells that have been infected with or transfected with a vector, including a vector comprising the SV40 large T antigen.

“Immortalized cells” as used herein refers to cells that have an increased ability to divide in vitro as long the appropriate culture conditions are maintained. In an embodiment, the cells are a continuous cell line that divides indefinitely. In some embodiments, immortalization of a cell can result in a secondary cell. In other embodiments, the immortalized cells can grow and divide indefinitely. Methods for immortalizing cells in culture are known. See, for example, Culture of Immortalized Cells, Freshney and Freshney Eds., Wiley Publishing Inc, Indianapolis, Ind., 1996 and Hahn, W C, 2002, Mol. Cells, 13:351-361. The methods include, but are not limited to, chemical mutagenesis, transforming cells with a vector comprising a polynucleotide that inactivates tumor suppressor genes in the transformed cells that results in a replicative senescent state or a polynucleotide that regulates the expression or activity of telomerase.

The term “full length genome” refers to a complete coding sequence of a viral genome that comprises at least 75% or greater of the nucleotide sequence that forms the hairpin of the ITR at the 5′ end and 3′ end of the genome.

The term “infection” as used herein refers to the attachment of B19 virus to the cellular surface of a host cell and penetrating the cells as to allow introduction of B19 viral DNA into a cell. Cells are typically infected by contacting a cell with B19 virus. Attachment of viral particles is typically facilitated by binding to a receptor on the cellular surface. Infection of a cell by B19 virus may be determined by analyzing the cell for viral RNA, viral DNA or viral protein production. Infection of a cell by 1319 virus may be determined by detecting viral transcripts, including, but not limited to, capsid protein transcripts (VP1 or VP2) and nonstructural protein (NS) transcripts. Infection of a cell by B19 virus may be determined by detection of viral proteins including but not limited to capsid proteins (VP1 or VP2) and nonstructural proteins (NS).

The term “infectious virus” as used herein refers to the ability of a virus to infect a cell. Infectious virus has the ability to interact with a cell to release the viral contents comprising of DNA, RNA and/or viral proteins into the host cell.

The term “immunogenic effective amount” of a parvovirus B19 or component of a parvovirus refers to an amount of a parvovirus B19 or component thereof that induces an immune response in an animal. The immune response may be determined by measuring a T or B cell response. Typically, the induction of an immune response is determined by the detection of antibodies specific for parvovirus B19 or component thereof.

The term “permissive cells” refers to cells that are susceptible to infection by B19. A permissive cell has appropriate receptors on its cell surface permitting viral attachment, interactions, and entry. A permissive cell infected with B19 may or may not produce infectious virus particles. In some embodiments, permissive cells are eukaryotic cells.

Examples of permissive cells include, but are not limited to primary erythroid progenitor cells from bone marrow, blood, or fetal liver, megakaryoblast cells, UT7/Epo cells, UT7/Epo-S1 cells, KU812Ep6 cells, JK-1 cells, MB-02 cells, as well as the cells described herein.

“Percent (%) nucleic acid sequence identity” with respect to the nucleic acid sequences identified herein is defined as the percentage of nucleotides in a candidate sequence that are identical with the nucleotides in a reference B19 nucleic acid sequence, after aligning the sequences and introducing gaps, if necessary, to achieve the maximum percent sequence identity. In some embodiments, the reference B19 nucleic acid sequence is that of SEQ ID NO:307 (Table 1) or that of SEQ ID NO:308 (Table 2).

TABLE 1

| | |
|---|---|
| 1 | aaatcaga tgccgccggt cgccgccggt aggcgggact tccggtacaa gatggcggac |
| 59 | aattacgtca tttcctgtga cgtcatttcc tgtgacgtca cttccggtgg gcgggacttc |
| 119 | cggaattagg gttggctctg ggccagcttg cttggggttg ccttgacact aagacaagcg |
| 179 | gcgcgccgct tgatcttagt ggcacgtcaa ccccaagcgc tggcccagag ccaaccctaa |
| 239 | ttccggaagt cccgcccacc ggaagtgacg tcacaggaaa tgacgtcaca ggaaatgacg |
| 299 | taattgtccg ccatcttgta ccggaagtcc cgcctaccgg cggcgaccgg cggcatctga |
| 359 | tttggtgtct tcttttaaat tttagcgggc ttttttcccg ccttatgcaa atgggcagcc |
| 419 | attttaagtg ttttactata attttattgg tcagttttgt aacggttaaa atgggcggag |
| 479 | cgtaggcggg gactacagta tatatagcac agcactgccg cagctctttc tttctgggct |
| 539 | gctttttcct ggactttctt gctgtttttt gtgagctaac taacaggtat ttatactact |
| 599 | tgttaatata ctaacatgga gctatttaga ggggtgcttc aagtttcttc taatgttctg |
| 659 | gactgtgcta acgataactg gtggtgctct ttactagatt tagacacttc tgactgggaa |
| 719 | ccactaactc atactaacag actaatggca atatacttaa gcagtgtggc ttctaagctt |
| 779 | gaccttaccg gggggccact agcagggtgc ttgtactttt ttcaagcaga atgtaacaaa |
| 839 | tttgaagaag gctatcatat tcatgtggtt attggggggc cagggttaaa ccccagaaac |
| 899 | ctcacagtgt gtgtagaggg gttatttaat aatgtacttt atcactttgt aactgaaaat |
| 959 | gtgaagctaa aatttttgcc aggaatgact acaaaaggca aatactttag agatggagag |
| 1019 | cagtttatag aaaactattt aatgaaaaaa atacctttaa atgttgtatg gtgtgttact |
| 1079 | aatattgatg gatatataga tacctgtatt tctgctactt ttagaagggg agcttgccat |
| 1139 | gccaagaaac cccgcattac cacagccata aatgatacta gtagcgatgc tggggagtct |
| 1199 | agcggcacag gggcagaggt tgtgccattt aatgggaagg gaactaaggc tagcataaag |
| 1259 | tttcaaacta tggtaaactg gttgtgtgaa aacagagtgt ttacagagga taagtggaaa |
| 1319 | ctagttgact ttaaccagta cactttacta agcagtagtc acagtggaag ttttcaaatt |
| 1379 | caaagtgcac taaaactagc aatttataaa gcaactaatt tagtgcctac tagcacattt |
| 1439 | ttattgcata cagactttga gcaggttatg tgtattaaag acaataaaat tgttaaattg |
| 1499 | ttactttgtc aaaactatga ccccctattg gtggggcagc atgtgttaaa gtggattgat |
| 1559 | aaaaaatgtg gcaagaaaaa tacactgtgg ttttatgggc cgccaagtac aggaaaaaca |
| 1619 | aacttggcaa tggccattgc taaaagtgtt ccagtatatg gcatggttaa ctggaataat |
| 1679 | gaaaactttc catttaatga tgtagcagga aaaagcttgg tggtctggga tgaaggtatt |
| 1739 | attaagtcta caattgtaga agctgcaaaa gccattttag gcgggcaacc caccagggta |
| 1799 | gatcaaaaaa tgcgtggaag tgtagctgtg cctggagtac ctgtggttat aaccagcaat |
| 1859 | ggtgacatta cttttgttgt aagcgggaac actacaacaa ctgtacatgc taaagcctta |
| 1919 | aaagagcgca tggtaaagtt aaactttact gtaagatgca gccctgacat ggggttacta |
| 1979 | acagaggctg atgtacaaca gtggcttaca tggtgtaatg cacaaagctg ggaccactat |
| 2039 | gaaaactggg caataaacta cacttttgat ttccctggaa ttaatgcaga tgccctccac |
| 2099 | ccagacctcc aaaccacccc aattgtcaca gacaccagta tcagcagcag tggtggtgaa |
| 2159 | agctctgaag aactcagtga aagcagcttt tttaacctca tcaccccagg cgcctggaac |
| 2219 | actgaaaccc cgcgctctag tacgcccatc cccgggacca gttcaggaga atcatttgtc |
| 2279 | ggaagcccag tttcctccga agttgtagct gcatcgtggg aagaagcctt ctacacacct |
| 2339 | ttggcagacc agtttcgtga actgttagtt ggggttgatt atgtgtggga cggtgtaagg |
| 2399 | ggtttacctg tgtgttgtgt gcaacatatt aacaatagtg ggggaggctt gggactttgt |
| 2459 | ccccattgca ttaatgtagg ggcttggtat aatggatgga aatttcgaga atttacccca |
| 2519 | gatttggtgc gatgtagctg ccatgtggga gcttctaatc ccttttctgt gctaacctgc |
| 2579 | aaaaaatgtg cttacctgtc tggattgcaa agctttgtag attatgagta aagaaagtgg |
| 2639 | caaatggtgg gaaagtgatg atgaatttgc taaagctgtg tatcagcaat ttgtggaatt |
| 2699 | ttatgaaaag gttactggaa cagacttaga gcttattcaa atattaaaag atcattataa |
| 2759 | tatttcttta gataatcccc tagaaaaccc atcctctctg tttgacttag ttgctcgcat |
| 2819 | taaaaataac cttaaaaatt ctccagactt atatagtcat cattttcaaa gtcatggaca |
| 2879 | gttatctgac cacccccatg ccttatcatc cagtagcagt catgcagaac ctagaggaga |
| 2939 | agatgcagta ttatctagtg aagacttaca caagcctggg caagttagcg tacaactacc |
| 2999 | cggtactaac tatgttgggc ctggcaatga gctacaagct gggcccccgc aaagtgctgt |
| 3059 | tgacagtgct gcaaggattc atgactttag gtatagccaa ctggctaagt tgggaataaa |
| 3119 | tccatatact cattggactg tagcagatga agagctttta aaaaatataa aaaatgaaac |
| 3179 | tgggtttcaa gcacaagtag taaaagacta ctttacttta aaaggtgcag ctgcccctgt |
| 3239 | ggcccatttt caaggaagtt tgccggaagt tcccgcttac aacgcctcag aaaaataccc |
| 3299 | aagcatgact tcagttaatt ctgcagaagc cagcactggt gcaggagggg ggggcagtaa |
| 3359 | tcctgtcaaa agcatgtgga gtgagggggc cacttttagt gccaactctg tgacttgtac |
| 3419 | attttctaga cagtttttaa ttccatatga cccagagcac cattataagg tgttttctcc |
| 3479 | cgcagcaagt agctgccaca atgccagtgg aaaggaggca aaggtttgca ccattagtcc |
| 3539 | cataatggga tactcaaccc catggagata tttagatttt aatgctttaa acttattttt |
| 3599 | ttcaccttta gagtttcagc acttaattga aaattatgga agtatagctc ctgatgcttt |
| 3659 | aactgtaacc atatcagaaa ttgctgttaa ggatgttaca gacaaaactg gagggggggt |
| 3719 | gcaggttact gacagcacta cagggcgcct atgcatgtta gtagaccatg aatacaagta |
| 3779 | cccatatgtg ttagggcaag gtcaagatac tttagcccca gaacttccta tttgggtata |
| 3839 | ctttccccct caatatgctt acttaacagt aggagatgtt aacacacaag gaatttctgg |
| 3899 | agacagcaaa aaattagcaa gtgaagaatc agcattttat gttttggaac acagttcttt |
| 3959 | tcagctttta ggtacaggag gtacagcaac tatgtcttat aagtttcctc cagtgccccc |
| 4019 | agaaaattta gagggctgca gtcaacactt ttatgagatg tacaatccct tatacggatc |
| 4079 | ccgcttaggg gttcctgaca cattaggagg tgacccaaaa tttagatctt taacacatga |
| 4139 | agaccatgca attcagcccc aaaacttcat gccagggcca ctagtaaact cagtgtctac |
| 4199 | aaaggaggga gacagctcta atactggagc tgggaaagcc ttaacaggcc ttagcacagg |
| 4259 | tacctctcaa aacactagaa tatccttacg cccggggcca gtgtctcagc cgtaccacca |
| 4319 | ctgggacaca gataaatatg tcacaggaat aaatgctatt tctcatggtc agaccactta |
| 4379 | tggtaacgct gaagacaaag agtatcagca aggagtgggt agatttccaa atgaaaaaga |
| 4439 | acagctaaaa cagttacagg gtttaaacat gcacacctac tttcccaata aaggaaccca |
| 4499 | gcaatataca gatcaaattg agcgccccct aatggtgggt tctgtatgga acagaagagc |
| 4559 | ccttcactat gaaagccagc tgtggagtaa aattccaaat ttagatgaca gttttaaaac |
| 4619 | tcagtttgca gccttaggag gatggggttt gcatcagcca cctcctcaaa tatttttaaa |
| 4679 | aatattacca caaagtgggc caattggagg tattaaatca atgggaatta ctaccttagt |
| 4739 | tcagtatgcc gtgggaatta tgacagtaac catgacattt aaattggggc cccgtaaagc |
| 4799 | tacgggacgg tggaatcctc aacctggagt atatcccccg cacgcagcag gtcatttacc |
| 4859 | atatgtacta tatgacccta cagctacaga tgcaaaacaa caccacagac atggatatga |
| 4919 | aaagcctgaa gaattgtgga cagccaaaag ccgtgtgcac ccattgtaaa cactccccac |
| 4979 | cgtgccctca gccaggatgc gtaactaaac gcccaccagt accacccaga ctgtacctgc |
| 5039 | cccctcctat acctataaga cagcctaaca caaaagatat agacaatgta gaatttaagt |
| 5099 | atttaaccag atatgaacaa catgttatta gaatgttaag attgtgtaat atgtatcaaa |
| 5159 | atttagaaaa ataaacgttt gttgtggtta aaaaattatg ttgttgcgct ttaaaaattt |
| 5219 | aaaagaagac accaaatcag atgccgccgg tcgccgccgg taggcgggac ttccggtaca |
| 5279 | agatggcgga caattacgtc atttcctgtg acgtcatttc ctgtgacgtc acttccggtg |
| 5339 | ggcggaactt ccggaattag ggttggctct gggccagcgc ttggggttga cgtgccacta |
| 5399 | agatcaagcg gcgcgccgct tgtcttagtg tcaaggcaac cccaagcaag ctggcccaga |
| 5459 | gccaacccta attccggaag tcccgcccac cggaagtgac gtcacaggaa atgacgtcac |
| 5519 | aggaaatgac gtaattgtcc gccatcttgt accggaagtc ccgcctaccg gcggcgaccg |
| 5579 | gcggcatctg attt |

TABLE 2

| | |
|---|---|
| 1 | gaattccgcc aaatcagatg ccgccggtag ccgccggtag gcgggacttc cggtacaaga |
| 61 | tggcggacaa ttacgtcatt tcctgtgacg tcatttcctg tgacgtcaca ggaaatgacg |
| 121 | taattgtccg ccatcttgta ccggaagtcc cgcctaccgg cggcgaccgg cggcatctga |
| 181 | tttggtgtct tcttttaaat tttagcgggc ttttttcccg ccttatgcaa atgggcagcc |
| 241 | attttaagtg ttttactata attttattgg ttagttttgt aacggttaaa atgggcggag |
| 301 | cgtaggcggg gactacagta tatatagcac ggtactgccg cagctctttc tttctgggct |
| 361 | gctttttcct ggactttctt gctgtttttt gtgagctaac taacaggtat ttatactact |
| 421 | tgttaacatc ctaacatgga gctatttaga ggggtgcttc aagtttcttc taatgttcta |
| 481 | gactgtgcta acgataactg gtggtgctct ttactggatt tagacacttc tgactgggaa |
| 541 | ccactaactc atactaacag actaatggca atatacttaa gcagtgtggc ttctaagctt |
| 601 | gactttaccg gggggccact agcagggtgc ttgtactttt ttcaagtaga atgtaacaaa |
| 661 | tttgaagaag gctatcatat tcatgtggtt actggggggc cagggttaaa ccccagaaac |
| 721 | cttacagtgt gtgtagaggg gttatttaat aatgtacttt atcaccttgt aactgaaaat |
| 781 | gtgaagctaa aatttttgcc aggaatgact acaaaaggca aatactttag agatggagag |
| 841 | cagtttatag aaaactattt aatgaaaaaa atacctttaa atgttgtatg gtgtgttact |
| 901 | aatattgatg gatatataga tacctgtatt tctgctactt ttagaagggg agcttgccat |
| 961 | gccaagaaac cccgcattac cacagccata aatgatacta gtagtgatgc tggggagtct |
| 1021 | agcggcacag gggcagaggt tgtgccattt aatgggaagg gaactaaggc tagcataaag |
| 1081 | tttcaaacta tggtaaactg gttgtgtgaa aacagagtgt ttacagagga taagtggaaa |
| 1141 | ctagttgact ttaaccagta cactttacta agcagtagtc acagtggaag ttttcaaatt |
| 1201 | caaagtgcac taaaactagc aatttataaa gcaactaatt tagtgcctac tagcacattt |
| 1261 | ttattgcata cagactttga gcaggttatg tgtattaaag acaataaaat tgttaaattg |
| 1321 | ttactttgtc aaaactatga ccccctattg gtggggcagc atgtgttaaa gtggattgat |
| 1381 | aaaaaatgtg gtaagaaaaa tacactgtgg ttttatgggc cgccaagtac aggaaaaaca |
| 1441 | aacttggcaa tggccattgc taaaagtgtt ccagtatatg gcatggttaa ctggaataat |
| 1501 | gaaaactttc catttaatga tgtagcagga aaaagcttgg tggtctggga tgaaggtatt |
| 1561 | attaagtcta caattgtaga agctgcaaaa gccattttag gcgggcaacc caccagggta |
| 1621 | gatcaaaaaa tgcgtggaag tgtagctgtg cctggagtac ctgtggttat aaccagcaat |
| 1681 | ggtgacatta cttttgttgt aagcgggaac actacaacaa ctgtacatgc taaagcctta |
| 1741 | aaagagcgca tggtaaagtt aaactttact gtaagatgca gccctgacat ggggttacta |
| 1801 | acagaggctg atgtadaaca gtggcttaca tggtgtaatg cacaaagctg ggaccactat |
| 1861 | gaaaactggg caataaacta cacttttgat ttccctggaa ttaatgcaga tgccctccac |
| 1921 | ccagacctcc aaaccacccc aattgtcaca gacaccagta tcagcagcag tggtggtgaa |
| 1981 | agctctgaag aactcagtga aagcagcttt tttaacctca tcaccccagg cgcctggaac |
| 2041 | actgaaaccc cgcgctctag tacgcccatc cccgggacca gttcaggaga atcatttgtc |
| 2101 | ggaagcccag tttcctccga agttgtagct gcatcgtggg aagaagcctt ctacacacct |
| 2161 | ttggcagacc agtttcgtga actgttagtt ggggttgatt atgtgtggga cggtgtaagg |
| 2221 | ggtttacctg tgtgttgtgt gcaacatatt aacaatagtg ggggagggtt gggactttgt |
| 2281 | ccccattgca ttaatgtagg ggcttggtat aatggatgga aatttcgaga atttacccca |
| 2341 | gatttggtgc gatgtagctg ccatgtggga gcttctaatc ccttttctgt gctaacctgc |
| 2401 | aaaaaatgtg cttacctgtc tggattgcaa agctttgtag attatgagta aaaaaagtgg |
| 2461 | caaatggtgg gaaagtgatg ataaatttgc taaagctgtg tatcagcaat ttgtggaatt |
| 2521 | ttatgaaaag gttactggaa cagacttaga gcttattcaa atattaaaag atcattataa |
| 2581 | tatttcttta gataatcccc tagaaaaccc atcctctctg tttgacttag ttgctcgtat |
| 2641 | taaaaataac cttaaaaact ctccagactt atatagtcat cattttcaaa gtcatggaca |
| 2701 | gttatctgac cacccccatg ccttatcatc cagtagcagt catgcagaac ctagaggaga |
| 2761 | aaatgcagta ttatctagtg aagacttaca caagcctggg caagttagcg tacaactacc |
| 2821 | cggtactaac tatgttgggc ctggcaatga gctacaagct gggcccccgc aaagtgctgt |
| 2881 | tgacagtgct gcaaggattc atgactttag gtatagccaa ctggctaagt tgggaataaa |
| 2941 | tccatatact cattggactg tagcagatga agagctttta aaaaatataa aaaatgaaac |
| 3001 | tgggtttcaa gcacaagtag taaaagacta ctttacttta aaaggtgcag ctgcccctgt |
| 3061 | ggcccatttt caaggaagtt tgccggaagt tcccgcttac aacgcctcag aaaaataccc |
| 3121 | aagcatgact tcagttaatt ctgcagaagc cagcactggt gcaggagggg ggggcagtaa |
| 3181 | ttctgtcaaa agcatgtgga gtgagggggc cacttttagt gctaactctg taacttgtac |
| 3241 | attttccaga cagtttttaa ttccatatga cccagagcac cattataagg tgttttctcc |
| 3301 | cgcagcgagt agctgccaca atgccagtgg aaaggaggca aaggtttgca ccatcagtcc |
| 3361 | cataatggga tactcaaccc catggagata tttagatttt aatgctttaa atttattttt |
| 3421 | ttcaccttta gagtttcagc acttaattga aaattatgga agtatagctc ctgatgcttt |
| 3481 | aactgtaacc atatcagaaa ttgctgttaa ggatgttaca gacaaaactg gagggggggt |
| 3541 | acaggttact gacagcacta cagggcgcct atgcatgtta gtagaccatg aatacaagta |
| 3601 | cccatatgtg ttagggcaag gtcaggatac tttagcccca gaacttccta tttgggtata |
| 3661 | ctttccccct caatatgctt acttaacagt aggagatgtt aacacacaag gaatttctgg |
| 3721 | agacagcaaa aaattagcaa gtgaagaatc agcattttat gttttggaac acagttcttt |
| 3781 | tcagctttta ggtacaggag gtacagcatc tatgtcttat aagtttcctc cagtgccccc |
| 3841 | agaaaattta gagggctgca gtcaacactt ttatgaaatg tacaatccct tatacggatc |
| 3901 | ccgcttaggg gttcctgaca cattaggagg tgacccaaaa tttagatctt taacacatga |
| 3961 | agaccatgca attcagcccc aaaacttcat gccagggcca ctagtaaact cagtgtctac |
| 4021 | aaaggaggga gacagctcta atactggagc tggaaaagcc ttaacaggcc ttagcacagg |
| 4081 | tacctctcaa aacactagaa tatccttacg ccctgggcca gtgtctcagc cataccacca |
| 4141 | ctgggacaca gataaatatg tcacaggaat aaatgccatt tctcatggtc agaccactta |
| 4201 | tggtaacgct gaagacaaag agtatcagca aggagtgggt agatttccaa atgaaaaaga |
| 4261 | acagctaaaa cagttacagg gtttaaacat gcacacctac tttcccaata aaggaaccca |
| 4321 | gcaatataca gatcaaattg agcgccccct aatggtgggt tctgtatgga acagaagagc |
| 4381 | ccttcactat gaaagccagc tgtggagtaa aattccaaat ttagatgaca gttttaaaac |
| 4441 | tcagtttgca gccttaggag gatggggttt gcatcagcca cctcctcaaa tatttttaaa |
| 4501 | aatattacca caaagtgggc caattggagg tattaaatca atgggaatta ctaccttagt |
| 4561 | tcagtatgcc gtgggaatta tgacagtaac tatgacattt aaattggggc cccgtaaagc |
| 4621 | tacgggacgg tggaatcctc aacctggagt atatcccccg cacgcagcag gtcatttacc |
| 4681 | atatgtacta tatgacccca cagctacaga tgcaaaacaa caccacagac atggatatga |
| 4741 | aaagcctgaa gaattgtgga cagccaaaag ccgtgtgcac ccattgtaaa cactccccac |
| 4801 | cgtgccctca gccaggatgc gtaactaaac gcccaccagt accacccaga ctgtacctgc |
| 4861 | cccctcctgt acctataaga cagcctaaca caaaagatat agacaatgta gaatttaagt |
| 4921 | acttaaccag atatgaacaa catgttatta gaatgttaag attgtgtaat atgtatcaaa |
| 4981 | atttagaaaa ataaacattt gttgtggtta aaaaattatg ttgttgcgct ttaaaaattt |
| 5041 | aaaagaagac accaaatcag atgccgccgg tcggccggta ggcgggactt ccggtacaag |
| 5101 | atggcggaat tc |

Alignment for purposes of determining percent nucleic acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for measuring alignment, including any algorithms needed to achieve maximal alignment over the full-length of the sequences being compared.

For purposes herein, the % nucleic acid sequence identity of a given nucleic acid sequence A to, with, or against a given nucleic acid sequence B (which can alternatively be phrased as a given nucleic acid sequence A that has or comprises a certain % nucleic acid sequence identity to, with, or against a given nucleic acid sequence B) is calculated as follows:

100 times the fraction W/Z

where W is the number of nucleotides scored as identical matches by the sequence alignment program in that program's alignment of A and B, and where Z is the total number of nucleotides in B. It will be appreciated that where the length of nucleic acid sequence A is not equal to the length of nucleic acid sequence B, the % nucleic acid sequence identity of A to B will not equal the % nucleic acid sequence identity of B to A.

The term “primary cell” as used herein refers to a cell obtained from a primary source such as a tissue or blood sample from an organism, preferably an animal. In an embodiment, the animal is a human.

“Recombinant” refers to a polynucleotide or polypeptide encoded by a polypeptide that has been isolated and/or altered by the hand of man or a B19 clone encoded by such a polynucleotide. A DNA sequence encoding all or a portion of a B19 viral genome may be isolated and combined with other control sequences in a vector. The other control sequences may be those that are found in the naturally occurring gene or others. The vector provides for introduction into host cells and amplification of the polynucleotide. The vectors described herein for B19 clones are introduced into cells and cultured under suitable conditions as known to those of skill in the art. Preferably, the host cell is a bacterial cell or a permissive cell.

The term “transformation” as used herein refers to introducing exogenous DNA into a bacterial cell so that the DNA is replicable or into a eukaryotic cell, either as an extrachromosomal element or by chromosomal integration. The introduced DNA is transcribed and expressed by the cell. Depending on the host cell used, transformation is done using standard techniques appropriate to such cells. Methods for transformation include, but are not limited to, electroporation, viral vectors, liposomal vectors, gene gun, microinjection and transforming viruses.

The term “transfection” as used herein refers to introducing exogenous DNA into a eukaryotic cell so that the DNA is replicable, either as an extrachromosomal element or by chromosomal integration. Depending on the host cell used, transfection is done using standard techniques appropriate to such cells. Methods for transfecting eukaryotic cells include polyethyleneglycol/DMSO, liposomes, electroporation, and electrical nuclear transport.

The term “transfection efficiency” as used herein means the percentage of total cells contacted with a nucleic acid, such as a plasmid, that take up one or more copies of the plasmid. Transfection efficiency can also be expressed as the total number of cells that take up one or more copies of the plasmid per μg of plasmid. If the plasmid contains a reporter gene, transfection efficiency of cells can also be expressed in units of expression of the reporter gene per cell.

The term “replicable vector,” as used herein, is intended to refer to a nucleic acid molecule capable of transporting another nucleic acid to which it has been linked into a cell and providing for amplification of the nucleic acid. One type of vector is a “plasmid”, which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. In the present specification, “plasmid” and “vector” may be used interchangeably as the plasmid is the most commonly used form of vector. In some embodiments, the vector is a vector that can replicate to high copy number in a cell.

The term “viral vector” or “recombinant viral vector” as used herein refer to a virus that has been genetically altered such that a nucleic acid sequence has been integrated to the viral genome whereby the virus serves as a vector to introduce the integrated nucleic acid sequence into a host cell. Examples of viral vectors are adenoviral vectors, adeno-associated viral vectors (AAV), lentiviral vectors and retroviral vectors.

“ITR” or “ITR sequence” refers to an inverted terminal repeat of nucleotides in a nucleic acid such as a viral genome. The ITRs include an imperfect palindrome that allows for the formation of a double stranded hairpin with some areas of mismatch that form bubbles. The ITRs serve as a primer for viral replication and contain a recognition site for NS protein that may be required for viral replication and assembling. In some embodiments, the location and number of the bubbles or areas of mismatch are conserved as well as the NS binding site. The NS binding site provides for cleavage and replication of the viral genome.

### II. Methods and Cells Permissive for B19 Virus Infection

Parvovirus B19 (B19) may infect permissive cells but the amount of infectious virus produced in these cells may be very small. Cells and methods for consistently producing B19 in useful quantities in cell culture are limited. Utilizing the methods of the disclosure, cells that produce useful quantities of B19 were, isolated and in some embodiments, immortalized.

B19 produced by the cells and methods of the invention can be utilized in a variety of assays and to develop therapeutic products. An in vitro system for producing infectious virus particles can be used in screening methods to diagnose disease and/or to identify agents, such as antibodies or antisense molecules that can inhibit viral infectivity or reproduction. Infectious virus produced by the cells and methods of the invention and/or infectious virus in a host cell of the invention can be utilized to form immunogenic compositions to prepare therapeutic antibodies or vaccine components. The ability to produce significant amounts of infectious virus in vitro is also useful to develop attenuated strains of the virus that may be utilized in vaccines.

Biomarkers of B19 infected cells can also be useful to identify parvovirus infected cells. Methods of detecting expression or activity of differentially expressed genes in virus infected cells are provided herein.

A. Methods for Producing Parvovirus B19 in CD36+ Erythroid Progenitor Cells.

The disclosure provides methods for producing parvovirus B19 in CD36⁺ erythroid progenitor cells. In some embodiments, the CD36⁺ cells are also CD34⁻ and/or CD133⁻. In some embodiments, a method is directed to producing B19 viral genomes, virus particles, viral transcripts, and/or clones. The methods of the disclosure comprise introducing parvovirus B19 genomes into erythroid progenitor cells. In an embodiment, the CD36⁺ erythroid progenitor cells are non-enucleated, globoside positive, and optionally, comprise hemoglobin in a subset of the cell population. In some embodiments, the erythroid progenitor cell population has about the same percentage of cells that are CD36⁺ and globoside. In some embodiments, the population has at least 25 to 60% of the cells positive for globoside and CD36. In some embodiments, the population has at least 60% of the cells positive for globoside and CD36 and at least 50% cells positive for glycophorin (CD235a). In some embodiments, at least 25% to 100% of the erythroid progenitor cells are CD36+ and globoside+cells, and less than 70% of the cell population are CD33⁺.

In an embodiment, the CD36⁺ erythroid progenitor cells are CD34⁻, CD44⁺, CD235a⁺, CD19⁻, and CD3⁻. In an embodiment, the CD36⁺ erythroid progenitor cells are CD36⁺, CD44⁺, CD235a⁺, CD34⁻, CD19⁻, CD10⁻, CD4⁻, CD3⁻, and CD2.

### Cells

The erythroid progenitor cells can be produced from hematopoietic stem cells. The hematopoietic stem cells can be pluripotent or lineage restricted. In some embodiments, the hematopoietic stem cells are isolated from bone marrow, peripheral blood, embryonic tissue, fetal tissue, or umbilical cord blood. Methods for isolating stem cells are known and include, for example, magnetic cell sorting, microbead selection, and ficoll density gradient separation. In an embodiment, the stem cells are CD34⁺ hematopoietic stem cells. In an embodiment, the stem cells are CD133⁺ hematopoietic stem cells. In an embodiment, the stem cells are CD133⁺ and CD34⁺ hematopoietic stem cells. Other marker as described herein may be also be used to select or characterize the hematopoietic stem cells.

When a population of cells is enriched for CD34+, CD133+, or both only a subset of the cells are hematopoietic stem cells. The CD34⁺, CD133+, or cells with both are a mixture of hematopoietic stem cells and cells that are in the process of differentiating, which includes myeloid lineage and lymphoid lineage pluripotent stem cells and myeloid lineage restricted and lymphoid lineage restricted stem cells. Enriching for CD34+, CD133+, or both positive cells results in a mixture of hematopoietic stem cells and precursor cells such as pluripotent stem cells, lymphoid precursor cells and various myeloid lineage restricted stem cells that can differentiate into CD36⁺ erythroid progenitor cells. Cells selected for CD34+ or CD133+ enrich for the same subpopulation of hematopoietic stem cells.

Kits for isolating CD34⁺ or CD133⁺ cells are commercially available, for example, from Miltenyi Biotech (Auburn, Calif.). In an embodiment, CD34⁺ stem cells are isolated by magnetic microbead selection (Giarrantana et al., 2005, Nature Biotech., 23:69-74; Freyssinier et al., 1999, Brit. J. Haemotol., 106:912-922). In an embodiment, the pluripotent stem cells are myeloid precursor cells (CFU-S). In an embodiment, the lineage restricted stem cells are BFU-E or CFU-E. In an embodiment, CD133⁺ stem cells are isolated by magnetic microbead selection using kits for isolating CD133⁺ cells commercially available, for example, from Miltenyi Biotech (Auburn, Calif.).

Methods for generating and amplifying a population of human erythroid progenitor cells from hematopoietic stem cells are known. See, for example, Giarrantana et al., 2005, Nature Biotech., 23:69-74 and Freyssinier et al., 1999, Brit. J. Haemotol., 106:912-922. In an embodiment, the CD36⁺ erythroid progenitor cells are produced from CD34⁺ hematopoietic stem cells isolated from G-CSF mobilized peripheral blood stem cells. The CD34⁺ hematopoietic stem cells can be frozen cells that have been thawed or freshly isolated cells. In an embodiment, the CD36⁺ erythroid progenitor cells are produced from CD133⁺ hematopoietic stem cells isolated from G-CSF mobilized peripheral blood stem cells.

In an embodiment, the hematopoietic stem cells are cultured at an initial density of about 10⁴ cells/mL to about 1 to 100×10⁵ cells in expansion media under conditions that allow for expansion and differentiation of the cells, diluted 1:5 in expansion media and the diluted cells are cultured in expansion media under conditions that allow for expansion and differentiation. In an embodiment, the hematopoeitic stem cells are cultured at an initial density of about 10⁴ cells/mL to about 1 to 100×10⁵ cells in expansion media under conditions that allow for expansion and differentiation of the cells and can be frozen and thawed for further expansion and differentiation. In an embodiment, the hematopoietic stem cells are cultured at an initial density of about 10⁴ cells/mL and allowed to grow for at least 4 to 20 days in expansion media under conditions that allow for expansion and differentiation of the cells and can be frozen and thawed for further expansion and differentiation.

CD36 is used as a marker for erythroid progenitor cells. CD19, CD3, and CD2 are cell surface markers for lymphocytes, and erythroid progenitor cells do not have these markers and as such can be used to distinguish these cells from lymphoid lineage cells. CD44 is a cell surface marker for leukocytes and erythrocytes. CD235a (glyophorin A) is found on erythroid progenitor cells. CD71 is a marker for the transferrin receptor. In an embodiment, the CD36⁺ erythroid progenitor cells are globoside+, CD36⁺, CD34⁻, CD19⁻, and CD3⁻. In an embodiment, the CD36⁺ erythroid progenitor cells are globoside+, CD36⁺, CD34⁻, CD19⁻, and CD3⁻. In an embodiment, the CD36⁺ erythroid progenitor cells are CD36⁺, CD44⁺, CD235a⁺, CD34⁻, CD19⁻, CD10⁻, CD4⁻, CD3⁻, and CDT.

In an embodiment, the CD36⁺ erythroid progenitor cells are non-enucleated, globoside positive, and optionally comprise hemoglobin. In an embodiment, the population of CD36⁺ erythroid progenitor cells comprises less than 70% CD33⁺ cells, and more preferably 80, 70, 60, 50, or 40% or any number % less than 70 of CD33⁺ cells. In some embodiments, at least 25% to 100% of the erythroid progenitor cells are CD36+ and globoside+cells, and less than 70% of the cell population are CD33+. In an embodiment, the population of CD36⁺ erythroid progenitor cells comprises 30% CD71⁺ cells, and more preferably 40, 50, 60, 70, 80, 90 or more or any number of % greater than 30% up to 100% of CD71⁺ cells. In some embodiments, the erythroid progenitor cell population has about the same percentage of cells that are CD36+ and globoside+. In some embodiments, the population has at least 25 to 100% of the cells positive for globoside and CD36. In some embodiments, the population has at least 60% of the cells positive for globoside and CD36 and at least 50% cells positive for glycophorin by day 8 in culture.

In another embodiment, the hemapoeitic stem cells are cultured for about at least 4 to 26 days in expansion media under conditions that allow for expansion and differentiation of the cells. The cells are cultured at a low concentration (˜10⁴ cells/mL) and then the culture volume is expanded in expansion media which allows for continued expansion and differentiation. In some embodiments, the cells are cultured for about 2-4 days, the culture volume expanded at least 2-5 fold in expansion medium for an additional 2-18 days.

In some embodiments, the culture comprises at least about 25 to 100% CD36⁺ cells, more preferably about 60%, 70%, 80%, 90%, 95%, 98% or 100% of CD36⁺ cells. The % of CD36⁺ cells can include any number from 25 to 100% of the cells are CD36⁺. The proportion of CD36⁺ cells in the population can be determined using standard methodologies, such as FACS analysis.

In some embodiments, the expansion media comprises stem cell factor (SCF), interleukin 3 (IL-3), and/or erythropoietin. The amounts of the growth factors and media components can be varied in accord with what is known in the art for culturing hematopoietic stem cells. In some embodiments, the expansion media comprises stem cell factor (SCF), interleukin 3 (IL-3), hydrocortisone, and/or erythropoietin. In some embodiments, the expansion media comprises bovine serum albumin (BSA), insulin, transferrin, ferrous sulfate, ferric nitrate, insulin, hydrocortisone, stein cell factor (SCF), interleukin 3 (IL-3), and/or erythropoietin. In an embodiment, the expansion media comprises about 10 mg/ml BSA, about 10 μg/ml recombinant human insulin, about 200 μg/ml human transferrin, about 900 ng/ml ferrous sulfate, about 90 ng/ml ferric nitrate, about 10⁻⁶ M hydrocortisone, about 5 ng/ml IL-3, about 100 ng/ml recombinant human SCF, and about 3 IU/ml recombinant human erythropoietin. In another embodiment, the expansion medium comprises BIT 9500 media (StemCell Tech. Inc., Vancouver, British Columbia) diluted 1:5 in AMEM (Mediatech Inc., Herndon, Va.) and supplemented with 10⁻⁶ M hydrocortisone, 5 ng/ml human IL-3, 100 ng/ml recombinant human stem cell factor, 3 IU/ml recombinant human erythropoietin, 900 ng/ml ferrous sulfate, and 90 ng/ml ferric nitrate and has a final concentration of 10 mg/ml deionized BSA, 10 μg/ml recombinant human insulin, and 200 μg/ml iron saturated human transferrin. Ranges of the concentration of the components in the expansion media can be varied as is known to those of skill in the art.

When the cells become CD36⁺, the cells are permissive for B19 virus replication. In an embodiment, at least some of the cells are actively dividing when infected. In some embodiments, B19 virus can be introduced into CD36⁺ cells after 1 day in culture to about 8 days after the cell culture has reached about 25% or greater CD36+ cells. In an embodiment, the cells are infected from day 8 to day 20 in culture. The cells may be transformed and/or immortalized as described herein, and then the B19 virus can be introduced at a later time point or can be cultured for a longer period of time post transformation.

B. Permissive Cells

The disclosure also provides erythroid progenitor cells that are permissive for B19 infection. The CD36⁺ erythroid progenitor cells of the invention can be produced from cells as described herein. In some embodiments, the CD36⁺ erythroid progenitor cells are CD36⁺ and CD34⁻. CD19, CD3, and CD2 are cell surface markers for lymphocytes cells and can be used to distinguish erythroid progenitor cells from lymphoid lineage cells. CD44 is a cell surface marker for leukocytes and erythrocytes. CD235a is a cell surface marker for glyophorin A typically found on erythroid cells. In some embodiments, the erythroid progenitor cell population has about the same percentage of cells that are CD36+ and globoside+. In some embodiments, the population has at least 25 to 60% of the cells positive for globoside and CD36. In some embodiments, at least 25% to 100% of the erythroid progenitor cells are CD36+ and globoside+cells, and less than 70% of the cell population are CD33+. In some embodiments, the population has at least 60% of the cells positive for globoside and CD36 and at least 50% cells positive for glycophorin.

In an embodiment, the CD36⁺ erythroid progenitor cells are CD36, CD34⁻, CD19⁻, and CD3⁻. In an embodiment, the CD36⁺ erythroid progenitor cells are CD36⁺, CD34⁻, CD133⁻, CD19⁻ and CD3′. In an embodiment, the CD36⁺ erythroid progenitor cells are CD36⁺, CD44⁺, CD235a⁺, CD34⁻, CD19⁻, CD10⁻, CD4⁻, CD3⁻, and CDT. In an embodiment, the CD36⁺ cells comprise hemoglobin and/or globoside.

In an embodiment, the CD36⁺ erythroid progenitor cells are BFU-E, CFU-E, proerythroblasts, or erythroblasts. In an embodiment, the CD36⁺ erythroid progenitor cells are non-enucleated and comprise hemoglobin and/or globoside. The CD36⁺ erythroid progenitor cells can be infected with B19 as described herein.

Replication of B19 in reported permissive cell lines is known to be limited. Examples of reported permissive cell lines include, but are not limited to, megakaryoblastoid cell lines such as UT7/Epo, UT7/Epo-S1, and MB-O2 and erythroleukemic cell lines such as KU812Ep6 and JK-1. Previous studies have indicated that UT7/Epo-S1 cells are the most permissive cells for B19 infection (Wong, et. al., 2006, Journal of Clinical Virology, 35:407-413). In some embodiments, replication of the B19 genome in the erythroid progenitor cells of the disclosure is greater than replication of the viral genome in UT7/Epo-S1 cells. In an embodiment, replication of B19 genome in the erythroid progenitor cells is at least 10 fold greater, at least 50 fold greater, at least 100 fold greater, at least 200 fold greater, at least 300 fold greater, at least 400 fold greater, or at least 500 fold greater than the replication of B19 genome in UT7/Epo-S1 cells. In some embodiments, production of B19 genome in the CD36⁺ erythroid progenitor cells of the invention is greater than production of B19 genome in UT7/Epo-S1 cells. In an embodiment, production of B19 genome in the CD36⁺ erythroid progenitor cells of the invention is at least 0.5 log, at least 1.0 log, at least 1.5 log, at least 2.0 log, or at least 2.5 log greater that the production of B19 genome in UT7/Epo-S1 cells.

In an embodiment, the CD36⁺ erythroid progenitor cells of the disclosure are secondary cells or immortalized cells. Methods for immortalizing cells in culture are known. See, for example, Culture of Immortalized Cells, Freshney and Freshney Eds., Wiley Publishing Inc, Indianapolis, Ind., 1996 and Hahn, W C, 2002, Mol. Cells, 13:351-361. Methods for immortalizing cells include, but are not limited to, transforming cells with a vector comprising a polynucleotide that inactivates tumor suppressor genes in the transformed cells that results in a replicative senescent state or a polynucleotide that regulates the expression or activity of telomerase. Examples of polynucleotides that inactivate tumor suppressor genes include, but are not limited to, simian virus (SV40) T antigen gene, adenovirus E1A or E1B gene, and human papillomavirus type 16 (HPV-16) E6 or E7 gene. One example of a polynucleotide that regulates expression or activity of telomerase is telomerase reverse transcriptase (TERT). TERT is commercially available, for example, from Geron Corp., Menlo Park, Calif. In other embodiments, Epstein Barr virus is used to immortalize the cells.

In an embodiment, the vector is a recombinant plasmid, a recombinant virus, or a retrovirus. In an embodiment, the viral vector is an adenoviral vector, lentiviral vector, AAV vector, Epstein Barr Virus, or retroviral vector. A eukaryotic expression plasmid containing human TERT cDNA is commercially available from American Type Culture Collection (Manassas, Va.: catalog number ATCC® MBA-141). Other viral vectors are commercially available.

In some embodiments, an erythroid progenitor cell is a secondary cell. In an embodiment, secondary cells are generated by transforming primary cells with a vector comprising a polynucleotide that inactivates tumor suppressor genes in the transformed cells that results in a replicative senescent state or a polynucleotide that regulates the expression or activity of telomerase or is Epstein Barr virus.

In another embodiment, secondary erythroid progenitor cells can also be generated by culturing primary cells under conditions that result in increased number of cell divisions or life span. In an embodiment, a secondary cell can divide at least 2 to about 100 times, more preferably about 2 to 50 times, more preferably 2 to 15. In an embodiment, a secondary cell can divide indefinitely. In an embodiment, the doubling time of the secondary cells is about 12 hours, about 16 hours, about 24 hours, about 30 hours, or about 36 hours.

The secondary erythroid progenitor cells are cultured in an appropriate growth medium that provides for increased number of generations. In some embodiments, the expansion media comprises stem cell factor (SCF), interleukin 3 (IL-3), and/or erythropoietin. The amounts of the growth factors may be varied as is known in the art. In some embodiments, the expansion media comprises stem cell factor (SCF), interleukin 3 (IL-3), hydrocortisone, and/or erythropoietin. In some embodiments, the expansion media comprises bovine serum albumin (BSA), insulin, transferrin, ferrous sulfate, ferric nitrate, insulin, hydrocortisone, stem cell factor (SCF), interleukin 3 (IL-3), and/or erythropoietin. In an embodiment, the expansion media comprises about 10 mg/ml BSA, about 10 μg/ml recombinant human insulin, about 200 μg/ml human transferrin, about 900 ng/ml ferrous sulfate, about 90 ng/ml ferric nitrate, about 10⁻⁶ M hydrocortisone, about 5 ng/ml (IL-3), about 100 ng/ml recombinant human SCF, and about 3 IU/ml recombinant human erythropoietin. In another embodiment, the expansion medium comprises BIT 9500 media (StemCell Tech. Inc., Vancouver, British Columbia) diluted 1:5 in AMEM (Mediatech Inc., Herndon, Va.) and supplemented with 10⁻⁶ M hydrocortisone, 5 ng/mL human IL-3, 100 ng/ml recombinant human stem cell factor, 3 IU/ml recombinant human erythropoietin, 900 ng/ml ferrous sulfate, and 90 ng/ml ferric nitrate and has a final concentration of 10 mg/ml deionized BSA, 10 μg/ml recombinant human insulin, and 200 μg/ml iron saturated human transferrin.

In some embodiments, the secondary erythroid progenitor cells can be cultured from about 1 to 15 days. In an embodiment, the secondary CD36⁺ erythroid progenitor cells of the invention have a life span of about 10 to about 30 days. In an embodiment, the secondary CD36⁺ erythroid progenitor cells of the invention have a life span of about 30 days to about 40 days, of about 40 days to about 50 days, of about 50 days to about 60 days, of about 60 days to 70 days, of about 70 days to about 80 days, of about 80 days to about 90 days, or of about 90 days to about 100 days. In an embodiment, the secondary CD36⁺ erythroid progenitor cells of the invention have a life span of at least 30 days, of at least 40 days, of at least 50 days, of at least 60 days, of at least 70 days, of at least 80 days, of at least 90 days, of at least 100 days, of at least 150 days, of at least 200 days, of at least 250 days, of at least 300 days, or of at least 350 days.

In an embodiment, the CD36⁺ erythroid progenitor cells of the invention can undergo at least 10 doublings, at least 20 doublings, at least 30 doubling, at least 40 doublings, at least 50 doublings, at least 60 doublings, at least 70 doublings, at least 80 doublings, at least 90 doublings, at least 100 doublings, at least 200 doublings, at least 300 doublings, at least 400 doublings, at least 500 doublings, at least 600 doublings, at least 700 doublings, at least 800 doublings, at least 900 doublings, at least 1000 doublings, at least 1500 doublings, at least 2000 doublings, at least 2500 doublings, at least 3000 doublings, at least 4000 doublings, at least 5000 doublings, or at least 10,000 doublings.

In an embodiment, the CD36⁺ erythroid progenitor cells of the invention are immortalized with a viral vector comprising a polynucleotide encoding SV40 large T-antigen. Viral vectors encoding SV40 large T-antigen are known. See, for example, Gluzman et al., 1980, Proc. Nall. Acad. Sci. U.S.A., 77:3898-3902. While not wishing to be bound by theory, it is believed that the SV40 large T antigen transforms the cells into tumor-like cells, which like cancer cells, grow rapidly and allow the cells to continue multiplying for an extended period of time. In an embodiment, the viral vector is an adenovirus, lentivirus, adeno-associated virus (AAV), or retrovirus. In an embodiment, the CD36⁺ erythroid progenitor cells of the invention are contacted with a viral vector when the population has at least 25% CD36+ cells. In some embodiments, at least 25% to 100% of the erythroid progenitor cells are CD36+ and globoside+cells, and less than 70% of the cell population are CD33+. In an embodiment, the CD36⁺ erythroid progenitor cells of the invention are contacted with a viral vector after 8 days in expansion media. The expansion media comprises cytokines and growth factors that induce the hemapoeitic stem cells to differentiate into the erythroid progenitor cells of the disclosure. In an embodiment, the expansion media comprises SCF, IL-3, and/or erythropoietin and/or hydrocortisone.

In an embodiment, immortalization of the CD36⁺ erythroid progenitor cells as described herein inhibits further differentiation of the cells. In an embodiment, immortalization of the erythroid progenitor cells as described herein maintains the cells as CD36⁺ erythroid progenitor cells and inhibits differentiation of the cells into erythrocytes. In a specific embodiment, the cells may be frozen after about 1 to about 6 passages and the frozen cells may be thawed and cultured. In an embodiment, immortalization of the erythroid progenitor cells maintains the cells as CD36⁺ erythroid progenitor cells and inhibits differentiation of the cells into erythrocytes even after one or more passages or plating the cells from frozen stocks subjected to one or more freeze/thaw cycles. In an embodiment, the immortalized CD36⁺ erythroid progenitor cells of the invention are BFU-E, CFU-E, proerythroblasts, or erythroblasts. In an embodiment, the immortalized CD36⁺ erythroid progenitor cells are BFU-E, CFU-E, proerythroblasts, or erythroblasts erythroid progenitors even after one or more passages or plating the cells from frozen stocks subjected to one or more freeze/thaw cycles.

The secondary or immortalized CD36⁺ erythroid progenitor cells of the disclosure maintain permissiveness for B19 infection. In an embodiment, the secondary or immortalized CD36⁺ erythroid progenitor cells of the invention maintain genetic stability and permissiveness for B19 infection after multiple passages. B19 virus can be introduced into the cells at any time, such as when the cells have reached about 25 to 100% CD36⁺ cells, more preferably about 70 to 100%, or even 90 to 100% CD36⁺. In an embodiment, the cells can be infected up to 13 days, up to 15 days, up to 20 days, up to 25 days, or up to 30 days. In an embodiment, the cells remain permissive for infection indefinitely.

In an embodiment, replication of B19 genome in the secondary or immortalized CD36⁺ erythroid progenitor cells of the invention is at least 100 to 1000 fold greater than the replication of B19 genome in UT7/Epo-S1 cells depending on the concentration of input virus. In some embodiments, production of B19 in the secondary or immortalized CD36⁺ erythroid progenitor cells of the invention is greater than production of B19 in UT7/Epo-S1 cells. In an embodiment, production of B19 in the secondary or immortalized CD36⁺ erythroid progenitor cells of the invention is at least 2 log to 3 logs greater that the production of B19 in UT7/Epo-S1 cells depending on the concentration of input virus.

C. B19 Virus

The erythroid progenitor cells as described herein can be infected by contacting the cells with B19 or introducing a vector comprising an infectious clone of B19 into the cells. B19 can be naturally occurring or a variant thereof. B19 viral DNA can be isolated from infected humans or cells as described, for example, in Wong, et. al., 2006, Journal of Clinical Virology, 35:407-413 or can be prepared as described, for example, in U.S. 20060008469 or Zhi et al., 2004, Virology, 318:142-152. Utilizing an infectious clone allows introduction of the viral genome into a cell without the need for entry mediated by viral proteins such as the capsid protein and/or the presence of globoside on the cell.

In some embodiments, the reference sequence may be human parvovirus B19-Au (GeneBank accession number M13178; SEQ ID NO:307), which lacks intact 1TRs at both 5′ and 3′ ends of the genome and the naturally occurring variants have at least 90% sequence identity to the reference sequence. In other cases, a variant may be prepared by altering or modifying the nucleic acid sequence of the viral genome including by addition, substitution, and deletion of nucleotides. In that case, the reference sequence can be that of parvovirus B19 comprising a polynucleotide sequence of SEQ ID NO:307. In some embodiments, a parvovirus genome has at least 90% sequence identity, more preferably at least 95%, or greater sequence identity to that of a parvovirus B19 genome comprising a nucleic acid sequence of a B19 comprising a polynucleotide sequence of SEQ ID NO:307 or SEQ ID NO:308.

In an embodiment, a vector identified as pB19-M20 comprises a full length B19 having a SEQ ID NO:307 but with a change at nucleotide 2285 from a cytosine to a thymine, resulting in conversion of a BsrI site to a Dde site (U.S. 20060008469; Zhi et al., 2004, Virology, 318:142-152).

An infectious clone of B19 can be a full-length genome or portion of a genome of a parvovirus B19 isolate cloned into a replicable vector that provides for amplification of the viral genome in a cell. Infectious B19 clones and methods of making infectious B19 clones are described, for example, in U.S. 20060008469, Zhi et al., 2004, Virology, 318:142-152, and Zhi, et. al., 2006, Journal of Virology, in press. In some embodiments, a portion of the B19 genome comprises or consists of nucleic acid sequence encoding at least one P6 promoter ITR, VP2, VP1, NS, and 11-kDa in a single replicable vector. In some embodiments, the replicable vector includes at least one of an origin of replication, a selective marker gene, a reporter gene, a P6 promoter or the ITRs.

In other embodiments, the viral genome is a full-length genome. A full length genome comprises a complete coding sequence of a viral genome that comprises at least 75% or greater of the nucleotide sequence that forms the hairpin of the ITR at the 5′ end and 3′ end of the genome. In an embodiment, the coding sequence comprises nucleic acid sequence encoding VP1, VP2, NS, 11-10a protein, 7.5-kDa protein, and putative protein X.

In an embodiment, the parvovirus B19 genome comprises one or more ITR sequences. Preferably, the B19 genome comprises an ITR sequence at the 5′ end and the 3′ end. An ITR may be about 350 nucleotides to about 400 nucleotides in length. An imperfect palindrome may be formed by about 350 to about 370 of the distal nucleotides, more preferably about 360 to about 365 of the distal nucleotides. Preferably the imperfect palindrome forms a double-stranded hairpin. In an embodiment, the ITRs are about 383 nucleotides in length, of which about 365 of the distal nucleotides are imperfect palindromes that form double-stranded hairpins. In another embodiment, the ITRs are about 381 nucleotides in length, of which about 361 of the distal nucleotides are imperfect palindromes that form double-stranded hairpins. In some embodiments, a B19 genome comprises at least 75% of the nucleotide sequence that forms the hairpin in the ITR at the 5′ end and 3′ end of the genome. In other embodiments, the ITRs may have 1 to about 5 nucleotides deleted from each end. The lilts may be in the “flip” or “flop” configuration.

The B19 clones may be synthesized or prepared by techniques well known in the art. Some nucleotide sequences for parvovirus B19 genomes are known and readily available, for example, on the Internet at GenBank (accessible at www-ncbi-nlm-nihgov/entrez). The nucleotide sequences encoding the B19 clones of the invention may be synthesized or amplified using methods known to those of ordinary skill in the art including utilizing DNA polymerases in a cell free environment. Methods for preparing, amplifying, and producing vectors comprising a B19 genome are disclosed, for example, in U.S. 20060008469 and Zhi et al., 2004, Virology, 318:142-152.

The B19 clones can be produced from a virus obtained from biological samples. The B19 virus isolates can be obtained from biological samples obtained from infected humans. The biological sample can include blood, serum, tissue, biopsy, urine, and the like.

The polynucleotides may be produced by standard recombinant methods known in the art, such as polymerase chain reaction (Sambrook, et al., 1989, Molecular Cloning, A Laboratory Manual, Vols. 1-3, Cold Spring Harbor Press, Cold Spring Harbor, N.Y. Methods of altering or modifying nucleic acid sequences are also known to those of skill in the art.

In some embodiments, the parvovirus B19 genome is introduced into the cell by uptake into the cell through a receptor, such as globoside. In some cases, the cells are contacted with a biological sample comprising infectious parvovirus B19 virus. In an embodiment, cells are contacted with about 100 or more genomes/ml of infectious virus, more preferably about more preferably 10³ to 10⁶ genomes/ml. In an embodiment, cells are contacted with an MOI of 0.01 to 100,000.

D. Introduction of B19 Virus into Cells and Methods of Detection

A method of the disclosure comprises introducing a vector comprising an infectious clone of parvovirus B19 or all or a portion of a viral genome into erythroid progenitor cells or infecting erythroid progenitor cells with parvovirus B19 particles, culturing the cells under conditions that provide for replication of the viral genome, and optionally, detecting production of viral genome or particles. In an embodiment, the method comprises introducing a vector comprising all or a portion of a viral genome into CD36⁺ erythroid progenitor cells; incubating the cells for a sufficient time to produce infectious virus; and detecting production of infectious virus. The CD36+ cells can be primary cells or cells transformed with the vectors as described herein. In some embodiments the CD36+ cells (whether primary, secondary or immortalized) have been cultured for at least 7 days and up to 40 days.

Introduction of B19 genome or a vector comprising a B19 genome into a eukaryotic host cell can be facilitated by calcium phosphate transfection, DEAE-dextran mediated transfection, cationic lipid-mediated transfection, electroporation, electrical nuclear transport, chemical transduction, electrotransduction, infection, or other methods. Such methods are described in standard laboratory manuals such as Sambrook, et al., 1989, Molecular Cloning, A Laboratory Manual, Vols. 1-3, Cold Spring Harbor Press, Cold Spring Harbor, N.Y. or Davis et al., 1986, Basic Methods in Molecular Biology. In an embodiment, the host cell is a CD36⁺ erythroid progenitor cell.

Commercial transfection reagents, such as Lipofectamine (Invitrogen, Carlsbad, Calif.) and FuGENE 6™ (Roche Diagnostics, Indianapolis, Ind.), are also available. Preferably transfection efficiency of the host cells is about 15% or greater, more preferably about 20% or greater, more preferably about 30% or greater, more preferably about 40% or greater, more preferably about 50% or greater, more preferably about 70% or greater.

In some embodiments, a high efficiency of introduction of the vector into the CD36⁺ erythroid progenitor cells is desired. Preferably, the method of introduction employed achieves a transfection efficiency of at least about 15% to 100% efficiency, more preferably about 30 to 50% efficiency. The method is also selected to minimize cytotoxicity to the cells. Preferably, about 20% or greater of the cells are viable and more preferably about 50% of the cells or greater. In some embodiments, the vector may be cut with one or more restriction enzymes to enhance viral replication.

In an embodiment, CD36⁺ erythroid progenitor cells are transfected with an electric current. Methods of transfecting eukaryotic cells utilizing an electric current are known in the art, such as for example, electroporation (Sambrook, et al., 1989, Molecular Cloning, A Laboratory Manual, Vols. 1-3, Cold Spring Harbor Press, Cold Spring Harbor, N.Y. or Davis et al., 1986, Basic Methods in Molecular Biology) and electrical nuclear transport (U.S. 20040014220).

In an embodiment, the CD36⁺ erythroid progenitor cells are transfected by electrical nuclear transport. The cells are exposed to an electrical pulse comprising a field strength of about 2 kV/cm to about 10 kV/cm, a duration of about 10 μsec to about 200 μsec, and a current of at about 1 A to about 2.5 A followed by a current flow of about 1 A to about 2.5 A for about 1 msec to about 50 msec. A buffer suitable for use in electrical nuclear transport comprises 0.42 mM Ca(NO₃)₂, 5.36 mM KCl, 0.41 mM MgSO₄, 103 mM NaCl, 23.8 mM NaHCO₃, 5.64 mM Na₂HPO₄, 11.1 mM d(+) glucose, 3.25 μM glutathione, 20 mM Hepes, and pH 7.3. Following transfection, the permissive cells may be incubated for about 10 min at 37° C. before being plated in prewarmed (37° C.) culture medium with serum and incubated at 37° C.

Commercially available devices and buffer systems for electrical nuclear transport, such as for example the AMAXA CELL LINE NUCLEOFECTOR™ system (Amaxa Biosystems Inc., Nattermannallee, Germany; www-amaxa-com), have been customized to transduce specific types of eukaryotic cells. In an embodiment, CD36⁺ erythroid progenitor cells are transfected using NUCLEOFECTOR™ reagent V and program T-19 on the NUCLEOFECTOR™ device according to the manufacturer's instructions (Amaxa Biosystems Inc., Nattermannallee, Germany). In another embodiment, CD36⁺ erythroid progenitor cells are transfected using NUCLEOFECTOR™ reagent R and program T-20 or V-001. In another embodiment, CD36⁺ erythroid progenitor cells are transfected using NUCLEOFECTOR™ reagent monocyte cell and program Y-001. In another embodiment, CD36⁺ erythroid progenitor cells are transfected using NUCLEOFECTOR™ reagent CD34 progenitor cells and program U-08.

In some embodiments, the viral stock can be diluted. Typically, viral stocks include about 10¹² to 10¹³ genomes/ml. Viral stocks can be diluted from about 10⁻³ to about 10⁻¹⁰ fold. In some embodiments, the virus can be diluted to about 10⁻⁸ and virus replication can still be detected in permissive cells such as the CD36+ cells described herein.

The cells can be incubated in culture medium following contact with infectious parvovirus B19 or introduction of the vector comprising a B19 genome. In an embodiment, cells infected with B19 are incubated at 4° C. for 2 hours to allow for viral attachment to the cell. In some embodiments, the unattached virus is removed from the culture after the attachment period. In some embodiments, the unattached virus is not removed from the culture. Transfected cells can be plated in culture medium immediately following transfection. The cells may be incubated for about 10 min to about 30 min at about 25° C. to about 37° C., more preferably about 30° C. to about 37° C., more preferably 37° C. before plating the cells. Once plated, the cells are incubated under conditions sufficient to provide for production of viral genomes. In some embodiments, the infected cells or transfected cells are incubated at 37° C. for about 2 to about 4 hours, more preferably at least about 6 hours, more preferably at least about 12 hours, more preferably at least about 18 hours, more preferably at least about 24 hours and more preferably up to 48 hours. In an embodiment, the infected or transfected cells are incubated for about 48 hours. In some embodiments, the infected or transfected cells are incubated for about one to five days or even up to 7 days. Infectious virus particles can be isolated or recovered from supernatants or cell lysates. In an embodiment, B19 is harvested from the supernatant of the infected cells.

To determine if B19 virus produced by the methods of the invention is infectious, supernatants prepared from infected or transfected cells or cell lysates from infected or transfected cells can be used to infect non-infected or non-transfected eukaryotic cells. In an embodiment, the eukaryotic cells are permissive. Examples of permissive cells include, but are not limited to, primary erythroid progenitor cells from bone marrow, fetal liver and blood; megakaryoblast cells; UT7/Epo cells, UT7/Epo-S1 cells, KU812Ep6 cells, JK-1, MB-O2 and CD36⁺ erythroid progenitor cells. Other eukaryotic cell types may also be utilized including 293 cells, CHO cells, Cos cells, Hela cells, BHK cells, K562 and SF9 cells. In an embodiment, the non-infected or non-transfected cells are UT7/Epo-S1 cells or CD36⁺ erythroid progenitor cells.

In some embodiments, production of B19 viral genomes by the methods of the invention may be detected by analyzing the infected cells for B19 DNA. In some embodiments, an increase in viral DNA is detected. Methods for detecting B19 DNA include, but are not limited to, PCR and quantitative PCR (qPCR). In some embodiments, B19 infection can be determined by detection of B19 transcripts. In an embodiment, the spliced transcripts are spliced capsid transcripts encoding, for example, VP1 or VP2. In an embodiment, the spliced transcripts are alternatively spliced capsid transcripts encoding, for example, VP1 or VP2. The methods of detection include but are not limited to, PCR and quantitative PCR (qPCR).

In some embodiments, B19 infected cells can be detected by antibodies that specifically bind to B19 proteins, such as the capsid protein. In other embodiments, B19 infected cells can be identified by the presence of cytopathology. Methods for such detection are known to those of skill in the art.

In some embodiments, B19 infected cells can be identified by identifying differential regulation of one or more genes as shown in Table 15 or Table 16.

Production of infectious virus by infected permissive cells can be determined by infecting uninfected cells using supernatant from the infected cells or using the cell lysate of infected cells. In an embodiment, infectious B19 is detected by infecting cells with supernatant from the previously infected cells and analyzing the cells for B19 transcripts. In an embodiment, infectious B19 is detected by infecting cells with supernatant from the infected transformed cells and analyzing the cells for B19 transcripts. Detection of spliced capsid transcripts, NS transcripts, or other viral transcripts indicate that the parvovirus B19 is infectious. In an embodiment, detection of capsid transcripts or NS transcripts indicates the parvovirus B19 is infectious.

Production of infectious B19 virus can also be detected by analyzing the infected cells for B19 viral proteins. Detection of B19 capsid proteins indicates the parvovirus B19 is infectious. In an embodiment, the B19 viral proteins are capsid proteins, such as for example VP1 and VP2. In an embodiment, infectious parvovirus B19 virus is identified by contacting cells with supernatant from the transfected cells and analyzing the contacted cells for B19 viral proteins. In another embodiment, in vitro neutralization assays can be performed to test whether neutralizing monoclonal antibodies against parvovirus B19 capsids are able to block the infection caused by the cell lysates of transfected cells. Blocking of infectivity by neutralizing antibodies is one method to determine if the virus is infectious.

E. Diagnostic Methods

The disclosure provides for methods of diagnosis of B19 infected cells and/or B19 infection. In an embodiment the CD36+ erythroid progenitor cells (whether primary, secondary, or immortalized) are used to detect the presence of B19 infectious virus from a sample. The CD36+ cells may be frozen and thawed, and then cultured in expansion medium to provide a cell culture for detecting infectious B19 from a biological sample. Samples can include blood, tissue sample, urine, amniotic fluid, placental microvilli, cord blood, serum and the like.

In an embodiment, a method for detecting parvovirus B19, comprises contacting a CD 36⁺ erythroid progenitor cell with a sample and culturing the cell under conditions to provide for replication of parvovirus B19 genome. The CD36+ erythroid progenitor cell can be a primary, secondary, or immortalized cell, and may be frozen and then thawed. The CD36⁺ erythroid cells are cultured in expansion medium as described herein. In some embodiments, the CD 36+ erythroid cell population has at least 25% CD36, globoside or both positive cells. In some embodiments, the CD 36+ erythroid cell population is at least 25% to 100% of the cells are CD36+ and globoside+cells, and less than 70% of the cells are CD33⁺.

In some embodiments, the CD36+ erythroid cells are cultured for a 1, 2, 3, 4, 5, 6, 7, 8, 9, 10, and any number up to 350 days in culture. In some embodiments, the cells can be cultured indefinitely. In some embodiments, the CD36+ erythroid cells are cultured for at least 4 days before contact with the sample.

In an embodiment, after contact with the sample, the CD36+ cells are incubated for at least 3, 6, 12, 24, 48 hours or more. In some embodiments, the CD36+ cells are incubated for at least 6 to 48 hours. The virus can be incubated for at least 5 days and the culture can be continued for at least 7 days in the presence of fresh medium. The presence of B19 virus can be detected by a variety methods including, detecting viral DNA, viral transcripts, the presence of viral antigens using antibodies, using the supernatant to reinfect a permissive cell culture, and detecting cytopathology as described previously hereon. One or more of these methods may be used in conjunction with each other.

The invention also provides methods for screening for antagonists that may inhibit or antagonize B19 infection. Antagonists can include antibodies, antisense, si RNA, aptamers, and small molecule inhibitors. Some antibodies may be defined as neutralizing antibodies. In an embodiment, the method comprises contacting a sample comprising B19 with a candidate antagonist and administering the contacted B19 to cells of the invention. Candidate compounds that inhibit infection of the cells of the invention are identified as antagonists. The antagonist effect of a candidate antagonist is determined by analyzing cells for B19 capsid proteins or B19 transcripts as described above.

The invention also provides methods for screening for antibodies that may inhibit or antagonize B19 infection of the permissive cells of the invention. Some antibodies maybe defined as neutralizing antibodies. In an embodiment, the method comprises contacting a sample comprising B19 with a candidate antibody and administering the contacted B19 to cells of the invention. Candidate antibodies that inhibit infection of the cells of the invention are identified as antagonist antibodies. The antagonist effect of anti-B19 antibodies may be determined by analyzing cells for B19 capsid proteins or B19 transcripts as described above. Methods for producing antagonist antibodies are known. Antagonist antibodies can be prepared and screened for as described, for example, in U.S. 2006/0008469.

The invention can be used to identify infectious B19 virions. B19 has been known to produce 1 infectious particle in 10e3 to 10e5 particles. B19 DNA has also been known to persist for years after infection of an individual. Using CD36⁺ erythroid cells would determine the presence of infectious virions by the production of B19 transcripts or increasing DNA production.

In some embodiments, kits for diagnosis of B19 infection can include CD36+ erythroid progenitor cells and one or more of empty viral capsids, antibodies to B19 proteins such as capsid proteins, probes or primers for detecting B19 viral transcripts and B19 genomes. In some embodiments, the kit includes a B19 virus for comparison purposes. In some embodiments, the B19 virus is a viral clone in a replicable vector. In some embodiments, the kit comprises a composition comprising parvovirus B19 of at least about 10³ to 10¹⁰ genomes/ml, more preferably about 10³ to 10⁶ genomes/ml. The composition can then be diluted to provide for a consistent amount of virus to analyze each sample. Alternatively, the kit may contain about 10³ to 10¹⁰ virus particles or portions thereof in a composition or attached to an assay surface, excluding empty viral capsid.

Genes differentially expressed in viral infected cells can be utilized in diagnostic kits and methods for detection of B19 infected cells. The gene expression profile of one or more genes differentially regulated can be used to identify virus infected cells. Such genes can be selected from those provided in Table 15 and/or Table 16. Other markers of B19 infected cells include one or more of differentially expressed genes as shown in Table 15 or Table 16, comparing timepoint zero infection to any other timepoint (3, 6, 12, 24, and 48) hours post-infection. In some embodiments, the diagnostic assay or kit may include detecting 1, 2, 3, 4, 5, 6, 7, 8, 9, 10, and any number up to all of the 309 genes. Probes, primers, and antibodies for detecting the genes or gene products in Table 15 can readily obtained by those of skill in the art. It is understood in the art that a polynucleotide encoding a gene product can be represented by a number of different transcript sequences and/or detected using a number of different probes and/or primers. A number of different publicly available and fee based databases provide for information regarding those sequences and the availability of probes or primers for detecting any of the genes presented in Table 15 or 16. Such databases include the NCBI database, Unigene database, the IMAGE consortium, Affymetrix, Agilent, Invitrogen, and Genecards databases.

The reagents for detection include antibodies, probes, primer, reagents for assay of activity of the biomarker. Such methods are known to those of skill in the art and include ELISA, PCR, Immunofluorescence, western blots, southern blots, and microarray detection using oligonucleotides or antibodies. In some embodiments, the kit or microarray does not detect more than 400 different genes or ests. In some embodiments, the kit or microarray does not detect more than 400, 399, 398, 397 and any number down to at least 2 different genes. In some embodiments, the kit or microarray does not detect more than 400 different genes or ests and includes at least one an antibody or oligonucleotide for detecting a B19 transcript such as a capsid protein. In some embodiments, the kit or microarray does not detect more than 400 different genes or ests and includes at least one an antibody or oligonucleotide for detecting a B19 transcript such as a capsid protein or for detecting a viral genome for example by detecting at least one of the ITRs or the P6 promoter. In some embodiments, a kit comprises antibodies or oligonucleotides that bind to and detect all B19 viral transcripts and/or the viral genome, for example by detecting at least one of the ITRs or the P6 promoter.

Some of the genes differentially expressed may be detected as secreted products using antibodies or other assays, for example, Luminex technology as described at the Luminex web page. In other embodiments, the genes selected that are differentially expressed are increased or decreased at least two fold at 48 hours post infection.

In some embodiments, a kit or microarray may include oligonucleotides or antibodies for the detection of one or more of the following genes shown in Table 16. In some embodiments, the kit or microarray include one or more control or housekeeping genes. In some embodiments the kit or micrarray includes antibodies or oligonucleotides for detecting a B19 transcript, genome, or protein. In some embodiments, the kit or microarray does not include detecting more than 400 different genes or ests. In some embodiments, the kit includes a B19 virus for comparison purposes. In some embodiments, the kit or microarray does include an antibody or oligonucleotide for detecting a B19 transcript such as capsid protein such as VP1 or VP2. In some embodiments, the kit or microarray includes an antibody or oligonucleotide for at least one of the 1, 2, 3, 4, 5, 6, 7, 8, 9, 10, 11, 12, 13, and 14 of the genes shown in Table 16. The table below shows the top gene genes differentially expressed (fold increase or decrease) at timepoints 6 hours and 48 hours post-infection. The sequences and gI numbers for these genes are provided in Table 15.

TABLE 16

| Description | 6 hr PI |
|---|---|
| interleukin 8 | 2.225 |
| elastase 2, neutrophil | 1.831 |
| Nuclear factor I/A | 1.804 |
| myeloperoxidase | 1.792 |
| AV711904 DCA Homo sapiens cDNA clone DCAAIE08 5′, | 1.756 |
| mRNA sequence. | |
| Cytochrome P450, family 1, subfamily B, polypeptide 1 | 1.66 |
| ATP synthase, H+ transporting, mitochondrial F1 | 1.61 |
| complex, gamma polypeptide | |
| interferon-induced protein 44 | 1.598 |
| immediate early response 3 | 1.583 |
| interferon-induced protein with tetratricopeptide | 1.569 |
| repeats 1 | |
| Description | 48 hr PI |
| AV711904 DCA Homo sapiens cDNA clone DCAAIE08 5′, | 5.34 |
| mRNA sequence. | |
| Charcot-Leyden crystal protein | 4.35 |
| tachykinin 3 (neuromedin K, neurokinin beta) | 3.917 |
| cytochrome P450, family 1, subfamily B, polypeptide 1 | 3.833 |
| elastase 2, neutrophil | 3.638 |
| myeloperoxidase | 3.21 |
| myeloperoxidase | 3.124 |
| Cytochrome P450, family 1, subfamily B, polypeptide 1 | 3.121 |
| carboxypeptidase A3 (mast cell) | 2.952 |
| actin, alpha 2, smooth muscle, aorta | 2.944 |

In some embodiments, the methods for diagnosing or detecting and/or the kits include detecting one or more of the genes of Table 15 that have at least a two fold change in expression. In some embodiments the methods for diagnosing or detecting and/or the kits include detecting one or more of the genes: TGD (SEQ ID NO:121), MT1E (SEQ ID NO:278), NIP3 (SEQ ID NO:301), MT1 (SEQ ID NO:295), MT1 (SEQ ID NO:280), Car3 (SEQ ID NO:155), NF1A (SEQ ID NO:238), TGD (SEQ ID NO:129), (SEQ ID NO:251), NKB (SEQ ID NO:4), CALB1 (SEQ ID NO:67), COCH (SEQ ID NO:30), ATDC (SEQ ID NO:117), CALB (SEQ ID NO:89), HSP72 (SEQ ID NO:156), HSP72 (SEQ ID NO:175), c-fos (SEQ ID NO:159), NE (SEQ ID NO:5), AD2 (SEQ ID NO:94), IL-6 (SEQ ID NO:7), HSP70-2 (SEQ ID NO:221), AZU (SEQ ID NO:22), TOMM40 (SEQ ID NO:34), IBP2 (SEQ ID NO:120), IL-8 (SEQ ID NO:157), K60 (SEQ ID NO:114) or (SEQ ID NO:306), EV19 (SEQ ID NO:279), CSH1 (SEQ ID NO:183), MB2 (SEQ ID NO:97), GRO2 (SEQ ID NO:147), DEC1 (SEQ ID NO:277), SLC25A37 (SEQ ID NO:299) and combinations thereof.

F. Uses

Particles or clones produced by the methods and CD36⁺ erythroid progenitor cells of the invention can be utilized in a variety of assays and to develop therapeutic products. As discussed previously, a permissive cell line capable of producing useful quantities of B19 and methods for consistently obtaining significant amounts of infectious virus in cell culture were not readily available. An in vitro system for producing virus particles can be used in diagnostic methods to identify the presence of virus in a variety of diseases and disorders. An in vitro system for producing virus particles can be used in screening methods to identify agents such as antibodies or antisense molecules that can inhibit viral infectivity or reproduction. The virus particles and/or clones in a cell of the invention can be utilized to form immunogenic compositions to prepare therapeutic antibodies or vaccine components. Antibodies and primers can be developed to specifically identify different parvovirus B19 isolates. The ability to produce virus particles consistently in vitro is also useful to produce attenuated virus that may be used in a vaccine.

Parvovirus B19 particles or B19 clones and CD36+ erythroid cells produced by the methods and cell line of the invention are useful in diagnostic assays and kits. The presence or absence of an antibody in a biological sample that binds to a B19 clone produced by the methods and cells of the invention can be determined using standard methods. In an embodiment, the diagnostic assay kit is a serological assay kit that contains B19 particles produced by the method and cells of the invention. Such an assay kit will be sensitive and cost effective because using the entire virus will allow for detection of antibodies to epitopes as presented by naturally occurring virus.

The B19 particles and/or clones of the invention are also useful to produce antibodies to parvovirus B19. The antibodies are useful in diagnostic assays for detecting the presence of parvovirus B19 virus particles in a biological sample. Methods for producing antibodies are known. Antibodies to B19 and methods for developing antibodies to B19 are described, for example, in U.S. 2006/0008469. Antibodies are useful in diagnostic assays, and to develop therapeutics.

The invention also provides methods for screening for antibodies that may inhibit or antagonize B19 infection of the permissive cells of the invention. Some antibodies maybe defined as neutralizing antibodies. In an embodiment, the method comprises contacting a sample comprising B19 with a candidate antibody and administering the contacted B19 to cells of the invention. Candidate antibodies that inhibit infection of the cells of the invention are identified as antagonist antibodies. The antagonist effect of anti-B19 antibodies may determined by analyzing cells for B19 capsid proteins or B19 transcripts as described above. Methods for producing antagonist antibodies are known. Antagonist antibodies can be prepared and screened for as described, for example, in U.S. 2006/0008469.

The invention can be used to identify infectious B19 virions. B19 has been known to produce 1 infectious particle in 10e3 to 10e5 particles. B19 DNA has also been known to persist for years after infection of an individual. Using CD36⁺ cells would determine the presence of infectious virions by the production of B19 transcripts of increasing DNA production.

Infectious B19 produced by the methods and cells of the invention can be used as immunogenic compositions to prepare vaccine components and/or to develop antibodies that can be used in diagnostic or other assays. For example, cells of the invention comprising B19 virus particles and/or clone can be heat inactivated and used as an immunogen. Passaging of a virus particle and/or clone in cells of the invention can provide an attenuated strain of B19 useful in vaccine compositions. In some embodiments, the immunogenic composition comprises at least about 10³ to about 10¹⁰ viral genomes or viral particles/ml. A vaccine against B19 would be useful, for example, for preventing B19 associated diseases and treating patients with hereditary anemias, such as sickle cell anemia, who are susceptible to transient aplastic crises, seronegative pregnant women who are at risk for hydrops fetalis, and immunocompromised individuals at risk for persistent infection and chronic red cell aplasia.

Genes differentially expressed in viral infected cells can be utilized in diagnostic kits and methods for detection of B19 infected cells. The gene expression profile of one or more genes differentially regulated can be used to identify virus infected cells. Such genes can be selected from those provided in Table 15. The reagents for detection include antibodies, probes, primer, reagents for assay of activity of the biomarker. Such methods are known to those of skill in the art and include ELISA, PCR, Immunofluorescence, western blots, southern blots, and microarray detection using oligonucleotides or antibodies. Other markers of B19 infected cells include one or more of differentially expressed genes as shown in Table 15, comparing timepoint zero infection to any other timepoint (3, 6, 12, 24, and 48) hours post-infection. In some embodiments, the diagnostic assay or kit may include detecting 1, 2, 3, 4, 5, 6, 7, 8, 9, 10, and up to all of the 309 genes. In some embodiments, the kit or microarray do not include more than 400 different antibodies or oligonucleotides. In some embodiments, the kit or microarray do not include more than 400 different antibodies or oligonucleotides and does include an antibody or oligonucleotide for detecting the B19 capsid protein.

Some of the genes differentially expressed may be detected as secreted products. In other embodiments, the genes selected that are differentially expressed are increased or decreased at least two fold at 48 hours post infection.

In some embodiments, a kit or microarray may include oligonucleotides or antibodies for the detection of one or more of the following genes shown in Table 16. In some embodiments, the kit or microarray include one or more control or housekeeping genes. In some embodiments the kit or micrarray include antibodies or oligonucleotides for detecting the B19 transcript or proteins. In some embodiments, the kit or microarray do not include more than 400 different antibodies or oligonucleotides. In some embodiments, the kit or microarray do not include more than 400 different antibodies or oligonucleotides and does include an antibody or oligonucleotide for detecting a B19 capsid protein such as VP1 or VP2. In some embodiments, the kit or microarray includes an antibody or oligonucleotide for at least one of 1, 2, 3, 4, 5, 6, 7, 8, 9, 10, 11, 12, 13, and 14 of the genes shown in Table 16. The table below shows the top gene genes differentially expressed (fold increase or decrease) at timepoints 6 hours and 48 hours post-infection.

G. Production of Antibodies

1. Polyclonal Antibodies

Polyclonal antibodies to B19 produced by the cells and methods of the invention are preferably raised in animals by multiple subcutaneous (sc) or intraperitoneal (ip) injections of the relevant antigen and an adjuvant. The relevant antigen may be, for example, one or more B19 clones produced by the cells and methods of the invention or one or more B19 proteins, such as NS, VP1, VP2, 11-kDa protein, 7.5-kDa protein, and/or protein X, derived from an infectious clone produced by the cells and methods of the invention or virus particle such as those produced by the methods as described herein. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfa succinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ½ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

In an alternative embodiment, the animals are immunized with a recombinant vector expressing one or more viral proteins derived from an infectious particle or clone produced by the cells or methods of the invention, such as for example VP1 and/or VP2, followed by booster immunizations with the viral proteins.

The polyclonal antibodies generated by the immunizations may undergo a screen for B19 antagonist activity. Preferably, antibodies to a B19 virus particle and/or clone inhibit the negative effect of B19 on erythrocyte production. In an embodiment, antibodies that specifically bind a B19 virus particle and/or clone encoded by a polynucleotide comprising a nucleic acid sequence of SEQ ID NO:1 inhibits infection of permissive cells.

The polyclonal antibodies are also screened by enzyme-linked immunoabsorbent assay (ELISA) to characterize binding. The antigen panel includes NS, VP1, VP2, 11-kDa protein, 7.5-kDa protein, protein X, and virus particles. Animals with sera samples that test positive for binding to one or more experimental antigens in the panel are candidates for use in monoclonal antibody production. The criteria for selection for monoclonal antibody production is based on a number of factors including, but not limited to, binding patterns against a panel of B19 viral proteins.

2. Monoclonal Antibodies

Monoclonal antibodies to a B19 produced by the cells and methods of the invention may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster or macaque monkey, is immunized as described above to elicit lymphocytes that produce or are capable of producing antibodies that will specifically bind to a B19 particle and/or clone or viral proteins derived from a B19 particle and/or clone used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells are than seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen and HIV Env. Preferably, the binding specificity of monoclonal antibodies produced by hybridoma cells is determined by immunoprecipitation or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

The monoclonal antibodies are characterized for specificity of binding using assays as described previously. Antibodies can also be screened for antagonist activity as described previously.

3. Human or Humanized Antibodies

Humanized forms of non-human (e.g., murine) antibodies are chimeric antibodies that contain minimal sequence derived from non-human immunoglobulin. For the most part, humanized antibodies are human immunoglobulins (recipient antibody) in which residues from a CDR of the recipient are replaced by residues from a CDR of a non-human species (donor antibody) such as mouse, rat, rabbit or nonhuman primate having the desired specificity, affinity, and capacity. Useful non-human antibodies are monoclonal antibodies that bind specifically to parvovirus B19. Useful non-human antibodies also include antibodies that inhibit B19 infection of permissive cells. In some instances, framework region (FR) residues of the human immunoglobulin are replaced by corresponding non-human residues. Furthermore, humanized antibodies may comprise residues that are not found in the recipient antibody or the donor antibody. These modifications may be made to improve antibody affinity or functional activity. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech 5:428-433 (1994).

Human antibodies that specifically bind and/or antagonize parvovirus B19 can also be made using the transgenic mice available for this purpose or through use of phage display techniques.

An in vitro system for producing infectious virus particles comprising the cells and methods of the invention can be used in screening methods to identify agents such as antibodies or antisense molecules that can inhibit viral infectivity or reproduction. A screening method comprises introducing the viral genome of an infectious particle and/or clone of parvovirus B19 into a cell of the invention, contacting the cells with a potential inhibitory agent, and determining whether the inhibitory agent inhibits infectivity or replication of the viral genome in the cells. Methods for detecting infectivity and replication of the viral genome have been described herein. Potential inhibitory agents include antibodies and anti sense molecules.

The ability to produce infectious parvovirus particles in vitro by the cells and methods of the invention allow for the development of a vaccine or vaccine components. A vaccine can be comprised of heat inactivated virus or attenuated virus. Inactivated virus particles can be prepared from production of infectious clones and/or particles using methods known to those of skill in the art. Attenuated virus can be obtained by serially passaging the virus under conditions that make the virus non pathological to humans. The attenuated virus is preferably passaged through a cell and under certain conditions that provide for an altered virus that is less pathological to humans. Vaccine components can also include one or more of the parvovirus proteins or parvovirus proteins combined with epitopes from other infectious agents.

The following examples are provided for illustrative purposes only, and are in no way intended to limit the scope of the present disclosure.

### EXAMPLES

### Example 1

### Erythroid Progenitor Cells Derived from CD34⁺G-CSF Mobilized Peripheral Blood Stem Cells (PBSC) are Permissive for Parvovirus B19 Infection and Produce Increased Amounts of Parvovirus B19 Compared to UT-7/Epo-S1 Cells

Parvovirus B19 (B19) is highly erythrotopic and replicates in erythroid progenitor cells found in bone marrow or fetal liver. A limited number of cell lines support B19 replication in vitro. Previous studies have shown that UT-7/Epo-S1 cells, a subclone of a megakaryoblastoid cell line with erythroid characteristics, to be one of the most permissive cell lines (Wong, et. al., 2006, Journal of Clinical Virology, 35:407-413). These cells, however, are only semi-permissive with limited replication of B19.

Methods for producing mature erythrocytes from CD34 hematopoietic stem cells have been reported (Giarratana et al., 2005, Nature Biotech., 23:69-74). This example describes a method for culturing CD36⁺ erythroid progenitor cells that are permissive to B19 infection from CD34+ or CD133+ hematopoietic stem cells. Recently, CD133 (formerly AC133) has been used to isolate hematopoietic stem cells and progenitor cells. CD133 has been used as a selective marker for immature hemtopoietic stem cell and progenitors.

### Methods

Cell Culture. Human CD34⁺ cells were isolated from G-CSF mobilized peripheral blood stem cells from normal donors by purification using the Baxter Isolex 300i Magnetic Cell Selection System. Human CD133⁺ cells were isolated from G-CSF mobilized peripheral blood stem cells from normal donors by purification using the Milotenyi Magnetic Cell Selection System. Prior to expansion and if necessary, the cells were cultured in maintenance media (BIT 9500 medium (StemCell Tech. Inc., Vancouver, British Columbia) diluted 1:5 in AMEM (Mediatech Inc., Herndon, Va.) and supplemented with 900 ng/ml ferrous sulfate (Sigma-Aldrich, St. Louis, Mo.) and 90 ng/ml ferric nitrate (Sigma-Aldrich)) and cultured in the maintenance media at 37° C. in 5% CO₂ for 4 days. The maintenance media had a final concentration of 10 mg/ml deionized BSA, 10 μg/ml recombinant human insulin, and 200 μg/ml iron saturated human transferrin.

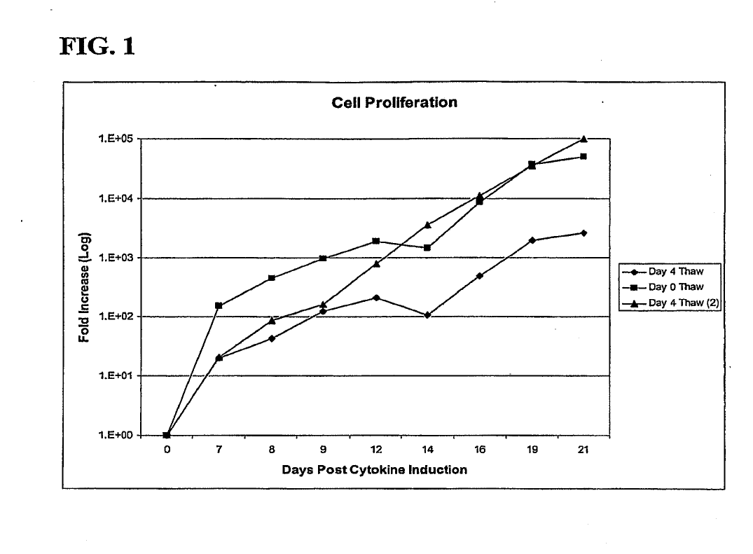

Cell proliferation and erythroid differentiation was induced as follows. Approximate 1×10⁴ cells/mL were cultured in expansion media (maintenance media diluted 1:5 in AMEM and supplemented with 10⁻⁶M hydrocortisone, 5 ng/mL human IL-3 (R&D Systems, Minneapolis, Minn.) 100 ng/ml recombinant human stem cell factor (StemCell Tech. Inc., Vancouver, British Columbia), 3 IU/ml recombinant human erythropoietin (Amgen, Thousand Oaks, Calif.), 900 ng/ml ferrous sulfate (Sigma-Aldrich, St. Louis, Mo.) and 90 ng/ml ferric nitrate (Sigma-Aldrich)) at 37° C. with 5% CO₂ in air for 4 days and then the culture volume was expanded 1:5 in maintenance media for an additional 4 days. Once the cell density reached approximately 1−2×10⁶ cells/mL, cells were reduced to a concentration of about 1−5×10⁵ cells/mL. This allowed the culture to be maintained in an environment whereby the cytokines and growth factors would not be depleted. Cells were enumerated daily and would typically expand 3 to 5 logs within 21 days. (FIG. 1).

At days 1, 4, and 8 of cell culture in the expansion media, cells were sampled and analyzed for cell surface antigens by FACS. Approximately 5×10⁵ cells in a volume of 100 μl were centrifuged, washed with fresh AMEM, stained with 5 μl anti-CD36 FITC antibodies for 30 min. on ice, washed with AMEM, resuspended in 500 μl AMEM, and analyzed by FACS using the Beckman Coulter Cytomics FC500. Cells were also collected onto glass slides by cytocentrifugation (1500 rpm, 8 min.), fixed in methanol-acetone (1:1, −20° C.), stained with FITC, propidium iodide or DAPI, and observed under UV microscopy.

The megakaryoblastoid cell line UT-7/Epo-S1 was used as a comparative control for B19 infection (Shimomura et al., 1993, Virology, 194:149-156; Shimomura et al., 1992, Blood, 79:18-24, Wong, et. al., 2006, Journal of Clinical Virology, 35:407-413). The UT-7/Epo-S1 cells were cultured as previously described (Shimomura et al., 1993, Virology, 194:149-156; Shimomura et al., 1992, Blood, 79:18-24, Wong, et. al., 2006, Journal of Clinical Virology, 35:407-413). UT-7/Epo-S1 are megakaryocytes and most of the cells in the population express CD33 on the cell surface. Briefly, UT-7/Epo-S1 cells were cultured in Iscove's modified Dulbecco's medium (IMDM) supplemented with 10% fetal bovine serum, antibiotics, and 2 U/ml recombinant human erythropoietin (Amgen, Thousand Oaks, Calif.) at 37° C. in 5% CO₂.

To determine necessity for the expansion media cytokine cocktail, the erythroid progenitor cells and UT7/Epo-S1 cells were cultured in the same media used to culture UT7/Epo-S1 cells (IMDM supplemented with 2 IU EPO/mL) and also in IMDM media supplemented with 50 ng/mL rhuIL-3 and 5 IU rhuEPO/mL which is a media that typically used for culturing bone marrow cells. To determine if the expansion media would render UT7/Epo-S1 more permissive to B19 infection, these cells were also cultured in the expansion media.

Infection Assay. High titer B19 virus was obtained from different sources (Wong, et. al., 2006, Journal of Clinical Virology, 35:407-413). One source of parvovirus B19 (J35) was obtained from the serum of a child with sickle cell anemia undergoing aplastic crisis and sent to NIH for diagnostic purposes. This serum was found by dot blot assay (Nguyen et al., 2002, Virology, 301:374-380) to contain approximately 10¹³ genome copies of B19/ml. Viral stocks V1 and V2 were obtain from normal donors provided to us by Aris Lazo at V.I. Technologies (Watertown, Mass.). At day 8, cells were infected with the various dilutions of V1 serum containing 2×10¹² genome copies of B19/mL. In 96 well plates, 2×10⁴ cells were infected with 10 μl of serially diluted B19. The cells were incubated for 2 hr at 4° C. and then expanded with 80 μl of expansion media and incubated at 37° C. in 5% CO₂. In some cases, the infection assay is sealed up proportionately.

At different times post infection (from day 0 to day 5), DNA or RNA was extracted from infected CD36⁺ erythroid progenitor cells and UT7/Epo-S1 cells by QIAmp DNA mini Kit (Qiagen, Valencia, Calif.) or the RNEasy Micro Kit (Qiagen). Quantitative real-time PCR (qPCR), using the primers and probes shown in Table 3, was carried out using a Quantitect Probe PCR Kit (Qiagen) to detect B19 viral DNA Most of the reporters (6-FAM™, HEX™, TET™, Cy3™, Cy5™, JOE, etc.) and quenchers (TAMRA™, Iowa Black™, BHQ1®, BHQ2®, etc.) combinations can be used on the probes.

TABLE 3

SEQ

| | | |
|---|---|---|
| DNA | Primer/ | ID |

| region | Probe | Nucleotide Sequence | NO: |
|---|---|---|---|
| J35- | Forward | 5′ TACCTGTCTGGATTGCAAAGC 3′ | 309 |
| 2591F | | | |
| Capsid | | | |
| J35- | Reverse | 5′ GATGGGTTTTCTAGGGGATTATC 3′ | |
| 2591F | | | |
| Capsid | | | |
| J35 | Probe | 5′ 6-FAM-ATG GTG GGA AAG TGA | 311 |
| | | TGA TGA ATT TGC TA-3′BHQ | |

At different times post infection (from day 0 to day 5), RNA was extracted from infected CD36⁺ erythroid progenitor cells and UT7/Epo-S1 cells using GeneStrip™ System (RNAture, Irvine, Calif., USA, now Qiagen TurboCapture), followed by synthesis of the corresponding cDNA using 500 ng (5 μl from 100 ng/μl) of random primers (Invitrogen, Carlsbad, Calif., USA) and M-MLV RT Polymerase (Invitrogen) or Superscript II Reverse Transcriptase (Invitrogen) in a final volume of 50 μl. The cDNA samples were used for RT-PCR and quantitative real-time RT-PCR (qRT-PCR) assays. The RT-PCR reaction was carried out as previously described in Nguyen et al., 2002, Virology, 301:374-380 and amplicons were visualized by gel electrophoresis (2.5% NuSieve agarose gel). The qRT-PCR assays were performed as described above. The cDNA samples were amplified for capsid and NS transcripts for B19 and (3-actin, a housekeeping gene, using the primers and probes shown in Table 4.

TABLE 4

| | | | SEQ |
|---|---|---|---|
| | Primer/ | | ID |
| Transcript | Probe | Nucleotide Sequence | NO: |
| Capsid | Forward | 5′ CCTGGGCAAGTTAGCGTAC 3′ | 312 |
| | Reverse | 5′ ATGATCCTTGCAGCACTGTCA 3′ | 313 |
| | Probe | FAM-TATGTTGGGCCTGGCAA-BHQ1 | 314 |
| NS | Forward | 5′ GTTTTATGGGCCGCCAAGTA 3′ | 315 |
| | Reverse | 5′ ATCCCAGACCACCAAGCTTTT 3′ | 316 |
| | Probe | 5′ 6-FAM- | 317 |
| | | CCATTGCTAAAAGTGTTCCA-3′BHQ1 | |
| β-actin | Forward | 5′ GGCACCCAGCACAATGAAG 3′ | 318 |
| | Reverse | 5′ GCCGATCCACACGGAGTACT 3′ | 319 |
| | Probe | 5′ JOE- | 320 |
| | | TCAAGATCATTGCTCCTCCTGAGC | |
| | | GC-3′BHQ | |

Quantitation of each amplicon was performed by interpolation with the respective standard curve to each target (NS, CP, β-actin) constructed with serial dilutions of the correspondent plasmid.

At different times post infection (from day 0 until day 5), cells were cytocentrifuged (1500 rpm for 8 min in a Shandon cytospin 4 cytocentrifuge) onto glass slides. The cells were fixed in acetone:methanol (1:1) at −20° C. for 5 min, washed twice in phosphate buffered saline (PBS) containing 0.1% fetal bovine serum, and incubated with a murine anti-B19 capsid protein monoclonal antibody (521-5D, gift of Larry Anderson, CDC) in PBS with 10% fetal calf serum for 1 hr at 37° C. After washing the slides twice in PBS, the slides were incubated with fluorescein isothiocyanate (FITC)-labeled goat anti-mouse IgG antibody (Jackson ImmunoResearch Laboratories, Inc., West Grove, Pa.) in PBS with 10% fetal calf serum and counterstained with Evans Blue for 30 mins at 37° C., washed in PBS, and examined by UV microscopy.

### Results

We observed the expansion of the erythroid progenitor cells in culture starting with CD34+ cells from day 0 up to day 26. Maximum growth of the CD36⁺ erythroid progenitor cells in the expansion medium was observed between days 0 and 21 of culture (FIG. 1). Cell mortality was less than 5%.

If cells were provided fresh media and maintained at <2×10e6 cells/mL, we observed exponential growth between day 4 and 21. Flow cytometry analysis confirmed that the CD36⁺ cells were expressing erythroid lineage markers CD36 and glycophorin A (GpA) and most importantly expressing the B19 receptor, globoside, but not CD34 on the cell surface, whereas the parent CD34+ cells did not express CD36, GpA or globoside. (Table 5, Table 6). Flow cytometry analysis confirmed the purity and the complete differentiation of the CD34⁺ cells into CD36⁺ cells. At days 8, the cells were non-enucleated, positive for globoside on the cell surface, and in some cases, visibly red, indicating the presence of hemoglobin. *NT means “not tested”

TABLE 5

| | |
|---|---|
| Days in | Cell Surface Antigens (% positive cells) |

| Culture | CD34⁺ | CD3⁺ | CD19⁺ | CD36⁺ |
|---|---|---|---|---|
| pre-culture | 76 | 2 | 5 | NT |
| 1 | NT | NT | NT | 20 |
| 4 | NT | NT | NT | 73 |
| 8 | 1.2 | 0 | 0 | 97 |

TABLE 6

| Cell Surface | Day in culture (percentage of positive cells) | | | | | | | | |
|---|---|---|---|---|---|---|---|---|---|
| Antigens | 0 | 1 | 2 | 3 | 4 | 5 | 6 | 7 | 8 |
| Sample G | | | | | | | | | |
| Globoside | 5 | 9 | 28 | 39 | 39 | 59 | 79 | 85 | 90 |
| CD36 | 2 | 6 | 15 | 31 | 45 | 73 | 80 | 88 | 95 |
| CD34 | 80 | 95 | 87 | 74 | 18 | 1 | 0 | 1 | 0 |
| Sample H | | | | | | | | | |
| Globoside | 2 | 4 | 16 | 34 | 36 | 36 | 49 | 58 | 70 |
| CD36 | 2 | 3 | 5 | 15 | 24 | 35 | 50 | 60 | 64 |
| CD34 | 79 | Not Det. | 81 | 79 | 5 | 6 | 0 | 1 | 12 |
| Sample L | | | | | | | | | |
| Globoside | 6 | 2 | Not Done | Not Done | Not Done | 51 | 72 | 87 | Not Done |
| CD36 | 2 | 7 | Not Done | Not Done | Not Done | 63 | Not Det. | 88 | Not Done |
| CD34 | 74 | Not Det. | Not Done | Not Done | Not Done | 0 | 0 | 0 | Not Done |

Hematopoietic precursors can be identified by their cell-surface marker distribution (Morey & Fleming, 1992; Watt, Gilmore et al., 1987). CD36 is typically found on erythroid progenitor and megakaryocytic cells but appears earlier on cells in the erythroid lineage and has been defined as a marker for erythroid progenitor cells (Okumura, Tsuji et al., 1992b; de Wolf, Muller et al., 1994a). As shown in Table 7, during the maturation of erythroblasts, cells also begin to express CD71, the receptor for transferrin (Migliaccio, Di et al., 2002a), the serum iron-transport protein, and glycophorin A (Migliaccio, Di et al., 2002b). Analysis of the cells surface antigens of our CD34+ selected PBSC indicated an absence of CD36, GpA and most importantly the P antigen, the B19 cellular receptor. In the course of 4 days in culturing in expansion media, cells began to present CD36 on their cell surface and by Day 8, cells were primarily CD36+/GpA+/globoside+, but CD34−. Moreover, the UT7/Epo-S1 cells, the most permissive cellular system for in vitro B19 infection assay (Wong & Brown, 2006d) available at the time of this study, are also primarily CD36⁺/globoside+ and a subpopulation is GpA+.

TABLE 7
Flow cytometry analysis

Cell population

| Surface antigen | CD34+ | CD36+ | UT7/Epo-S1 | K562 |
|---|---|---|---|---|
| Glycophorin A | 0.3 | 63.9 | 26.6 | NT |
| Globoside | 4.7 | 99.1 | 57.6 | NT |
| KU80 | NT | 0.0 | 1.9 | 3.4 |
| CD10 | 0.0 | 0.0 | 0.0 | NT |
| CD19 | 0.1 | 0.0 | 0.0 | NT |
| CD2 | 0.9 | 0.0 | 0.0 | NT |
| CD3 | 0.1 | 0.0 | 0.0 | NT |
| CD33 | 46.2 | 58.6 | 91.3 | NT |
| CD34 | 96.6 | 1.0 | 0.0 | NT |
| CD36 | 11.1 | 97.9 | 99.0 | NT |
| CD44 | 98.9 | 98.0 | 99.0 | NT |
| CD49e + CD29 | 99.0 | 59.0 | 58.8 | NT |
| CD71 | 48-63% | 97.0 | 96.5 | 96 |

*NT means “not tested”

We were able to generate a pure population of erythroid progenitors from PBSC whereby not only the most of the cells were CD36⁺, but nearly 100% of the cells were CD36⁺/CD34⁻ after culturing for 8 days in expansion media. As a result, the population of cells that was generated did not require further purification by immunomagnetic separation or by other means as typically described. In addition, this modified protocol allowed cells to continue to proliferate for up to 23-26 days after initial induction into the expansion media and the cells did not terminally differentiate into red blood cells. The CD36+ cells appeared to be directed toward terminal differentiation when the cell population reached >2×10⁶/mL without replenishment of fresh media. This may be caused by the depletion of cytokines and growth factors. Following this protocol, we established an in vitro erythropoiesis model from CD34+ hematopoeitic stem cells and generated a population of cells arresting at a specific stage of erythroid differentiation.

The initial infection study showed a greater amount of B19 transcript production in the CD36+ day 8 cells cultured in the expansion media as compared to CD36+ cells culture in IMDM with IL-3 and EPO and UT7/Epo-S1 cells cultured in the expansion media or in IMDM. CD36+ cells were 2-6% positive at infections using an inoculation at 10e6 ge/mL as compared to UT7/Epo-S1 cells which were able to detect the approximately the same percentage of positive cells at 10e9 ge/mL. (Table 8).

TABLE 8

| | CD36+ in EM | | | CD36+ in IMDM | | | UT7/Epo-S1 in EM | | | UT7/Epo-S1 in IMDM | | |
|---|---|---|---|---|---|---|---|---|---|---|---|---|
| ge/mL | CP/Actin | NS/Actin | IF | CP/Actin | NS/Actin | IF | CP/Actin | NS/Actin | IF | CP/Actin | NS/Actin | IF |
| 10⁹ | 369 | 349 | POS | 1,802 | 2,077 | 2-6% | 150 | 510 | 2-6% | 31 | 52 | 2-6% |
| 10⁸ | 1,428 | 1,902 | POS | 167 | 146 | NEG | 6 | 141 | NEG | 4 | 5 | 1 cell |
| 10⁷ | 2,501 | 4,414 | POS | 7 | 6 | NEG | 0 | 0 | NEG | 1 | 2 | NEG |
| 10⁶ | 85 | 137 | 2-6% | 1 | 0 | NEG | 0 | 0 | NEG | 0 | 0 | NEG |
| No V | 0 | 0 | NEG | N/A | N/A | NEG | 0 | 0 | NEG | 0 | N/A | NEG |

*CP/Actin and NS/Actin given as copies/μL

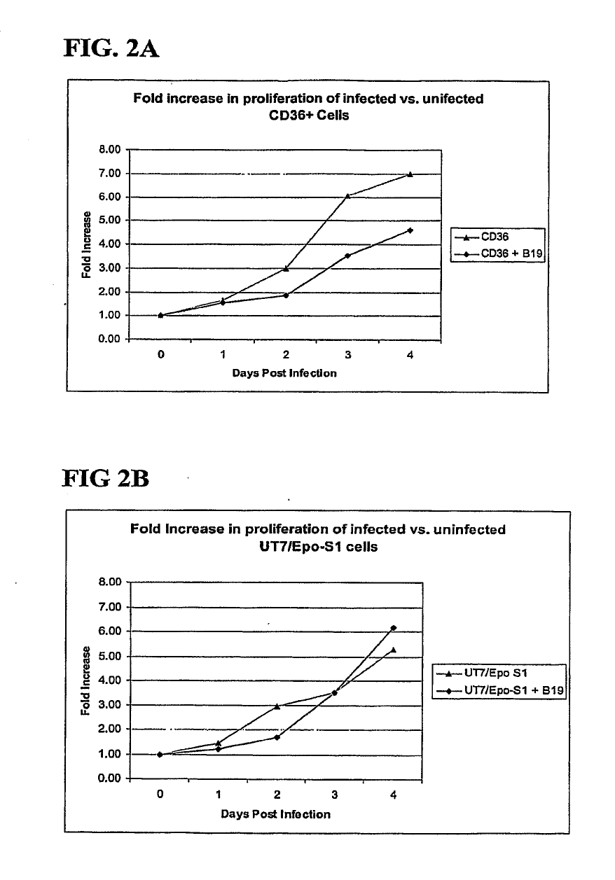

Therefore, by immunofluorescence, CD36+ cells were 3-logs more sensitive to infection compared to UT7/Epo-S1 cells. CD36+ cells infected with high titers of B19 seem to be undergoing morphological changes and cell death indicating a cytopathic affect. To determine if B19 affected cell proliferation, UT7/Epo-S1 and CD36+ cells were infected with 10⁷ ge/mL, of B19 and cell proliferation was monitored between the uninfected and infected cells (FIGS. 2a and 2b). CD36+ cells infected with B19 proliferated significantly less than uninfected cells. UT7/Epo-S1 cell proliferation did not seem affected by B19 which is consistent with previous observations.

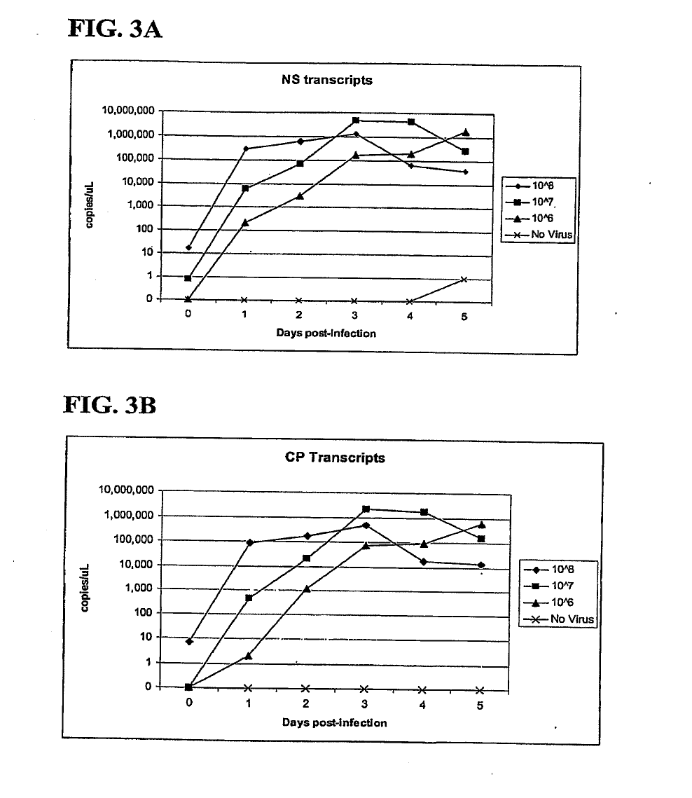

CD 36⁺ cells were analyzed for their permissiveness to B19 infection at day 8 and day 15 and shown to have similar transcript production levels (data not shown). Consequently, experiments were conducted using predominantly CD36⁺ day 8 cells as it seemed that cells were differentiated and amply proliferated. We compared the infection assays performed with serial dilutions of virus and analyzed the NS and capsid RNA transcripts at different times post infection. Transcripts can be readily detected at day 3 at a variety of viral inputs as shown in FIGS. 3A and 3B.

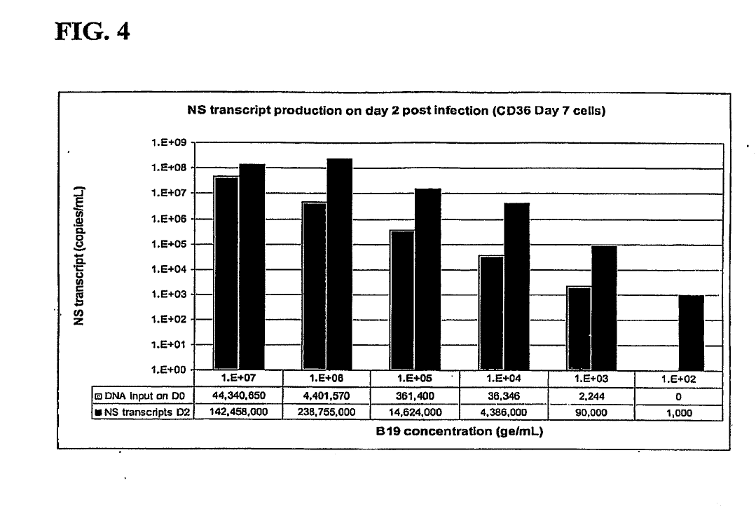

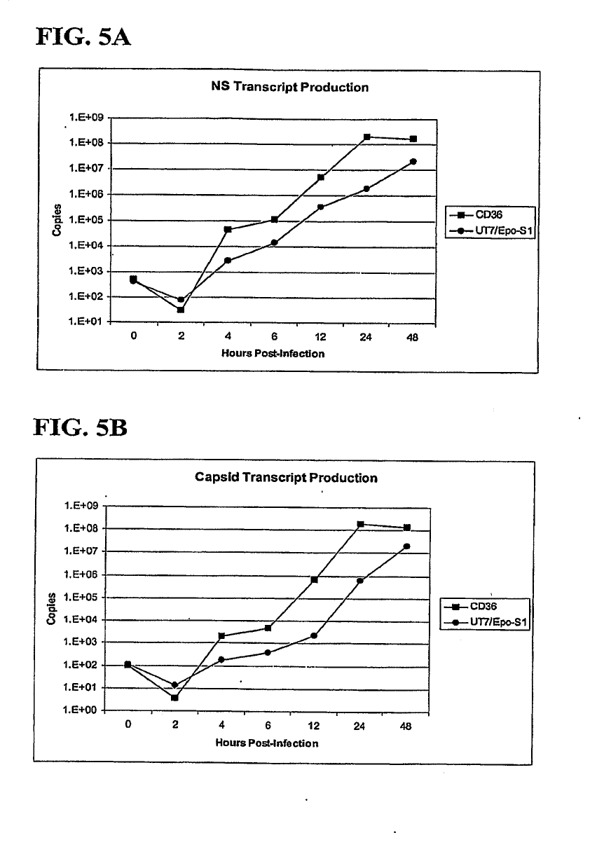

Using the same virus stock as for infection with UT7/Epo cells in determining the sensitivity to B19 infection (Wong & Brown, 2006f), CD36+ cells were able to detect as little as one infectious virus particle in 10e3 viral genome equivalents in plasma sample V1 as compared to UT7/Epo-S1 which detected one infectious virus particle in 10e5 (FIG. 4). In addition, CD36+ cells generally produce 1-2 logs more transcripts than UT7/Epo-S1 cells (FIG. 5A-NS transcripts and FIG. 5B-Capsid transcripts).

After 8 days of culture in expansion media, cells were analyzed for permissiveness to B19 infection. Permissiveness of the CD36⁺ erythroid progenitor cells for B19 replication was compared to UT7/Epo-S1 cells using qPCR. As shown in Table 9, DNA production was greatest 3 days post infection as detected by qPCR. Compared to UT7/Epo-S1 cells infected with B19 (10 dilution of viral stock having 2×10¹² genomes/ml or 2×10⁸ genomes/ml), there was an approximate 200 fold increase in viral DNA production in CD36⁺ erythroid progenitor cells 3 days post infection (10⁻⁴ dilution of the viral stock).

TABLE 9*

| B19 Dilution | | | | |
|---|---|---|---|---|
| 10⁻³ | 10⁻⁴ | 10⁻⁵ | 10⁻⁶ | 10⁻⁷ |

CD36+ Cells

| | | | | | |
|---|---|---|---|---|---|
| Day | 7,392,250 | 1,087,250 | 115,575 | 14,340 | 1,688 |
| 0 | | | | | |
| Day | 31,870,000 | 1,494,750 | 179,800 | 17,360 | 55,987 |
| 1 | | | | | |
| Day | 261,625,000 | 49,755,000 | 1,777,250 | 165,400 | 6,516 |
| 2 | | | | | |
| Day | 855,600,000 | 4,220,750,000 | 162,450,000 | 2,412,750 | 53,933 |
| 3 | | | | | |

UT7/Epo-S1 Cells

| | | | | | |
|---|---|---|---|---|---|
| Day | 10,527,500 | 1,168,500 | 167,975 | 18,378 | 1,745 |
| 0 | | | | | |
| Day | 18,127,500 | 1,446,250 | 155,825 | 12,973 | 1,835 |
| 1 | | | | | |
| Day | 61,150,000 | 4,532,250 | 559,425 | 24,430 | 7,278 |
| 2 | | | | | |
| Day | 34,982,500 | 19,615,000 | 2,079,000 | 25,683 | 1,782 |
| 3 | | | | | |

•Results in Table 9 are given in qenome equivalents (ge)/μl.

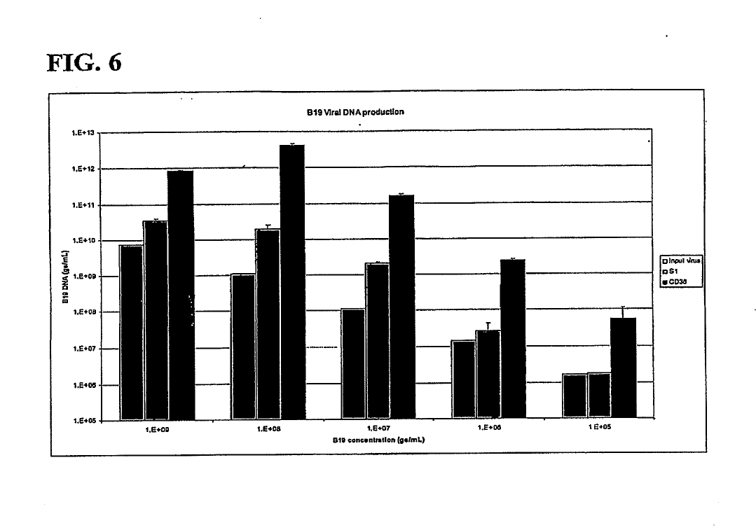

To compare the viral DNA production between the UT7/Epo-S1 cells and the CD36⁺ cells, serial dilutions of B19 containing plasma were used to infect cells and quantitative PCR (qPCR) was performed. As a result, an increase of viral DNA of up to 3.5 logs over input viral DNA was found in CD36⁺ cells whereas 1 log or less was seen in UT7/Epo-S1 and the greatest increase was seen with inoculation of virus between 10⁷ and 10⁸ ge/mL. Increases in viral DNA production can be seen even with an inoculation of virus at 10⁵ ge/mL (FIG. 6). B19 transcripts can typically be detected in inoculations at 10e3 ge/mL (data not shown).

NS and capsid transcripts from infected cells were quantitated by RT-PCR. As shown in Table 9, the CD36⁺ erythroid progenitor cells have similar sensitivity to B19 infections as UT7/Epo-S1 cells. As shown in Table 10, NS and capsid transcripts were significantly higher in CD36⁺ erythroid progenitor cells than UT7/Epo-S1 cells 4 hr to 48 hr post infection. The B19 stock in Table 10 had 2×10⁻² genome equivalents (ge)/ml. The results in Table 10 are given in copies/ml.

TABLE 10

| Hours post | CD36⁺ Erythroid | | | |
|---|---|---|---|---|
| B19 | Progenitor Cells | | UT7/Epo-S1 Cells | |
| infection | NS | CP | NS | CP |
| NoV | 1 | 0 | 4 | None Detected |
| 0 | 521 | 100 | 422 | 116 |
| 2 | 30 | 4 | 74 | 13 |
| 4 | 44,670 | 2,080 | 2,759 | 182 |
| 6 | 113,800 | 4,549 | 13,655 | 393 |
| 12 | 4,961,000 | 670,650 | 354,800 | 2,182 |
| 24 | 197,100,000 | 184,800,000 | 1,853,500 | 637,450 |
| 48 | 155,400,000 | 141,700,000 | 21,150,000 | 20,345,000 |

Transcripts corresponding to actin indicated the similar numbers of CD36⁺ erythroid progenitor cells and UT7/Epo-S1 cells were assayed (data not shown).

To confirm cells infected with parvovirus B19 were producing infectious virus particles, naïve CD36⁺ erythroid progenitor cells were infected with supernatants from infected CD36⁺ erythroid progenitor cells. The naïve cells were incubated with the supernatants (initial MOI of 100) for 2 hr and then washed with expansion media and incubated as described above. At day 0 to 3 post infection, capsid RNA transcripts were detected in the naïve cells infected with supernatants. As shown in Table 11, a small number of viral genomes (approximately 370 to 390 genomes/μl) were detected in the supernatants of the infected naive cells at day 0 and day 1 post infection. These genomes likely represent virus carried over from the washing step or virus particles that have detached from the surface of the naïve cells. The number of genomes and capsid transcripts detected in supernatant harvested at day 0 and day 1 post infection also indicate the genomes likely represent virus particles that were non-infectious. At day 2 post infection, genomes/μl supernatant was approximately 25 fold greater than at day 0 or 1. At day 2 post infection, capsid transcripts/μl supernatant was approximately 300 fold greater than at day 0 or 1. At day 3 post infection, genomes/μl supernatant was approximately 350 fold greater than at day 0 or 1. At day 3 post infection, capsid transcripts/μl supernatant was approximately 450 fold greater than at day 0 or 1. The data shown in Table 11 indicated parvovirus B19 virus particles produced by the CD36⁺ erythroid progenitors cells was infectious.

TABLE 11

| | Approximate viral | | CP transcripts | CP transcripts |
|---|---|---|---|---|
| Day of | genomes detected | | detected on | detected on |
| supernatant | in supernatants | | Day 0 | Day 3 |
| harvest | (ge/mL) | MOI | (copies/mL) | (copies/mL) |
| Day 0 | 391 | 2 | ND | ND |
| Day 1 | 373 | 2 | ND | 9 |
| Day 2 | 10,500 | 52 | ND | 2,953 |
| Day 3 | 135,500 | 678 | 10 | 4,498 |

*ND means “not detected”

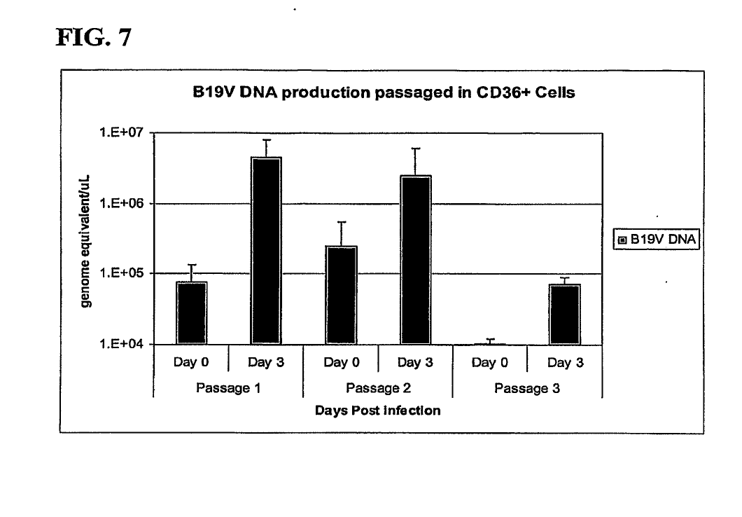

To demonstrate that the viral B19 DNA generated by infections with viremic plasma produced infectious particles, lysates of B19 infected CD36+ cells were used in two rounds of sequential infections. Infected cell lysates were freeze-thawed three times and clarified by centrifugation and applied directly or in serial dilutions to naïve cells. Cells were shown to produce 1-2 logs more infectious virus in two successive rounds of infection with lysates from infected cells. (FIG. 7).

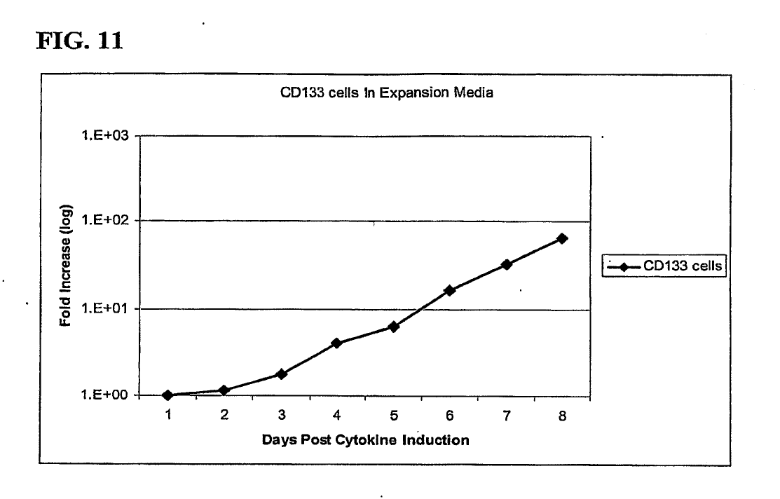

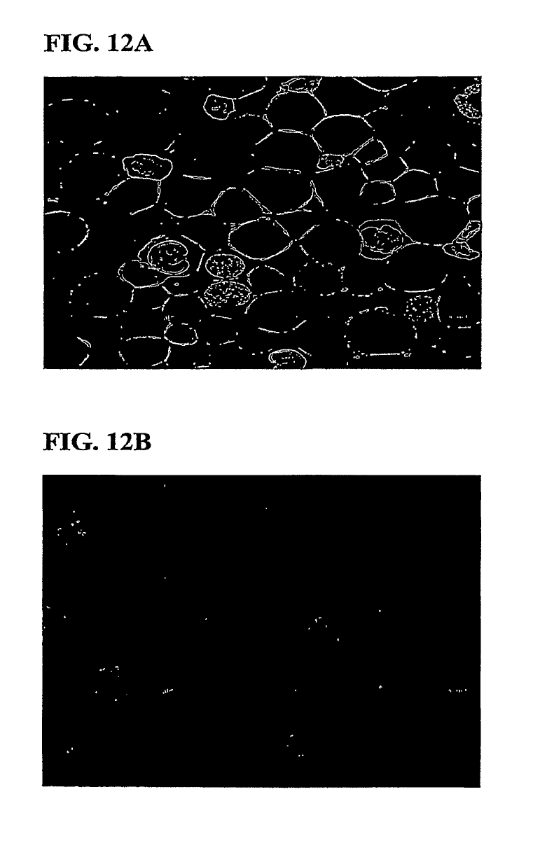

Expansion of CD133+ cells behave similarly to CD34+ cells. CD133⁺ cells culture in expansion media proliferate at a rate comparable to CD34⁺, increasing >1.8 logs within 8 days of culture (FIG. 11). Cells are also equally sensitive to B19 infection as seen in the immunofluorescence assay using the same assay for immunofluorescence with murine anti-B19 capsid protein monoclonal antibody 521-5D as the primary antibody and fluorescein isothiocyanate (FITC)-labeled goat anti-mouse IgG & IgM antibody as the secondary antibody. (FIG. 12A). The erythroid cells derived from CD133+ cells are CD36+, and have globoside. In appearance, the cells are similar to those derived from CD34+.

This methodology of production of CD36⁺ cells offers a better cellular system for in vitro infection assays with Parvovirus B19 as these cells are true erythroid progenitors. Moreover it is a flexible method, since it is adaptable to CD34⁺ cells, Cd133+, or other hematopoeitic stem cells obtained from different sources such as bone marrow, PBMC, PBSC, or cord blood. The CD36+ cells derived from this culture system were able to support viral infection and replication to a much higher degree than UT7/Epo-S1 cells, having a greater sensitivity of 2 logs detecting inoculations at 10e3 ge/mL and >3 log increase in viral DNA production.

When we looked for the capsid proteins by IF, we obtained 3 logs more sensitivity at 10e6 ge/mL in the CD36⁺ cells in comparison to the UT7/Epo-S1. Cells continue to be permissive to B19 infections at least up to D15 allowing for flexibility to work with these cells. In successive rounds of infections with lysates from infected cells, we can show that this system does produce infectious virus. We observed 1-2 log increases in viral DNA production in successive rounds of infection. B19 has been known to generate approximately 1 infectious unit in 10e3 to 10e5 genomes detected (Bonvicini, Gallinella et al., 2004), this may explain the decrease of viral DNA output among the second and third round infections compared to the initial round of infection.

With optimal transfection conditions, CD36⁺ cells transfected with the infectious clone pB19-M20 produced detectable infectious virus. This offers another potential source of infectious B19 virus and removes the dependency on viremic serum as an initial source of virus. Until now, the most reliable source of large amounts of B19 virus was phlebotomy of viremic donors and methods for consistently producing infectious B19 in a significant quantity in cell culture have been limited. Now with the ability to generate large scale numbers of cells highly permissive to B19 infection and a highly productive infection, we have cells capable of producing useful amounts of B19. Infectious virus is useful for identifying and developing therapeutically effective compositions for treatment and/or prevention of human parvovirus B19 infections, such as for example, antibodies, attenuated vaccines, and chimeric viral capsid proteins comprising antigenic epitopes.

### Example 2

### Transfection of CD36⁺Erythroid Progenitor Cells with an Infectious Parvovirus B19 Clone and Detection of Replicative Forms of Parvovirus B19 in the Transfected Cells

To determine if B19 could replicate in CD36⁺ erythroid progenitor cells, we used RT-PCR or qRT-PCR to detect transcripts for viral capsids in RNA recovered from transfected cells. The presence or absence of B19 capsid proteins was detected via immunofluorescent microscopy. By these experimental methods, the presence, transcription, and expression of the capsid gene could be confirmed.

### Methods

The conditions and reagents for transfecting plasmid DNA into CD36⁺ erythroid progenitor cells were first optimized using the plasmid pEGFP-F (BD Biosciences, Palo Alto, Calif.) that encodes farnesylated enhanced green fluorescent protein (EGFP). Cells were examined at daily intervals for expression of EGFP by UV microscopy and by FACS analysis. Conditions that gave the maximum number of cells expressing EGFP with minimum cytotoxicity were chosen. Such conditions are shown in Table 12 and are commercially available.

TABLE 12

| | | Nucleofector | |
|---|---|---|---|
| Cells | DNA | Reagent | Program |
| UT7/Epo-S1 | pEGFP/pB19-M20 | R | T-20 |
| CD36⁺ | pEGFP/pB19-M20 | R | T-20 |
| CD36⁺ | pEGFP/pB19-M20 | R | V-001 |
| CD36⁺ | pEGFP/pB19-M20 | V | T-19 |
| CD36⁺ | pEGFP/pB19-M20 | CD34 Prog. Cells | U-08 |
| CD36⁺ | pEGFP/pB19-M20 | Monocytic Cells | Y-001 |

For subsequent experiments, CD36⁺ erythroid progenitor cells were transfected after 8 days of culture in expansion medium using the AMAXA® Cell Line Nucleofector™ reagent V and program T19 according to the manufacturer's instructions (AMAXA Biosystems Inc., Nattermannallee, Germany). UT7/EPO-S1 cells were transfected using the AMAXA® Cell Line Nucleofector™ (reagent R and program T20 according to the manufacturer's instructions (Zhi et al., 2004, Virology, 318:142-152).

After day 8 in expansion media, 2×10⁶ CD36⁺ erythroid progenitor cells were transfected with 2 pg of plasmid pB19-M20 cut with SalI enzyme, which releases the full-length B19 genome from the plasmid (Zhi et al., 2004, Virology, 318:142-152). The percentage of mortality is higher (two times) than that observed in the UT7/Epo-S1 perhaps because the CD36⁺ cells are a primary culture type and not a cell line. Both the viability and the extent of positivity of the expression of GFP depend on the day of culture in which the transfection was performed. When the CD36⁺ cells are transfected at day 8 in expansion medium (confluence at 3×10⁵/ml), between 14% and 26% are positive for EGFP (depending on the condition used). When the cells are transfected at day 13 in expansion medium, we observed that the largest number of positive cells is with the monocyte kit, in contrast to day 8 and 10. Only 9% of CD36⁺ cells transfected at day 14 in expansion media is positive at the expression of GFP.

The CD36⁺ cells transfected with the infectious clone pB19-M20 with the different conditions were tested by IF after 48 hours post transfection. The best result was achieved with the condition Reagent V and Program T19, in which up to 50% of cells were positive by IF using antibody (521-5D) to the B19 capsid protein, where the Reagent R and Program T20 show a 40% of positivity and reagent for CD34 Progenitors and Monocytic cells around 10%. In comparing, transfection of CD36+ cells to UT7/Epo-S1 cells, the number of positive cells by IF after transfection with pB19-M20 is 10 times more in Cd36+ cells than observed with the UT7/Epo-S1 cells.

The cells were incubated for 72 hours post transfection, and then washed free of inoculum using fresh culture medium, and cell lysates prepared by three cycles of freeze/thawing. After centrifugation at 10,000 g for 10 min, the clarified supernatant was treated with RNase (final concentration of 1 U/μl, Roche Applied Science, Indianapolis, Ind.) and collected for further infections. The UT7/Epo-S1 cells were transfected with plasmid pB19-M20 as described in Zhi et al., 2004, Virology, 318:142-152.

Total RNA was extracted from the CD36⁺ erythroid progenitor cells UT7/Epo-S1 cells using RNA STAT-60™ (Tel-Test Inc., Friendswood, Tex.). Residual DNA was removed by DNAse I treatment (final concentration, 90 U/ml) for 15 min at room temperature. RNA was converted to cDNA with random primers and SuperScript™ II (Invitrogen), and RT-PCR for the spliced capsid transcripts was performed with primers B19-1 (5′GTTTTTTGTGAGCTAACTA3′; SEQ ID NO:321) and B19-9 (5′CCACGATGCAAGCTACAACTT3′; SEQ ID NO:322) as described in Nguyen et al., 2002, Virology, 301:374-380.

If required, mRNA was extracted from cells using a mRNA capture method (Qiagen Turbocapture) and directly reverse transcribed using M-MLV reverse transcriptase.

Transfected cells were cytocentrifuged (1500 rpm for 8 min in a Shandon cytospin 4 cytocentrifuge). The cells were fixed in acetone:methanol (1:1) at −20° C. for 5 min, washed twice in phosphate buffered saline (PBS) containing 0.1% fetal bovine serum, and incubated with a murine anti-B19 capsid protein monoclonal antibody (521-5D, gift of Larry Anderson, CDC) in PBS with 10% fetal calf serum for 1 hr at 37° C. After washing the slides twice in PBS, the slides were incubated with fluorescein isothiocyanate (FITC)-labeled goat anti-mouse IgG antibody (Jackson ImmunoResearch Laboratories, Inc., West Grove, Pa.) in PBS with 10% fetal calf serum and counterstained with Evans Blue for 30 mins at 37° C., washed in PBS, and examined by UV microscopy.

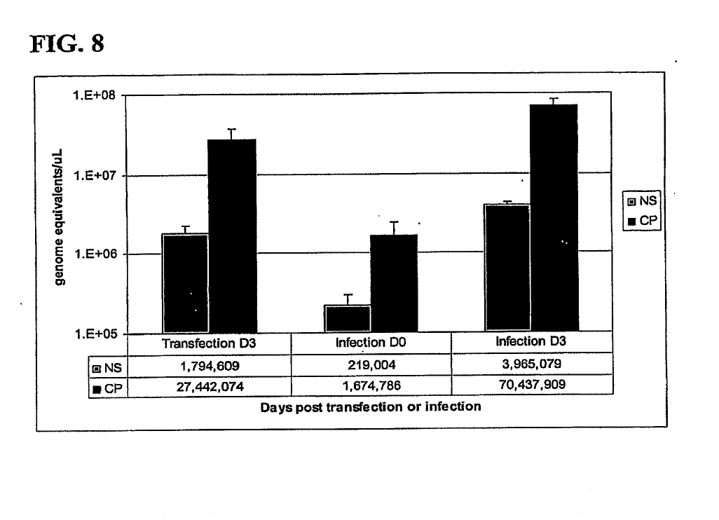

CD36⁺ day 8 cells proved to be the optimal day of expansion for cells to be transfected with pB19-M20 using the Nucleofection Amaxa System Reagent V and Program T19. Lysates from cells transfected with 2 μg of insert DNA, corresponding to the full-length genome of B19, or 5 μg of the whole plasmid pB19-M20 were used to infect naïve cells. For comparison, the same experiment was carried out using UT7/Epo-S1, following its optimized protocol (Reagent R and program T-20) for transfection with pB19-M20. CD36⁺ cells were infected with virus from viremic plasma as a positive control. As mentioned above, the transfection efficiency of pB19-M20 is much higher for CD36⁺ cells in comparison to UT7/Epo-S1 cells. Moreover, if we infect CD36⁺ cells at day 8 with the cell lysate from CD36⁺ transfected cells and assayed by qRT-PCR, infectious progeny efficiently infect naïve cells, detecting a >1.5 log increase in transcript production (FIG. 8). Using our RT-PCR method (Nguyen, Wong et al., 2002), viral mature RNA transcripts from infected cells are amplified as two alternatively spliced PCR products which are separated by gel electrophoresis and confirmed by southern hybridization analysis. RNA transcripts can be detected in transfected CD36⁺ cells and also were detected at day 3 post infection. We observed a number between 5 and 45 positive cells in IF, while we are not able to see positive cells in UT7/Epo-S1. (FIG. 12B).

### Results

The plasmid pEGFP-F was used to optimize the conditions for transfecting CD36⁺ erythroid progenitor cells. Although standard electroporation and liposomes were also tried, the best results were obtained using the AMAXA® Cell Line Nucleofector System™. The highest transfection efficiency (50%) with minimum cytotoxicity was achieved with reagent V and program T19 using 2 μg pEGFP DNA and 2×10⁶ CD36⁺ erythroid progenitor cells, following the manufacturer's instructions (AMAXA Biosystems Inc., Cologne, Germany).

CD36⁺ erythroid progenitor cells were transfected with plasmid pB19-M20 under the same conditions, and harvested at 72 h post-transfection. The RT-PCR and immunofluorescence assay were performed to detect viral spliced transcripts for capsid proteins or capsid proteins, respectively. After RT-PCR, two amplicons of 253 bp and 133 bp, representing the alternative spliced transcripts of B19 capsid gene, were detected in the cells transfected with pB19-M20 (data not shown). By immunofluorescence assay, B19 capsid protein was also detected in the transfected CD36⁺ erythroid progenitor cells, with approximately 50% of the cells having a positive signal when transfected with pB19-M20. The number CD36⁺ erythroid progenitor cells positive for B19 capsid protein was approximately 10 times greater than the number of UT7/Epo-S1 cells positive for the capsid protein. A greater than 1.5 log increase in infectious virus production was observed following transfection of CD36⁺ erythroid progenitor cells compared to transfection of UT7/Epo-S1 cells.

### Example 3

### Transfected CD36⁺Erythroid Progenitor Cells Produce B19 Infectious Virus

To determine if infectious virus were generated from the CD36⁺ erythroid progenitor cells transfected with pB19-M20, the cells were tested for B19 capsid expression by immunofluorescence and RNA extracted from cell lysates was tested for the transcripts of viral capsid or NS proteins by RT-PCR or qRT-PCR.

### Methods

For infection studies, 2×10⁴ of CD36⁺ erythroid progenitor cells in 10 μl of expansion medium were mixed with an equal volume of sample or positive control viral stock (J35 serum diluted to contain 10⁸ B19 genome copies) and incubated at 4° C. for 2 h to allow for maximum virus-cell interaction. The cells were then diluted to 2×10⁴ cells/0.1 ml or scaled up proportionately in the culture medium, and incubated at 37° C., in 5% CO₂. At 0-5 days post infection, cells were tested for evidence of infection by detection of viral transcripts and protein expression. To determine if infectious virus were generated from the CD36⁺ erythroid progenitor cells or UT7/Epo-S1 cells transfected with pB19-M20, the cells were assayed for B19 capsid expression by immunofluorescence as described above and the cell lysates was tested for the transcripts of viral capsid or NS protein genes by RT-PCR or qRT-pCR as described above. B19 infected CD36⁺ erythroid progenitor cells and UT7/Epo-S1 cells were used as a positive control.

### Results

The infected cultures were examined for the production of parvovirus B19 capsid proteins. At 72 h post-inoculation, capsid proteins could be detected in the nuclei and cytoplasm of cells with the supernatants derived from either B19 infection or pB19-M20 transfection.

### Example 4

### Transformation of CD36⁺ Erythroid Progenitor Cells with SV40 Large-T Antigen

The CD36⁺ erythroid progenitor cells in culture have a life span of about 20-23 days. In order to provide a consistent source of the CD36⁺ erythroid progenitor cells, we infected the cells with a viral vector encoding SV40 large-T antigen to extend the life span and replicative capacity of the CD36⁺ erythroid progenitor cell. Other viral vectors have also be used including: Lentivirus containing SV40 T-antigen; Lentivirus containing SV40 T-antigen plus a lentivirus containing hTERT (human telomerase reverse transcriptase gene); infection with EBV (Epstein-Barr virus); and a lentiviral vector containing the human papilloma virus (HPV) type 16 E6/E7 gene. Numerous plasmid and viral vectors are available commercially.

### Methods

The CD36⁺ erythroid progenitor cells were produced from CD34⁺ hematopoietic stem cells and cultured as described in Example 1. At day 8 of culture in the expansion media, 1.4×10⁷ cells were infected with 100 μl of recombinant adenovirus-SV40 (approximately 3×10⁸ PFU/ml; Gluzman et al., 1980, Proc. Natl. Acad. Sci. U.S.A., 77:3898-3902). The cells were incubated for 1 hr at 34° C., washed with expansion media, and resuspended in 10 ml expansion media. DNA and RNA analysis was performed as described in Example 1. Immunofluorescence and FACs analysis was performed as described in Example 1.

### Results

Similar to the culture of the CD36⁺ erythroid progenitor cells, the adenoviral-SV40 transformed CD36⁺ erythroid progenitor cells were non-enucleated and in some cases, visibly red, indicating the presence of hemoglobin. The CD36⁺ erythroid progenitor cells in culture had a life span of about 21 to 26 days. In contrast, the adenoviral-SV40 transformed CD36⁺ erythroid progenitor cells had a life span of about 25 to 30 days. FACs analysis of the transformed cells indicated that 1.23% of the cells were positive for CD34 and 99% of the cells were positive for CD36. CD19, CD3, and CD2 are cell surface markers for lymphocytes cells and can be used to distinguish erythroid progenitor cells from lymphoid lineage cells. CD44 is a cell surface marker for leukocytes and erythrocytes. FACs analysis indicated the transformed cells were CD44⁺, CD19⁻, CD10⁻, CD4⁻, CD3⁻, and CDT. A comparison of the surface antigens on the adenoviral-SV40 transformed CD36⁺ erythroid progenitor cells, CD34⁺ cells, CD36⁻ erythroid progenitor cells, and UT7/Epo-S1 cells is shown in Table 13.

TABLE 13

| | Cell Population (Percentage of cells | | | |
|---|---|---|---|---|
| | positive for surface marker) | | | |
| Surface | | | Transformed | |
| Antigen | CD34⁺ | CD36⁺ | CD36⁺ | UT7/Epo-S1 |
| Glycophorin A | 0.3 | 63.9 | NT | 26.6 |
| CD10 | 0.0 | 0.0 | 0.0 | 0.0 |
| CD19 | 0.1 | 0.0 | 0.0 | 0.0 |
| CD2 | 0.9 | 0.0 | 0.0 | 0.0 |
| CD3 | 0.1 | 0.0 | 0.0 | 0.0 |
| CD33 | 46.2 | 58.6 | 29.6 | 91.3 |
| CD34 | 96.6 | 1.0 | 0.0 | 0.0 |
| CD36 | 11.1 | 97.9 | 98.1 | 99.4 |
| CD44 | 98.9 | 98.0 | 98.2 | 99.7 |

*NT “means not tested”

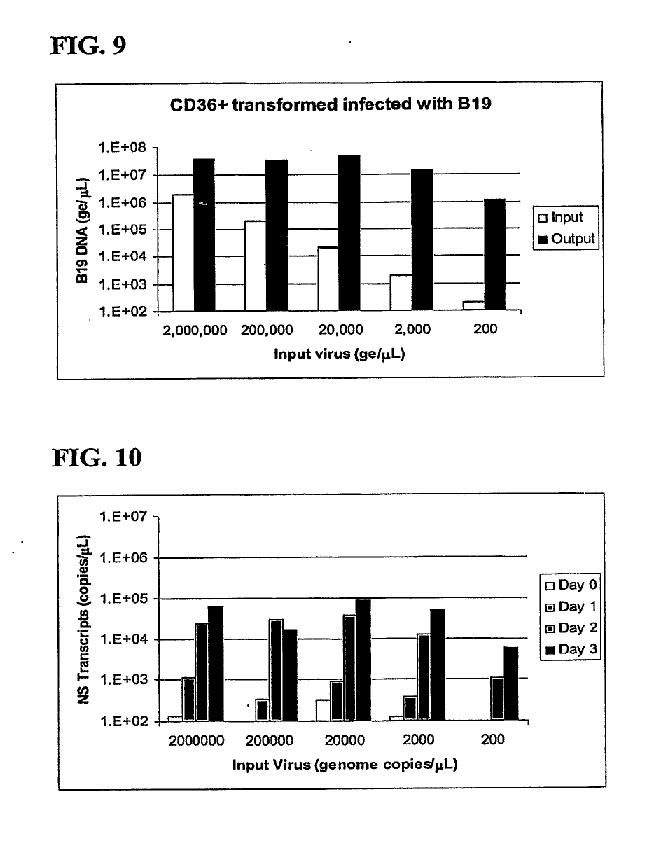

On Day 22 of culture in the expansion media (14 days post-transformation), the transformed CD36⁺ erythroid progenitor cells were infected with B19 as described in Example 1. The increase in B19 DNA was assayed by qPCR 3 days post infection. The transformed CD36⁺ cells are more sensitive to B19 infection than the non-transformed CD36⁺ cells (FIG. 9). Maximal virus output was observed with an input of 20,000 genomes/μl (ge/μl). An input of 200,000 ge/μl or 2,000,000 ge/μl into non-transformed CD36⁺ erythroid progenitors produced about the same output of virus observed for an input of 20,000 ge/μl.

B19 capsid protein in CD34⁺ cells, primary CD36⁻ cells, adenovirus-SV40 transformed CD36⁺ cells, CD36⁺ K562 cells and UT7/Epo-S1 cells was assayed by immunofluoresence 3 days post infection with B19. As shown in Table 14, at an multiplicity of Infection (MOI-ratio of virus to cells) of 10,000, the percent of adenovirus-SV40 transformed CD36⁺ erythroid progenitor cells positive for B19 capsid protein is approximately 2.5 fold greater than non-transformed CD36⁺ cells and approximately 23 fold greater than UT7/Epo-S1 cells.

TABLE 14

| | CD34* cells | Non- | Ad-SV40 | | |
|---|---|---|---|---|---|
| | (before | transformed | Transformed | | |
| MOI | differentiation) | CD36⁺ cells | CD36⁺ cells | K562/CD36+ | UT7/Epo-S1 |
| 100,000 | Neg | 14% | NT | NT | 4% |
| 10,000 | Neg | 9% | 23% | Neg | 1% |
| 1,000 | Neg | Pos | 15% | NT | Pos/neg |
| 100 | NT | Pos | 4% | NT | Pos/neg |
| 10 | NT | Pos | ~2% | NT | NT |
| 1 | NT | Pos | <1% | NT | NT |
| 0.1 | NT | Neg | <1% | NT | NT |
| 0.01 | NT | Neg | <1% | NT | NT |

*NT means not tested

NS transcripts from transfected CD36⁺ cells infected with B19 were quantitated by qRT-PCR. As shown in FIG. 10, CD36+ cells were infected with B19 and an increase in B19 NS transcripts was detected in all concentrations of input virus, in particular, a 2-3 log increase at some concentrations. The maximum number of B19 NS transcripts was observed 3 days post-infection with 10,000 genome copies/μl of virus.

### Example 5

### Microarray Expression Data

Using microarray technology, we have conducted time course studies which follow viral infection in CD36⁺ cells. We have also studied changes in gene expression as cells differentiate to permissivity for B19 infection.

Methods

CD36+ cells at a concentration of 2×10e5 cells/ml were infected with 10e9 B19 ge/mL. At various timepoints, cells were collected and RNA extracted using the Qiagen RNeasy micro kit. Hybridization cocktails for microarray analysis in Affymetrix GeneChips were produced following AffyMetrix's protocols.

Results

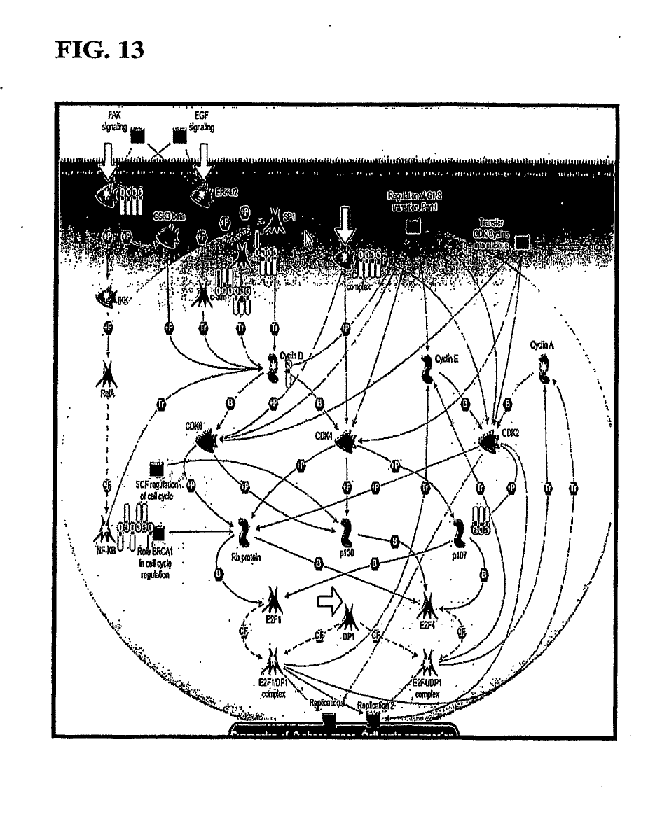

In this study, CD36+ cells were infected with B19 parvovirus and the samples collected at 0, 3, 6, 12, 24 and 48 h. The change in host cell gene expression induced by B19 infection was analyzed using Affymetrix GeneChip human arrays. A total of 7361 Genes were differentially (5-fold up or down) expressed during the progression of B19 infection (data not shown). We analyzed a total of 309 genes that were differentially expressed (more than 2-fold up or down and statistically significant using a FDR of 0.05) during B19 infection. In the early phase of infection (0, 3 and 6 h) a majority of differentially expressed genes were upregulated, and the genes were mainly involved in cytoskeleton remodeling, chemokines and/or adhesion molecules. In particular, the expression level of actin together with several proteins (alpha-actin, ACTR3/2, filamin A, and talin) associated with actin filaments were upregulated during the early phase of B19 infection. In addition, several critical factors (calmodulin, IP3R, PKC, and PKA) in calcium signaling also were targeted. In contrast, most differentially regulated genes declined during late phase infection (24 and 48 h) and were involved in growth arrest, cell metabolism, immune response, and apoptosis. The expression level of genes in the cyclosome/anaphase-promoting complex, a multisubunit E3 ubiquitin ligase targeting cell-cycle-related proteins, were significantly down regulated during the late phase of infection. Our data indicate that parvovirus B19 primarily targets cellular genes involved in cell architecture, cell-cycle regulation (FIG. 13), and calcium signaling.

The results in Table 15 show differential gene expression of genes expressed in B19 infected CD36+ cells. The genes shown had about a 2-fold difference from the expression at timepoint 0 (control). The change in expression level is shown at different time points post infection. The column showing upregulation or down regulation was determined by comparing expression levels at 48 hours compared to the 0 timepoint.

| | | | Hours Post-Infection | | | | | | 6 hr | 48 hr | SEQ ID |
|---|---|---|---|---|---|---|---|---|---|---|---|
| Probe set | Gene | GI # | 0 | 3 | 6 | 12 | 24 | 48 | Change | Change | NO |
| 213975_s_at | | 10731210 | 12.77 | 6.68 | 1.76 | 3.13 | 1.57 | 5.34 | down | down | 1 |
| 206207_at | LGALS10; | 20357558 | 2.84 | 2.64 | 1.41 | 2.35 | 1.46 | 4.35 | down | up | 2 |
| | MGC149659; | | | | | | | | | | |
| | LPPL_HUMAN | | | | | | | | | | |
| 219992_at | NKB; NKNB; | 55775470 | 1.04 | 0.89 | 1.01 | 0.74 | 0.96 | 3.92 | down | up | 3 |
| | PRO1155; | | | | | | | | | | |
| | ZNEUROK1 | | | | | | | | | | |
| 202437_s_at | NKB; NKNB; | 13325059 | 0.78 | 0.88 | 1.45 | 1.12 | 1.58 | 3.83 | up | up | 4 |
| | PRO1155; | | | | | | | | | | |
| | ZNEUROK1 | | | | | | | | | | |
| 206871_at | NE; HLE; HNE; | 58530849 | 8.71 | 5.49 | 1.83 | 2.92 | 1.34 | 3.64 | down | down | 5 |
| | PMN-E | | | | | | | | | | |
| 203949_at | IL6 | 4557758 | 6.12 | 4.00 | 1.79 | 2.82 | 1.55 | 3.21 | down | down | 6 |
| 203948_s_at | IL6 | 189039 | 6.74 | 3.76 | 1.56 | 2.51 | 1.15 | 3.12 | down | down | 7 |
| 202436_s_at | CYP1B1 | 11006376 | 1.23 | 0.83 | 1.66 | 1.29 | 1.57 | 3.12 | up | up | 8 |
| 205624_at | CDH13 | 4503000 | 2.99 | 2.39 | 1.33 | 1.68 | 1.17 | 2.95 | down | down | 9 |
| 200974_at | ACTSA | 4501882 | 1.16 | 1.12 | 1.10 | 1.09 | 1.71 | 2.94 | down | up | 10 |
| 200771_at | LAMB2; | 145309325 | 1.34 | 1.10 | 1.42 | 0.99 | 1.66 | 2.74 | up | up | 11 |
| | MGC87297 | | | | | | | | | | |
| AFFX- | | AFFX- | 1.22 | 1.01 | 1.10 | 1.01 | 1.09 | 2.71 | down | up | |
| M27830_M_at | | M27830_M | | | | | | | | | |
| 211734_s_at | FCE1A; | 13543505 | 3.40 | 2.14 | 1.30 | 2.05 | 1.30 | 2.71 | down | down | 12 |
| | FcERI | | | | | | | | | | |
| 201426_s_at | VIM | 5658563 | 3.11 | 2.30 | 0.92 | 1.82 | 0.99 | 2.70 | down | down | 13 |
| 202435_s_at | | 11016025 | 1.00 | 0.91 | 1.31 | 1.30 | 1.34 | 2.58 | up | up | 14 |
| AFFX- | | AFFX-M27830_5 | 0.83 | 1.06 | 1.27 | 1.09 | 1.05 | 2.55 | up | up | |
| M27830_5_at | | | | | | | | | | | |
| 219892_at | CCNC | 141803413 | 1.19 | 1.32 | 1.16 | 0.97 | 1.45 | 2.48 | down | up | 15 |
| 211005_at | LAT1; | 2828025 | 2.54 | 1.16 | 1.18 | 1.50 | 1.16 | 2.34 | down | down | 16 |
| | pp36 | | | | | | | | | | |
| 202662_s_at | IP3R2 | 95147334 | 1.04 | 1.17 | 1.02 | 1.24 | 1.39 | 2.25 | down | up | 17 |
| 205942_s_at | SA; SAH | 47458816 | 0.99 | 1.13 | 0.98 | 1.22 | 1.11 | 2.18 | down | up | 18 |
| 209795_at | CLEC2C | 291897 | 3.83 | 1.01 | 1.31 | 1.09 | 1.38 | 2.11 | down | down | 19 |
| 218788_s_at | ZMYND1; | 12232400 | 0.93 | 1.13 | 1.10 | 1.11 | 1.37 | 2.08 | up | up | 20 |
| | ZNFN3A1; | | | | | | | | | | |
| | FLJ21080; | | | | | | | | | | |
| | MGC104324; | | | | | | | | | | |
| | bA74P14.1 | | | | | | | | | | |
| 204802_at | RAD; RAD1; | 4759053 | 0.67 | 1.00 | 1.00 | 0.97 | 1.25 | 2.07 | up | up | 21 |
| | REM3 | | | | | | | | | | |
| 214575_s_at | AZU; HBP; | 28416954 | 5.04 | 3.50 | 1.38 | 2.34 | 1.25 | 2.05 | down | down | 22 |
| | NAZC; AZAMP; | | | | | | | | | | |
| | CAP37; | | | | | | | | | | |
| | HUMAZUR | | | | | | | | | | |
| 207813_s_at | ADXR | 111118982 | 0.85 | 1.00 | 0.86 | 1.07 | 1.23 | 2.00 | up | up | 23 |
| 207957_s_at | PKCB; PRKCB; | 47157320 | 2.12 | 1.05 | 0.97 | 1.23 | 1.37 | 2.00 | down | down | 24 |
| | PRKCB2; | | | | | | | | | | |
| | MGC41878; | | | | | | | | | | |
| | PKC-beta | | | | | | | | | | |
| 205780_at | BP4; | 21536418 | 0.94 | 0.83 | 1.16 | 0.95 | 1.26 | 1.93 | up | up | 25 |
| | NBK; | | | | | | | | | | |
| | BIP1 | | | | | | | | | | |
| 210886_x_at | P53TG1; | 5006270 | 0.79 | 1.11 | 0.99 | 1.20 | 1.27 | 1.93 | up | up | 26 |
| | TP53TG1; | | | | | | | | | | |
| | P53TG1-D; | | | | | | | | | | |
| | H_RG012D21.9; | | | | | | | | | | |
| | TP53 target | | | | | | | | | | |
| | gene 1 | | | | | | | | | | |
| 219763_at | FAM31A; | 55749788 | 0.82 | 1.01 | 1.20 | 1.02 | 1.49 | 1.93 | up | up | 27 |
| | FLJ38464; | | | | | | | | | | |
| | KIAA1608; | | | | | | | | | | |
| | RP11-230L22.3 | | | | | | | | | | |
| 212099_at | GRB2 | 3872112 | 2.40 | 0.80 | 1.29 | 1.24 | 0.93 | 1.92 | down | down | 28 |
| 202890_at | MAP7 | 6576051 | 0.94 | 1.19 | 1.01 | 1.07 | 1.29 | 1.91 | up | up | 29 |
| 205229_s_at | COCH | 2630835 | 0.56 | 0.87 | 1.22 | 1.02 | 1.07 | 1.90 | up | up | 30 |
| 213107_at | | 829788 | 0.92 | 0.88 | 1.33 | 0.84 | 1.14 | 1.86 | up | up | 31 |
| 203471_s_at | P47; | 4505878 | 2.72 | 1.38 | 1.22 | 1.36 | 1.14 | 1.86 | down | down | 32 |
| | FLJ27168 | | | | | | | | | | |
| 202766_s_at | FBN; SGS; | 93589095 | 0.65 | 0.80 | 1.21 | 0.97 | 1.48 | 1.86 | up | up | 33 |
| | WMS; MASS; | | | | | | | | | | |
| | MFS1; OCTD | | | | | | | | | | |
| 203381_s_at | TOMM40 | 1153408 | 3.37 | 1.71 | 0.96 | 1.40 | 0.98 | 1.85 | down | down | 34 |
| 209881_s_at | LAT1; | 2828023 | 2.73 | 1.46 | 1.11 | 1.42 | 1.24 | 1.84 | down | down | 35 |
| | pp36 | | | | | | | | | | |
| 208894_at | HLA- | 188255 | 3.25 | 2.81 | 1.14 | 1.79 | 1.04 | 1.81 | down | down | 36 |
| | DRA1 | | | | | | | | | | |
| AFFX-r2-Bs-lys- | | AFFX-r2-Bs-lys-3 | 0.85 | 1.09 | 1.09 | 0.88 | 0.88 | 1.81 | up | up | |
| 3_at | | | | | | | | | | | |
| 217763_s_at | Rab22B | 33589860 | 0.89 | 1.06 | 1.03 | 1.20 | 1.23 | 1.80 | up | up | 37 |
| 201631_s_at | DIF2; IEX1; | 119964722 | 2.23 | 1.15 | 1.58 | 1.15 | 0.97 | 1.80 | down | down | 38 |
| | PRG1; DIF-2; | | | | | | | | | | |
| | GLY96; IEX-1; | | | | | | | | | | |
| | IEX-1L | | | | | | | | | | |
| 209685_s_at | PKCB; PRKCB; | 189968 | 2.22 | 1.51 | 0.94 | 1.35 | 1.34 | 1.79 | down | down | 39 |
| | PRKCB2; | | | | | | | | | | |
| | MGC41878; | | | | | | | | | | |
| | PKC-beta | | | | | | | | | | |
| 215785_s_at | PIR121 | 7328000 | 2.10 | 1.04 | 1.13 | 1.13 | 1.21 | 1.76 | down | down | 40 |
| 200644_at | F52; MLP; | 32401423 | 0.72 | 0.92 | 1.02 | 1.00 | 1.28 | 1.75 | up | up | 41 |
| | MRP; MLP1; | | | | | | | | | | |
| | MACMARCKS | | | | | | | | | | |
| 204174_at | FLAP | 15718674 | 2.92 | 2.15 | 1.30 | 1.39 | 1.15 | 1.74 | down | down | 42 |
| 210254_at | HTM4; | 561638 | 2.85 | 2.01 | 1.35 | 2.37 | 1.38 | 1.74 | down | down | 43 |
| | CD20L | | | | | | | | | | |
| 207067_s_at | HDC | 92110054 | 2.83 | 2.26 | 1.28 | 1.52 | 1.10 | 1.73 | down | down | 44 |
| 212224_at | ALDC; ALDH1; | 25777722 | 0.70 | 0.98 | 1.15 | 1.06 | 1.13 | 1.71 | up | up | 45 |
| | PUMB1; | | | | | | | | | | |
| | ALDH11; | | | | | | | | | | |
| | RALDH1; | | | | | | | | | | |
| | ALDH-E1; | | | | | | | | | | |
| | MGC2318 | | | | | | | | | | |
| 213142_x_at | TERF2IP | 10302386 | 0.72 | 1.00 | 1.18 | 0.85 | 1.50 | 1.71 | up | up | 46 |
| 209083_at | p57; TACO; | 1002922 | 2.02 | 1.31 | 0.80 | 1.36 | 1.00 | 1.71 | down | down | 47 |
| | CLABP; | | | | | | | | | | |
| | HCORO1; | | | | | | | | | | |
| | CLIPINA; | | | | | | | | | | |
| | FLJ41407; | | | | | | | | | | |
| | MGC117380 | | | | | | | | | | |
| 209803_s_at | IPL; BRW1C; | 2150049 | 1.61 | 0.79 | 1.26 | 0.95 | 1.30 | 1.68 | down | up | 48 |
| | BWR1C; | | | | | | | | | | |
| | HLDA2; TSSC3 | | | | | | | | | | |
| 218711_s_at | SDR; PS- | 66346738 | 1.85 | 1.23 | 0.89 | 1.51 | 1.14 | 1.67 | down | down | 49 |
| | p68 | | | | | | | | | | |
| 204803_s_at | RAD; RAD1; | 4759053 | 0.59 | 0.77 | 1.22 | 0.97 | 0.99 | 1.66 | up | up | 50 |
| | REM3 | | | | | | | | | | |
| 206023_at | NMU | 5729946 | 0.60 | 0.65 | 1.13 | 0.88 | 1.16 | 1.64 | up | up | 51 |
| 218924_s_at | CTB | 4758091 | 0.77 | 1.11 | 1.18 | 0.92 | 1.06 | 1.63 | up | up | 52 |
| 34210_at | | 1444193 | 2.97 | 1.76 | 1.05 | 1.37 | 1.23 | 1.63 | down | down | 53 |
| 210609_s_at | PIG3 | 33875490 | 0.76 | 0.77 | 1.13 | 0.94 | 1.20 | 1.63 | up | up | 54 |
| 201850_at | MCP; | 63252912 | 4.01 | 2.21 | 1.34 | 1.94 | 1.17 | 1.61 | down | down | 55 |
| | AFCP | | | | | | | | | | |
| 214453_s_at | p44; | 141802167 | 0.96 | 1.07 | 1.60 | 2.12 | 1.48 | 1.60 | up | up | 56 |
| | MTAP44 | | | | | | | | | | |
| 210982_s_at | HLA- | 188268 | 2.47 | 2.73 | 1.02 | 1.43 | 0.97 | 1.59 | down | down | 57 |
| | DRA1 | | | | | | | | | | |
| 201373_at | HD1; PCN; | 47607491 | 1.69 | 1.09 | 0.82 | 1.20 | 1.05 | 1.58 | down | down | 58 |
| | EBS1; EBSO; | | | | | | | | | | |
| | PLTN; PLEC1b | | | | | | | | | | |
| 209312_x_at | DRB1; HLA | 5478215 | 2.28 | 1.59 | 1.32 | 1.59 | 1.09 | 1.58 | down | down | 59 |
| | DRB1; HLA- | | | | | | | | | | |
| | DR1B | | | | | | | | | | |
| 202071_at | SYND4; | 38201674 | 0.64 | 0.81 | 1.07 | 1.10 | 1.21 | 1.54 | up | up | 60 |
| | MGC22217 | | | | | | | | | | |
| 205683_x_at | TPS1; TPS2; | 61744442 | 2.26 | 1.45 | 1.03 | 1.22 | 1.14 | 1.53 | down | down | 61 |
| | TPSB1; alpha II | | | | | | | | | | |
| 219654_at | APOBEC3F | 82659104 | 0.74 | 1.07 | 1.03 | 1.09 | 1.12 | 1.53 | up | up | 62 |
| 204017_at | ERD2L3 | 8051612 | 0.78 | 0.99 | 1.35 | 1.18 | 1.57 | 1.50 | up | up | 63 |
| 202627_s_at | SERPINE1 | 31295545 | 3.46 | 1.48 | 1.04 | 1.24 | 0.92 | 1.50 | down | down | 64 |
| 207134_x_at | TPS2; TPSB1; | 68508969 | 2.10 | 1.33 | 1.04 | 1.20 | 1.18 | 1.50 | down | down | 65 |
| | tryptaseC | | | | | | | | | | |
| 217023_x_at | TPS2; TPSB1; | 4336616 | 2.35 | 1.41 | 0.93 | 1.42 | 1.16 | 1.48 | down | down | 66 |
| | tryptaseC | | | | | | | | | | |
| 205625_s_at | CALB1 | 5863684 | 0.35 | 0.73 | 1.13 | 0.80 | 1.12 | 1.48 | up | up | 67 |
| 202075_s_at | PLTP | 33356542 | 0.72 | 0.79 | 0.98 | 1.13 | 1.35 | 1.47 | up | up | 68 |
| 216920_s_at | TARP | 540458 | 2.06 | 2.10 | 1.06 | 1.40 | 0.95 | 1.46 | down | down | 69 |
| 216474_x_at | TPS1; TPS2; | 11493901 | 2.12 | 1.30 | 1.01 | 1.26 | 1.20 | 1.43 | down | down | 70 |
| | TPSB1; alpha II | | | | | | | | | | |
| 202069_s_at | PXDN | 5446731 | 1.82 | 1.45 | 0.90 | 1.22 | 1.29 | 1.41 | down | down | 71 |
| 201666_at | EPA; EPO; HCI; | 73858576 | 2.41 | 1.68 | 1.01 | 1.32 | 1.14 | 1.41 | down | down | 72 |
| | CLGI; TIMP; | | | | | | | | | | |
| | FLJ90373 | | | | | | | | | | |
| 209771_x_at | | 2810111 | 2.29 | 1.28 | 1.14 | 1.82 | 1.24 | 1.41 | down | down | 73 |
| 215382_x_at | TPS1; TPS2; | 11493899 | 1.98 | 1.35 | 0.97 | 1.20 | 1.15 | 1.41 | down | down | 74 |
| | TPSB1; alpha II | | | | | | | | | | |
| 209288_s_at | UB1; CEP3; | 6807668 | 2.88 | 1.39 | 1.33 | 1.18 | 1.23 | 1.40 | down | down | 75 |
| | BORG2; | | | | | | | | | | |
| | FLJ46903 | | | | | | | | | | |
| 205270_s_at | SLP76; | 47078282 | 2.24 | 1.48 | 0.99 | 1.47 | 1.34 | 1.40 | down | down | 76 |
| | SLP-76 | | | | | | | | | | |
| 206519_x_at | CD33L; | 2913994 | 2.24 | 0.96 | 1.12 | 1.30 | 1.04 | 1.39 | down | down | 77 |
| | OBBP1; | | | | | | | | | | |
| | CD33L1; | | | | | | | | | | |
| | CDw327; | | | | | | | | | | |
| | SIGLEC-6 | | | | | | | | | | |
| 201952_at | ALCAM | 1728335 | 2.12 | 1.50 | 1.19 | 1.20 | 0.95 | 1.39 | down | down | 78 |
| 214240_at | GAL | 46181832 | 0.48 | 0.61 | 0.91 | 0.92 | 1.08 | 1.38 | up | up | 79 |
| 204661_at | CDW52 | 68342029 | 2.62 | 1.53 | 0.94 | 1.31 | 1.18 | 1.37 | down | down | 80 |
| 211028_s_at | TRAP1 | 33873491 | 0.66 | 0.97 | 1.16 | 0.75 | 1.12 | 1.37 | up | up | 81 |
| 207741_x_at | TPSAB1 | 61744442 | 2.00 | 1.37 | 0.96 | 1.35 | 1.16 | 1.36 | down | down | 82 |
| 210084_x_at | TPS1; TPS2; | 11493897 | 2.04 | 1.37 | 0.95 | 1.37 | 1.17 | 1.35 | down | down | 83 |
| | TPSB1; alpha II | | | | | | | | | | |
| 202917_s_at | P8; MIF; NIF; | 21614543 | 2.33 | 1.31 | 1.20 | 1.55 | 1.14 | 1.35 | down | down | 84 |
| | CAGA; CFAG; | | | | | | | | | | |
| | CGLA; L1Ag; | | | | | | | | | | |
| | MRP8; CP-10; | | | | | | | | | | |
| | MA387; | | | | | | | | | | |
| | 60B8AG | | | | | | | | | | |
| 205692_s_at | T10 | 38454325 | 2.07 | 1.50 | 1.22 | 1.28 | 0.97 | 1.34 | down | down | 85 |
| 206446_s_at | ELA1 | 58331208 | 2.32 | 1.04 | 1.45 | 1.06 | 1.09 | 1.33 | down | down | 86 |
| 200660_at | MLN70; | 5032056 | 2.08 | 1.61 | 1.02 | 1.34 | 1.18 | 1.32 | down | down | 87 |
| | S100C | | | | | | | | | | |
| 203305_at | F13A | 119395708 | 1.88 | 0.77 | 1.37 | 0.90 | 1.24 | 1.32 | down | down | 88 |
| 205626_s_at | CALB | 5579451 | 0.42 | 0.51 | 1.16 | 1.20 | 1.12 | 1.31 | up | up | 89 |
| 202388_at | G0S8 | 142365756 | 2.97 | 1.26 | 1.20 | 0.88 | 0.93 | 1.31 | down | down | 90 |
| 202411_at | P27; ISG12; | 55925613 | 0.77 | 0.81 | 1.10 | 2.26 | 1.74 | 1.31 | up | up | 91 |
| | FAM14D | | | | | | | | | | |
| 211031_s_at | CLIP; CLIP2; | 33988839 | 1.84 | 1.21 | 0.91 | 1.15 | 0.95 | 1.31 | down | down | 92 |
| | WSCR4; | | | | | | | | | | |
| | WBSCR4; | | | | | | | | | | |
| | CLIP-115; | | | | | | | | | | |
| | KIAA0291; | | | | | | | | | | |
| | MGC11333 | | | | | | | | | | |
| 217028_at | FB22; HM89; | 3059119 | 2.24 | 2.10 | 1.35 | 1.20 | 1.09 | 1.29 | down | down | 93 |
| | LAP3; LCR1; | | | | | | | | | | |
| | NPYR; WHIM; | | | | | | | | | | |
| | CD184; LESTR; | | | | | | | | | | |
| | NPY3R; | | | | | | | | | | |
| | NPYRL; | | | | | | | | | | |
| | HSY3RR; | | | | | | | | | | |
| | NPYY3R; | | | | | | | | | | |
| | D2S201E | | | | | | | | | | |
| 203382_s_at | AD2; | 48762938 | 3.73 | 1.31 | 0.81 | 1.29 | 1.29 | 1.29 | down | down | 94 |
| | MGC1571; | | | | | | | | | | |
| | apoprotein | | | | | | | | | | |
| 204415_at | 6-16; G1P3; | 94538330 | 0.93 | 1.16 | 1.11 | 2.40 | 1.75 | 1.29 | up | up | 95 |
| | FAM14C; | | | | | | | | | | |
| | IFI616; IFI-6-16 | | | | | | | | | | |
| 217975_at | DKFZp313K1940 | 55925651 | 0.61 | 0.82 | 1.23 | 0.87 | 1.20 | 1.29 | up | up | 96 |
| 207341_at | MBT; P29; | 71361687 | 4.41 | 2.33 | 1.28 | 1.94 | 1.27 | 1.29 | down | down | 97 |
| | ACPA; AGP7; | | | | | | | | | | |
| | PR-3; C-ANCA | | | | | | | | | | |
| 200999_s_at | p63; CLIMP-63; | 19920316 | 2.31 | 1.47 | 1.19 | 1.48 | 1.10 | 1.28 | down | down | 98 |
| | ERGIC-63; | | | | | | | | | | |
| | MGC99554 | | | | | | | | | | |
| 205269_at | LCP2 | 3539017 | 2.48 | 0.82 | 1.13 | 1.39 | 1.48 | 1.27 | down | down | 99 |
| 212794_s_at | KIAA1033 | 7023169 | 2.18 | 0.87 | 1.10 | 0.96 | 1.16 | 1.27 | down | down | 100 |
| 203473_at | OATPB; OATP- | 41152057 | 2.32 | 1.16 | 0.97 | 1.13 | 1.12 | 1.26 | down | down | 101 |
| | B; OATP2B1; | | | | | | | | | | |
| | SLC21A9; | | | | | | | | | | |
| | KIAA0880; | | | | | | | | | | |
| | DKFZp686E0517 | | | | | | | | | | |
| 209014_at | NRAGE; | 9963809 | 0.61 | 0.85 | 1.12 | 1.04 | 1.05 | 1.26 | up | up | 102 |
| | DLXIN-1 | | | | | | | | | | |
| 210017_at | MALT1 | 3387883 | 0.58 | 1.02 | 1.04 | 0.89 | 1.26 | 1.24 | up | up | 103 |
| 222240_s_at | ISYNA1 | 6808384 | 0.45 | 0.70 | 0.80 | 1.06 | 1.11 | 1.24 | up | up | 104 |
| 215806_x_at | TCRGC2; | 339168 | 2.30 | 1.68 | 1.11 | 1.27 | 1.05 | 1.24 | down | down | 105 |
| | TRGC2(2X); | | | | | | | | | | |
| | TRGC2(3X); T- | | | | | | | | | | |
| | cell receptor, | | | | | | | | | | |
| | gamma, | | | | | | | | | | |
| | constant region | | | | | | | | | | |
| | C2 | | | | | | | | | | |
| 208309_s_at | MLT; MLT1; | 27886564 | 0.60 | 0.80 | 0.95 | 0.93 | 1.32 | 1.23 | up | up | 106 |
| | DKFZp434L132 | | | | | | | | | | |
| 205266_at | CDF; HILDA; D- | 6006018 | 2.28 | 1.31 | 1.13 | 1.24 | 0.92 | 1.23 | down | down | 107 |
| | FACTOR | | | | | | | | | | |
| 222303_at | ETS2 | 10302862 | 2.84 | 1.45 | 1.49 | 1.45 | 0.88 | 1.22 | down | down | 108 |
| 203518_at | CHS; | 54292122 | 1.72 | 1.58 | 1.12 | 0.82 | 0.91 | 1.21 | down | down | 109 |
| | CHS1 | | | | | | | | | | |
| 202597_at | POLR2J | 11005805 | 0.57 | 0.84 | 1.12 | 1.00 | 1.16 | 1.20 | up | up | 110 |
| 204103_at | ACT2; G-26; | 90704850 | 1.85 | 1.22 | 0.97 | 1.11 | 0.82 | 1.20 | down | down | 111 |
| | LAG1; MIP1B; | | | | | | | | | | |
| | SCYA2; | | | | | | | | | | |
| | SCYA4; | | | | | | | | | | |
| | AT744.1; | | | | | | | | | | |
| | MGC104418; | | | | | | | | | | |
| | MGC126025; | | | | | | | | | | |
| | MGC126026; | | | | | | | | | | |
| | MIP-1-beta | | | | | | | | | | |
| 201005_at | 5H9; BA2; P24; | 21237762 | 2.94 | 1.26 | 1.05 | 1.38 | 1.20 | 1.20 | down | down | 112 |
| | GIG2; MIC3; | | | | | | | | | | |
| | MRP-1; BTCC- | | | | | | | | | | |
| | 1; DRAP-27; | | | | | | | | | | |
| | TSPAN29 | | | | | | | | | | |
| 221944_at | FLJ42627 | 1200802 | 1.75 | 1.16 | 0.80 | 1.27 | 1.09 | 1.20 | down | down | 113 |
| 202859_x_at | K60; NAF; | 28610153 | 5.65 | 1.94 | 2.23 | 1.12 | 1.27 | 1.20 | down | down | 114 |
| | GCP1; IL-8; | | | | | | | | | | |
| | LECT; LUCT; | | | | | | | | | | |
| | NAP1; 3-10C; | | | | | | | | | | |
| | CXCL8; GCP-1; | | | | | | | | | | |
| | LYNAP; | | | | | | | | | | |
| | MDNCF; | | | | | | | | | | |
| | MONAP; NAP- | | | | | | | | | | |
| | 1; SCYB8; | | | | | | | | | | |
| | TSG-1; AMCF-I; | | | | | | | | | | |
| | b-ENAP | | | | | | | | | | |
| 213524_s_at | RP1- | 20070269 | 0.42 | 0.60 | 1.00 | 0.88 | 1.10 | 1.19 | up | up | 115 |
| | 28O10.2 | | | | | | | | | | |
| 201688_s_at | TPD52 | 13282461 | 0.78 | 1.04 | 0.74 | 1.06 | 1.64 | 1.19 | down | up | 116 |
| 202504_at | ATDC | 109826574 | 0.38 | 0.60 | 1.00 | 0.94 | 1.11 | 1.18 | up | up | 117 |
| 209201_x_at | FB22; HM89; | 189313 | 2.17 | 1.94 | 0.91 | 1.22 | 1.17 | 1.18 | down | down | 118 |
| | LAP3; LCR1; | | | | | | | | | | |
| | NPYR; WHIM; | | | | | | | | | | |
| | CD184; LESTR; | | | | | | | | | | |
| | NPY3R; | | | | | | | | | | |
| | NPYRL; | | | | | | | | | | |
| | HSY3RR; | | | | | | | | | | |
| | NPYY3R; | | | | | | | | | | |
| | D2S201E | | | | | | | | | | |
| 209806_at | H2B/S; H2BFT; | 12654150 | 0.50 | 0.67 | 1.03 | 0.70 | 0.74 | 1.18 | up | up | 119 |
| | H2BFAiii; | | | | | | | | | | |
| | MGC131989 | | | | | | | | | | |
| 202718_at | IBP2; IGF- | 55925575 | 4.03 | 1.65 | 1.14 | 1.41 | 1.18 | 1.18 | down | down | 120 |
| | BP53 | | | | | | | | | | |
| 203505_at | TGD; ABC1; | 9755158 | 0.18 | 0.41 | 0.98 | 1.12 | 0.76 | 1.17 | up | up | 121 |
| | CERP; ABC-1; | | | | | | | | | | |
| | HDLDT1; | | | | | | | | | | |
| | FLJ14958 | | | | | | | | | | |
| 205483_s_at | G1P2; UCRP; | 142368098 | 1.12 | 1.00 | 1.29 | 3.09 | 1.84 | 1.17 | up | up | 122 |
| | IFI15 | | | | | | | | | | |
| 211796_s_at | APC | 3002924 | 1.84 | 0.91 | 1.09 | 1.43 | 1.34 | 1.16 | down | down | 123 |
| 202731_at | H731; | 34304340 | 0.55 | 0.80 | 0.88 | 0.95 | 1.02 | 1.14 | up | up | 124 |
| | MGC33046; | | | | | | | | | | |
| | MGC33047 | | | | | | | | | | |
| 211002_s_at | ATDC | 12275865 | 0.62 | 0.56 | 0.93 | 1.07 | 1.27 | 1.14 | up | up | 125 |
| 203153_at | G10P1; IFI56; | 116534936 | 0.89 | 1.00 | 1.57 | 2.26 | 1.22 | 1.13 | up | up | 126 |
| | IFI-56; IFNAI1; | | | | | | | | | | |
| | RNM561; | | | | | | | | | | |
| | GARG-16 | | | | | | | | | | |
| 218988_at | BLOV1 | 56699410 | 0.58 | 1.01 | 1.38 | 0.90 | 1.01 | 1.12 | up | up | 127 |
| 214132_at | ATP5C1 | 12727189 | 0.73 | 0.92 | 1.61 | 0.92 | 0.85 | 1.11 | up | up | 128 |
| 203504_s_at | TGD; ABC1; | 21536375 | 0.29 | 0.79 | 1.04 | 0.98 | 0.93 | 1.10 | up | up | 129 |
| | CERP; ABC-1; | | | | | | | | | | |
| | HDLDT1; | | | | | | | | | | |
| | FLJ14958 | | | | | | | | | | |
| 219426_at | AGO3; | 29294646 | 1.83 | 1.26 | 1.09 | 0.87 | 1.25 | 1.10 | down | down | 130 |
| | FLJ12765; | | | | | | | | | | |
| | MGC86946 | | | | | | | | | | |
| 206693_at | IL-7 | 28610152 | 1.66 | 0.70 | 0.91 | 1.09 | 1.25 | 1.09 | down | down | 131 |
| 210018_x_at | MLT; MLT1; | 5706377 | 0.67 | 0.74 | 0.93 | 0.92 | 1.38 | 1.09 | up | up | 132 |
| | DKFZp434L132 | | | | | | | | | | |
| 206976_s_at | HSP105A; | 42544158 | 1.98 | 1.70 | 1.01 | 0.95 | 0.98 | 1.09 | down | down | 133 |
| | HSP105B; | | | | | | | | | | |
| | KIAA0201; NY- | | | | | | | | | | |
| | CO-25; | | | | | | | | | | |
| | DKFZp686M05240 | | | | | | | | | | |
| 215936_s_at | KIAA1033 | 7023048 | 1.90 | 1.23 | 0.93 | 1.18 | 0.99 | 1.09 | down | down | 134 |
| 218566_s_at | CHP1 | 6912303 | 2.52 | 1.48 | 1.00 | 1.00 | 1.13 | 1.08 | down | down | 135 |
| 201650_at | K19; CK19, | 131412244 | 0.37 | 0.65 | 0.99 | 0.78 | 1.02 | 1.08 | up | up | 136 |
| | K1CS; | | | | | | | | | | |
| | MGC15366 | | | | | | | | | | |
| 208744_x_at | HSPH1 | 13297108 | 2.35 | 1.65 | 0.72 | 0.99 | 1.09 | 1.07 | down | down | 137 |
| 209376_x_at | POLQ | 6039911 | 1.69 | 0.99 | 0.84 | 1.10 | 1.00 | 1.07 | down | down | 138 |
| 210358_x_at | NFE1B; | 33876862 | 1.91 | 0.92 | 0.96 | 1.13 | 1.20 | 1.06 | down | down | 139 |
| | MGC2306 | | | | | | | | | | |
| 207076_s_at | ASS; | 113204625 | 2.06 | 1.29 | 1.26 | 0.90 | 1.10 | 1.05 | down | down | 140 |
| | CTLN1 | | | | | | | | | | |
| 204232_at | FCER1G | 4758343 | 2.15 | 1.14 | 1.01 | 1.32 | 1.07 | 1.04 | down | down | 141 |
| 216392_s_at | P125; | 10433116 | 1.65 | 1.54 | 0.78 | 0.96 | 1.08 | 1.04 | down | down | 142 |
| | MSTP053 | | | | | | | | | | |
| 214291_at | RPL17 | 2263330 | 1.67 | 0.81 | 1.11 | 0.96 | 0.90 | 1.03 | down | down | 143 |
| 212105_s_at | DHX9 | 11261901 | 1.37 | 0.93 | 0.44 | 0.89 | 1.00 | 1.03 | down | down | 144 |
| 205919_at | HBE1 | 28302129 | 1.45 | 0.83 | 0.75 | 1.08 | 0.70 | 1.01 | down | down | 145 |
| 206520_x_at | CD33L; | 87298825 | 1.84 | 0.86 | 1.11 | 1.10 | 1.20 | 1.01 | down | down | 146 |
| | OBBP1; | | | | | | | | | | |
| | CD33L1; | | | | | | | | | | |
| | CDw327; | | | | | | | | | | |
| | SIGLEC-6 | | | | | | | | | | |
| 209774_x_at | GRO2; GROb; | 183626 | 3.50 | 1.14 | 1.12 | 1.14 | 0.91 | 1.01 | down | down | 147 |
| | MIP2; MIP2A; | | | | | | | | | | |
| | SCYB2; MGSA- | | | | | | | | | | |
| | b; MIP-2a; | | | | | | | | | | |
| | CINC-2a; | | | | | | | | | | |
| | MGSA beta | | | | | | | | | | |
| 218332_at | BEX2; HBEX2; | 68533248 | 1.77 | 1.37 | 1.11 | 0.93 | 0.69 | 1.01 | down | down | 148 |
| | HGR74-h | | | | | | | | | | |
| 217370_x_at | | 861473 | 0.51 | 0.62 | 0.94 | 1.16 | 1.11 | 0.99 | up | up | 149 |
| 210665_at | EPI; TFI; | 4103170 | 0.56 | 1.05 | 1.08 | 1.09 | 1.17 | 0.99 | up | up | 150 |
| | LACI | | | | | | | | | | |
| 209006_s_at | NPD014; | 12005626 | 1.82 | 1.21 | 0.93 | 1.59 | 0.91 | 0.98 | down | down | 151 |
| | DJ465N24.2.1; | | | | | | | | | | |
| | RP3-465N24.4 | | | | | | | | | | |
| 206291_at | NN; NT; NT/N; | 31563516 | 2.15 | 1.25 | 1.12 | 1.31 | 1.04 | 0.98 | down | down | 152 |
| | NTS1; NMN- | | | | | | | | | | |
| | 125 | | | | | | | | | | |
| 204867_at | P35; GFRP; | 6382072 | 0.48 | 0.68 | 1.07 | 0.91 | 1.16 | 0.98 | up | up | 153 |
| | HsT16933; | | | | | | | | | | |
| | MGC138467; | | | | | | | | | | |
| | MGC138469 | | | | | | | | | | |
| 221957_at | PDK3 | 12356842 | 0.60 | 0.77 | 1.22 | 1.09 | 1.01 | 0.97 | up | up | 154 |
| 204865_at | Car3; | 6996001 | 0.19 | 0.37 | 0.78 | 0.65 | 1.17 | 0.97 | up | up | 155 |
| | CAIII | | | | | | | | | | |
| 200799_at | HSP72; | 26787973 | 5.33 | 1.93 | 0.84 | 1.09 | 1.03 | 0.97 | down | down | 156 |
| | HSPA1; | | | | | | | | | | |
| | HSPA1B; | | | | | | | | | | |
| | HSP70-1 | | | | | | | | | | |
| 211506_s_at | IL8 | 12641914 | 4.89 | 1.70 | 1.44 | 0.87 | 1.07 | 0.97 | down | down | 157 |
| 219208_at | VIT1; FBX11; | 30089925 | 0.54 | 0.95 | 1.10 | 0.93 | 0.89 | 0.96 | up | up | 158 |
| | PRMT9; | | | | | | | | | | |
| | FLJ12673; | | | | | | | | | | |
| | MGC44383; | | | | | | | | | | |
| | UG063H01 | | | | | | | | | | |
| 209189_at | c-fos | 33872858 | 4.78 | 1.00 | 0.95 | 0.95 | 1.02 | 0.96 | down | down | 159 |
| AFFX- | | 337376 | 1.12 | 0.40 | 1.09 | 1.09 | 1.62 | 0.96 | down | down | 160 |
| HUMRGE/M10098_5_at | | | | | | | | | | | |
| 213850_s_at | POLQ | 5812209 | 1.53 | 1.04 | 0.76 | 1.03 | 1.09 | 0.95 | down | down | 161 |
| 205067_at | IL-1; IL1F2; IL1- | 27894305 | 1.97 | 0.92 | 1.18 | 1.22 | 0.94 | 0.95 | down | down | 162 |
| | BETA | | | | | | | | | | |
| 212193_s_at | LARP1 | 10330305 | 1.52 | 1.01 | 0.70 | 1.11 | 1.10 | 0.95 | down | down | 163 |
| 205148_s_at | CLCN4 | 1578556 | 0.52 | 0.92 | 1.08 | 0.79 | 0.82 | 0.94 | up | up | 164 |
| 212107_s_at | DHX9 | 9804734 | 1.46 | 1.03 | 0.72 | 0.90 | 1.06 | 0.94 | down | down | 165 |
| 202793_at | C3F; OACT5; | 42542393 | 0.63 | 1.03 | 0.91 | 1.05 | 1.27 | 0.93 | up | up | 166 |
| | nessy | | | | | | | | | | |
| 205883_at | PLZF; | 66932930 | 1.95 | 0.96 | 1.17 | 1.28 | 0.99 | 0.92 | down | down | 167 |
| | ZNF145 | | | | | | | | | | |
| 214963_at | NUP160 | 10439023 | 0.50 | 0.84 | 1.11 | 0.85 | 0.96 | 0.92 | up | up | 168 |
| 212225_at | CSH1 | 45653459 | 2.01 | 1.21 | 0.93 | 1.28 | 0.92 | 0.91 | down | down | 169 |
| 207186_s_at | BPTF; FAC1; | 119395732 | 1.89 | 0.98 | 1.24 | 1.09 | 0.94 | 0.91 | down | down | 170 |
| | NURF301 | | | | | | | | | | |
| 206093_x_at | | 6005907 | 1.73 | 1.40 | 0.89 | 0.94 | 0.78 | 0.90 | down | down | 171 |
| 219494_at | FSBP | 20143928 | 1.89 | 1.02 | 0.93 | 0.87 | 0.91 | 0.90 | down | down | 172 |
| 215203_at | BSG | 6973770 | 0.61 | 0.86 | 1.34 | 0.91 | 0.96 | 0.90 | up | up | 173 |
| 204621_s_at | CSK | 5673966 | 1.89 | 0.87 | 1.14 | 1.19 | 1.04 | 0.90 | down | down | 174 |
| 200800_s_at | HSP72; | 26787973 | 4.87 | 1.60 | 0.87 | 1.09 | 1.04 | 0.89 | down | down | 175 |
| | HSPA1; | | | | | | | | | | |
| | HSPA1B; | | | | | | | | | | |
| | HSP70-1 | | | | | | | | | | |
| 219966_x_at | SMAR1; | 109698607 | 1.78 | 1.15 | 1.08 | 0.96 | 0.88 | 0.87 | down | down | 176 |
| | SMARBP1; | | | | | | | | | | |
| | FLJ10177; | | | | | | | | | | |
| | FLJ20538; | | | | | | | | | | |
| | DKFZp761H172 | | | | | | | | | | |
| 202628_s_at | PAI; PAI1; PAI- | 10835158 | 1.83 | 1.15 | 0.99 | 1.10 | 0.92 | 0.87 | down | down | 177 |
| | 1; PLANH1 | | | | | | | | | | |
| 207629_s_at | GEF; P40; | 15011973 | 1.77 | 1.04 | 0.75 | 1.31 | 1.00 | 0.86 | down | down | 178 |
| | GEFH1; LFP40; | | | | | | | | | | |
| | GEF-H1; | | | | | | | | | | |
| | KIAA0651; | | | | | | | | | | |
| | DKFZp547L106; | | | | | | | | | | |
| | DKFZp547P1516 | | | | | | | | | | |
| 201739_at | SGK1 | 25168262 | 2.37 | 1.26 | 0.93 | 0.90 | 0.76 | 0.85 | down | down | 179 |
| 205039_s_at | IK1; LYF1; hlk- | 146261998 | 1.56 | 0.96 | 0.68 | 0.84 | 0.88 | 0.85 | down | down | 180 |
| | 1; IKAROS; | | | | | | | | | | |
| | PRO0758; | | | | | | | | | | |
| | ZNFN1A1; | | | | | | | | | | |
| | Hs.54452 | | | | | | | | | | |
| 201694_s_at | TIS8; AT225; | 31317226 | 1.65 | 0.80 | 1.33 | 0.92 | 1.00 | 0.85 | down | down | 181 |
| | G0S30; NGFI- | | | | | | | | | | |
| | A; ZNF225; | | | | | | | | | | |
| | KROX-24; ZIF- | | | | | | | | | | |
| | 268 | | | | | | | | | | |
| 204318_s_at | B99 | 51317385 | 1.69 | 1.11 | 0.86 | 1.17 | 0.93 | 0.84 | down | down | 182 |
| 214805_at | CSH1 | 1710239 | 3.00 | 0.84 | 0.97 | 1.01 | 0.95 | 0.84 | down | down | 183 |
| 209007_s_at | NPD014; | 12006038 | 1.98 | 1.37 | 1.09 | 1.11 | 0.90 | 0.84 | down | down | 184 |
| | DJ465N24.2.1; | | | | | | | | | | |
| | RP3-465N24.4 | | | | | | | | | | |
| 212845_at | SMG; SMGA; | 5689442 | 0.47 | 1.18 | 1.10 | 0.82 | 1.17 | 0.84 | up | up | 185 |
| | SAMD4; | | | | | | | | | | |
| | Smaug; | | | | | | | | | | |
| | Smaug1; | | | | | | | | | | |
| | KIAA1053; | | | | | | | | | | |
| | DKFZp434H0350 | | | | | | | | | | |
| 200884_at | B-CK; | 34335231 | 0.43 | 0.66 | 1.10 | 0.82 | 0.94 | 0.84 | up | up | 186 |
| | CKBB | | | | | | | | | | |
| 222040_at | HNRPA1 | 3665816 | 2.50 | 0.97 | 1.16 | 1.36 | 0.70 | 0.83 | down | down | 187 |
| 210172_at | ZFM1; ZNF162; | 785998 | 1.54 | 1.00 | 0.85 | 1.11 | 0.73 | 0.82 | down | down | 188 |
| | D11S636 | | | | | | | | | | |
| 204881_s_at | GCS | 4507810 | 0.55 | 0.58 | 0.80 | 0.88 | 1.10 | 0.82 | up | up | 189 |
| 211998_at | H3F3B | 6142559 | 2.20 | 0.98 | 1.23 | 0.76 | 0.72 | 0.82 | down | down | 190 |
| 213998_s_at | DDX17 | 6462567 | 1.50 | 0.74 | 1.01 | 1.00 | 0.88 | 0.82 | down | down | 191 |
| 220319_s_at | MIR | 38788242 | 0.65 | 1.43 | 0.92 | 1.21 | 1.01 | 0.81 | up | up | 192 |
| 220969_s_at | | 13569855 | 0.58 | 0.87 | 1.39 | 0.93 | 0.83 | 0.81 | up | up | 193 |
| 209324_s_at | RGS16 | 11251810 | 1.76 | 1.19 | 0.92 | 1.41 | 0.83 | 0.80 | down | down | 194 |
| 209146_at | DESP4; | 10722273 | 1.86 | 1.18 | 0.99 | 0.87 | 1.00 | 0.80 | down | down | 195 |
| | ERG25; | | | | | | | | | | |
| | MGC104344 | | | | | | | | | | |
| 213213_at | | 8979786 | 1.58 | 1.13 | 1.15 | 1.19 | 0.66 | 0.79 | down | down | 196 |
| 207746_at | POLQ | 7662545 | 0.64 | 1.28 | 1.41 | 0.91 | 0.74 | 0.79 | up | up | 197 |
| 219676_at | ZNF392; | 13376833 | 0.48 | 0.80 | 1.09 | 0.91 | 1.15 | 0.79 | up | up | 198 |
| | ZSCAN16; | | | | | | | | | | |
| | FLJ22191; | | | | | | | | | | |
| | dJ265C24.3 | | | | | | | | | | |
| 203665_at | HO-1; | 4504436 | 0.36 | 1.08 | 0.96 | 1.18 | 1.13 | 0.79 | up | up | 199 |
| | bK286B10 | | | | | | | | | | |
| 216333_x_at | XB; TNX; XBS; | 183069 | 1.79 | 1.20 | 0.78 | 1.07 | 0.93 | 0.79 | down | down | 200 |
| | HXBL; TENX; | | | | | | | | | | |
| | TNXB1; | | | | | | | | | | |
| | TNXB2; TNXBS | | | | | | | | | | |
| 215269_at | KRT14 | 5658502 | 0.70 | 1.09 | 1.56 | 1.17 | 0.81 | 0.79 | up | up | 201 |
| 212777_at | GF1; HGF; | 306777 | 2.19 | 1.32 | 1.01 | 1.22 | 1.16 | 0.78 | down | down | 202 |
| | GGF1; GINGF | | | | | | | | | | |
| 210524_x_at | | 6683748 | 0.69 | 0.75 | 1.51 | 0.82 | 0.94 | 0.78 | up | up | 203 |
| 214483_s_at | HSU52521; | 4761515 | 1.36 | 0.97 | 0.63 | 0.94 | 0.97 | 0.78 | down | down | 204 |
| | MGC117369 | | | | | | | | | | |
| 203481_at | RPL28 | 4739881 | 1.56 | 0.99 | 1.00 | 0.91 | 1.40 | 0.78 | down | down | 205 |
| 204950_at | DACAR; | 7662403 | 1.90 | 0.92 | 1.21 | 0.95 | 1.00 | 0.77 | down | down | 206 |
| | NDPP1; | | | | | | | | | | |
| | TUCAN; | | | | | | | | | | |
| | CARDINAL; | | | | | | | | | | |
| | KIAA0955; | | | | | | | | | | |
| | MGC57162 | | | | | | | | | | |
| 218592_s_at | CECR5 | 51093854 | 1.77 | 1.46 | 1.07 | 1.02 | 0.81 | 0.77 | down | down | 207 |
| 218882_s_at | FLJ12796 | 5803220 | 1.55 | 1.08 | 0.92 | 1.00 | 1.06 | 0.76 | down | down | 208 |
| 221760_at | MAN1A1 | 13040709 | 1.92 | 1.72 | 1.11 | 1.14 | 0.80 | 0.76 | down | down | 209 |
| 213451_x_at | TNXB | 8361667 | 1.93 | 1.28 | 0.80 | 0.97 | 0.85 | 0.76 | down | down | 210 |
| 201041_s_at | HVH1; CL100; | 7108342 | 1.61 | 1.05 | 1.00 | 0.99 | 0.76 | 0.76 | down | down | 211 |
| | MKP-1; | | | | | | | | | | |
| | PTPN10 | | | | | | | | | | |
| 211992_at | WNK1 | 4290360 | 1.55 | 1.44 | 0.93 | 1.04 | 0.84 | 0.75 | down | down | 212 |
| 209933_s_at | IRC1; IRC2; | 4103065 | 1.50 | 1.06 | 1.36 | 1.40 | 0.84 | 0.75 | down | down | 213 |
| | IRp60; IGSF12; | | | | | | | | | | |
| | CMRF35H; | | | | | | | | | | |
| | CMRF-35H; | | | | | | | | | | |
| | CMRF35H9; | | | | | | | | | | |
| | CMRF-35-H9 | | | | | | | | | | |
| 221290_s_at | MUM1 | 7706014 | 1.52 | 1.09 | 1.03 | 1.07 | 0.86 | 0.74 | down | down | 214 |
| 214870_x_at | | 2951945 | 1.47 | 1.06 | 1.06 | 1.13 | 0.87 | 0.73 | down | down | 215 |
| 204506_at | CACNA1E | 45745444 | 1.44 | 0.99 | 1.07 | 0.94 | 0.97 | 0.72 | down | down | 216 |
| 211993_at | WNK1 | 5235021 | 1.61 | 0.97 | 1.12 | 1.04 | 0.79 | 0.71 | down | down | 217 |
| 211559_s_at | SCOTIN | 1236234 | 0.50 | 1.11 | 0.76 | 1.08 | 0.72 | 0.71 | up | up | 218 |
| 214157_at | GNAS | 2053970 | 0.49 | 0.76 | 1.11 | 0.79 | 0.70 | 0.71 | up | up | 219 |
| 204709_s_at | CHO1; KNSL5; | 20143965 | 1.46 | 1.06 | 0.89 | 1.20 | 1.03 | 0.70 | down | down | 220 |
| | MKLP1; MKLP-1 | | | | | | | | | | |
| 202581_at | HSP70-2 | 26787974 | 3.34 | 1.24 | 0.86 | 1.03 | 0.82 | 0.69 | down | down | 221 |
| 214881_s_at | UBF; | 28970 | 1.44 | 0.98 | 0.74 | 1.39 | 0.83 | 0.68 | down | down | 222 |
| | NOR-90 | | | | | | | | | | |
| 215645_at | | 6690149 | 0.65 | 1.06 | 1.23 | 1.38 | 0.76 | 0.68 | up | up | 223 |
| 208961_s_at | GBF; ZF9; | 3582142 | 1.38 | 0.99 | 1.00 | 1.30 | 1.00 | 0.67 | down | down | 224 |
| | BCD1; CPBP; | | | | | | | | | | |
| | PAC1; ST12; | | | | | | | | | | |
| | COPEB; | | | | | | | | | | |
| | DKFZp686N0199 | | | | | | | | | | |
| 217165_x_at | MT1; | 187540 | 0.44 | 0.51 | 0.97 | 0.79 | 0.91 | 0.67 | up | up | 225 |
| | MGC32732 | | | | | | | | | | |
| 204507_s_at | CNB; CNB1; | 45238847 | 2.13 | 0.98 | 0.87 | 1.13 | 0.95 | 0.66 | down | down | 226 |
| | CALNB1 | | | | | | | | | | |
| 209645_s_at | ALDH5; | 25777729 | 1.29 | 1.06 | 0.61 | 0.96 | 1.04 | 0.66 | down | down | 227 |
| | ALDHX; | | | | | | | | | | |
| | MGC2230 | | | | | | | | | | |
| 211527_x_at | VPF; VEGFA; | 340300 | 1.38 | 1.09 | 0.83 | 1.09 | 1.02 | 0.65 | down | down | 228 |
| | MGC70609 | | | | | | | | | | |
| 213629_x_at | MT1F | 11160133 | 0.55 | 0.38 | 1.17 | 0.79 | 1.13 | 0.65 | up | up | 229 |
| 34031_i_at | CAM; | 2832225 | 1.43 | 1.36 | 0.75 | 0.99 | 0.91 | 0.64 | down | down | 230 |
| | CCM1 | | | | | | | | | | |
| 206056_x_at | LSN; CD43; | 36455 | 1.29 | 0.85 | 1.11 | 1.06 | 0.76 | 0.64 | down | down | 231 |
| | GPL115 | | | | | | | | | | |
| 214657_s_at | CFB | 10995516 | 1.80 | 1.22 | 0.76 | 1.27 | 0.73 | 0.63 | down | down | 232 |
| 218507_at | HIG2; | 142381225 | 1.30 | 1.05 | 1.09 | 0.83 | 1.00 | 0.62 | down | down | 233 |
| | FLJ21076; | | | | | | | | | | |
| | MGC138388 | | | | | | | | | | |
| 209921_at | xCT; | 13516845 | 0.50 | 1.25 | 1.29 | 0.89 | 0.78 | 0.61 | up | up | 234 |
| | CCBR1 | | | | | | | | | | |
| 212272_at | LPIN1 | 2883245 | 1.31 | 1.45 | 1.04 | 1.03 | 1.21 | 0.61 | down | down | 235 |
| 213387_at | KIAA1240; | 6330790 | 1.51 | 1.02 | 1.23 | 0.83 | 0.87 | 0.60 | down | down | 236 |
| | MGC88424 | | | | | | | | | | |
| 205260_s_at | ACYPE | 45243543 | 1.20 | 0.96 | 1.03 | 0.96 | 0.84 | 0.60 | down | down | 237 |
| 214295_at | NFIA | 6117000 | 0.47 | 0.81 | 1.80 | 0.89 | 0.78 | 0.59 | up | up | 238 |
| 214007_s_at | | 7457569 | 1.38 | 1.01 | 0.55 | 0.72 | 0.77 | 0.58 | down | down | 239 |
| 205632_s_at | MSS4; | 115529452 | 1.29 | 1.36 | 0.96 | 0.94 | 0.84 | 0.58 | down | down | 240 |
| | STM7 | | | | | | | | | | |
| 208960_s_at | KLF6 | 10035976 | 1.33 | 1.01 | 0.83 | 1.15 | 1.01 | 0.58 | down | down | 241 |
| 210513_s_at | VPF; VEGFA; | 5901560 | 1.39 | 0.99 | 0.79 | 0.90 | 0.98 | 0.58 | down | down | 242 |
| | MGC70609 | | | | | | | | | | |
| 212501_at | | 46231593 | 1.22 | 1.04 | 1.07 | 0.94 | 0.74 | 0.57 | down | down | 243 |
| 219498_s_at | EVI9; CTIP1; | 20336306 | 1.74 | 1.17 | 0.98 | 0.76 | 0.65 | 0.57 | down | down | 244 |
| | BCL11A-L; | | | | | | | | | | |
| | BCL11A-S; | | | | | | | | | | |
| | FLJ10173; | | | | | | | | | | |
| | FLJ34997; | | | | | | | | | | |
| | KIAA1809; | | | | | | | | | | |
| | BCL11A-XL | | | | | | | | | | |
| 39248_at | HBA2 | 1231892 | 1.61 | 1.37 | 0.98 | 0.93 | 0.67 | 0.57 | down | down | 245 |
| 201531_at | TTP; G0S24; | 141802261 | 1.70 | 1.08 | 1.02 | 1.21 | 0.95 | 0.57 | down | down | 246 |
| | G0S24; TIS11; | | | | | | | | | | |
| | NUP475; | | | | | | | | | | |
| | RNF162A | | | | | | | | | | |
| 202934_at | HK2 | 5177228 | 1.29 | 1.16 | 0.99 | 1.03 | 0.86 | 0.56 | down | down | 247 |
| 204243_at | Zn-15L; | 142368487 | 1.16 | 1.24 | 0.89 | 1.00 | 1.00 | 0.56 | down | down | 248 |
| | ZNF292L; | | | | | | | | | | |
| | MGC142226 | | | | | | | | | | |
| 221245_s_at | | 13540591 | 0.56 | 0.70 | 1.13 | 0.79 | 0.74 | 0.56 | up | down | 249 |
| 35776_at | ITSN; SH3D1A; | 3859852 | 1.20 | 1.04 | 1.10 | 0.87 | 0.74 | 0.55 | down | down | 250 |
| | SH3P17; | | | | | | | | | | |
| | MGC134948; | | | | | | | | | | |
| | MGC134949 | | | | | | | | | | |
| 216336_x_at | | 6729581 | 0.35 | 0.45 | 1.21 | 0.82 | 0.89 | 0.54 | up | up | 251 |
| 201473_at | JUNB | 44921611 | 1.24 | 0.84 | 1.01 | 0.96 | 0.72 | 0.54 | down | down | 252 |
| 213434_at | STX2 | 1102896 | 1.31 | 0.99 | 0.84 | 0.86 | 0.82 | 0.53 | down | down | 253 |
| 203249_at | KIAA0388 | 2224716 | 1.16 | 0.82 | 0.77 | 1.10 | 0.70 | 0.53 | down | down | 254 |
| 203126_at | CDK6 | 7657235 | 1.63 | 0.86 | 0.79 | 1.08 | 0.65 | 0.52 | down | down | 255 |
| 205967_at | H4/g; H4FG; | 21071024 | 1.16 | 0.92 | 0.96 | 0.94 | 0.74 | 0.52 | down | down | 256 |
| | dJ221C16.1 | | | | | | | | | | |
| 210347_s_at | EVI9; CTIP1; | 12150277 | 1.29 | 0.97 | 0.92 | 0.86 | 0.82 | 0.51 | down | down | 257 |
| | BCL11A-L; | | | | | | | | | | |
| | BCL11A-S; | | | | | | | | | | |
| | FLJ10173; | | | | | | | | | | |
| | FLJ34997; | | | | | | | | | | |
| | KIAA1809; | | | | | | | | | | |
| | BCL11A-XL | | | | | | | | | | |
| 218136_s_at | | 28466986 | 1.06 | 0.99 | 0.86 | 1.07 | 0.70 | 0.50 | down | down | 258 |
| 211560_s_at | | 11493529 | 1.11 | 1.03 | 0.85 | 0.95 | 0.78 | 0.50 | down | down | 259 |
| 209112_at | KIP1; CDKN4; | 12805034 | 1.00 | 0.95 | 0.98 | 0.92 | 0.72 | 0.50 | down | down | 260 |
| | P27KIP1 | | | | | | | | | | |
| 212907_at | SLC30A1 | 5769332 | 0.49 | 1.01 | 1.02 | 0.82 | 0.63 | 0.50 | up | up | 261 |
| 201732_s_at | CLC3; | 2599547 | 1.11 | 0.98 | 0.83 | 0.89 | 0.88 | 0.49 | down | down | 262 |
| | CIC-3 | | | | | | | | | | |
| 219878_s_at | BTEB3; FKLF2; | 37693994 | 1.13 | 0.74 | 0.89 | 0.87 | 0.79 | 0.49 | down | down | 263 |
| | NSLP1; | | | | | | | | | | |
| | RFLAT1; | | | | | | | | | | |
| | RFLAT-1 | | | | | | | | | | |
| 218274_s_at | ZNF744; | 109150424 | 1.02 | 1.03 | 1.02 | 1.06 | 0.77 | 0.49 | up | down | 264 |
| | FLJ10415 | | | | | | | | | | |
| 203911_at | RAP1GA1; | 4506414 | 1.11 | 1.01 | 1.01 | 0.99 | 0.89 | 0.47 | down | down | 265 |
| | KIAA0474; | | | | | | | | | | |
| | rap1GAPII | | | | | | | | | | |
| 203946_s_at | ARG2 | 1763757 | 1.08 | 1.32 | 0.99 | 1.10 | 0.84 | 0.47 | down | down | 266 |
| 212185_x_at | MT2 | 31543214 | 0.39 | 0.51 | 1.10 | 0.77 | 1.00 | 0.47 | up | down | 267 |
| 205842_s_at | JAK2 | 3236321 | 1.32 | 1.25 | 0.76 | 0.87 | 0.74 | 0.46 | down | down | 268 |
| 203845_at | PCAF | 55949646 | 1.00 | 1.11 | 0.91 | 0.80 | 0.64 | 0.46 | down | down | 269 |
| 211456_x_at | LOC645745 | 13310411 | 0.41 | 0.51 | 1.10 | 0.79 | 1.04 | 0.46 | up | up | 270 |
| 219630_at | DD96; SPAP; | 41152089 | 1.00 | 0.98 | 0.96 | 1.06 | 1.01 | 0.46 | down | down | 271 |
| | MAP17; RP1- | | | | | | | | | | |
| | 18D14.5 | | | | | | | | | | |
| 205896_at | OCTN1; | 24497489 | 1.13 | 1.19 | 1.12 | 1.07 | 0.66 | 0.45 | down | down | 272 |
| | MGC34546; | | | | | | | | | | |
| | MGC40524 | | | | | | | | | | |
| 207459_x_at | SS; GPB; MNS; | 75905814 | 0.92 | 0.99 | 0.88 | 0.83 | 0.62 | 0.45 | down | down | 273 |
| | GYPA; | | | | | | | | | | |
| | CD235b; | | | | | | | | | | |
| | GPB.NY; | | | | | | | | | | |
| | GYPHe.NY | | | | | | | | | | |
| 221778_at | KIAA1718 | 8905200 | 1.14 | 0.99 | 0.90 | 1.00 | 0.68 | 0.45 | down | down | 274 |
| 204326_x_at | | 28976166 | 0.35 | 0.49 | 1.20 | 0.83 | 1.08 | 0.44 | up | up | 275 |
| 208581_x_at | MT1; MT- | 31543213 | 0.35 | 0.49 | 1.08 | 0.77 | 0.92 | 0.44 | up | up | 276 |
| | 1I | | | | | | | | | | |
| 201170_s_at | DEC1; | 4503298 | 1.48 | 0.71 | 0.97 | 0.78 | 0.62 | 0.43 | down | down | 277 |
| | STRA13; | | | | | | | | | | |
| | Stra14; | | | | | | | | | | |
| | SHARP-2 | | | | | | | | | | |
| 212859_x_at | MT1E | 11111447 | 0.26 | 0.35 | 1.32 | 0.77 | 0.81 | 0.43 | up | up | 278 |
| 219497_s_at | EVI9; CTIP1; | 20336304 | 1.67 | 1.22 | 0.96 | 0.69 | 0.70 | 0.43 | down | down | 279 |
| | BCL11A-L; | | | | | | | | | | |
| | BCL11A-S; | | | | | | | | | | |
| | FLJ10173; | | | | | | | | | | |
| | FLJ34997; | | | | | | | | | | |
| | KIAA1809; | | | | | | | | | | |
| | BCL11A-XL | | | | | | | | | | |
| 206461_x_at | MT1; | 124244058 | 0.27 | 0.43 | 1.11 | 0.72 | 0.90 | 0.43 | up | up | 280 |
| | MGC70702 | | | | | | | | | | |
| 202124_s_at | ATXN1 | 10722559 | 1.01 | 1.00 | 0.97 | 0.93 | 0.70 | 0.43 | down | down | 281 |
| 206686_at | PTPRF | 37595546 | 0.50 | 0.70 | 0.87 | 0.85 | 1.03 | 0.42 | up | down | 282 |
| 214407_x_at | GYPA | 3835942 | 0.87 | 1.02 | 0.83 | 0.87 | 0.67 | 0.42 | down | down | 283 |
| 219410_at | DERP7; | 8922242 | 0.35 | 0.61 | 0.79 | 1.02 | 1.05 | 0.42 | up | up | 284 |
| | FLJ10134 | | | | | | | | | | |
| 203414_at | MMA; | 52630444 | 0.85 | 0.76 | 1.23 | 0.73 | 0.65 | 0.41 | up | down | 285 |
| | PAQR11 | | | | | | | | | | |
| 207220_at | DO; DOK1 | 61835133 | 0.90 | 1.07 | 0.96 | 0.81 | 0.68 | 0.40 | up | down | 286 |
| | CD297 | | | | | | | | | | |
| 216063_at | beta globin | 1198084 | 0.43 | 0.57 | 0.93 | 0.73 | 0.70 | 0.40 | up | down | 287 |
| | pseudogene | | | | | | | | | | |
| 207854_at | GPE; | 38373678 | 0.81 | 1.11 | 0.82 | 0.69 | 0.72 | 0.40 | up | down | 288 |
| | MNS; | | | | | | | | | | |
| | MiIX | | | | | | | | | | |
| 218489_s_at | PBGS; ALADH; | 51558761 | 1.01 | 0.83 | 0.73 | 1.06 | 0.64 | 0.40 | down | down | 289 |
| | MGC5057 | | | | | | | | | | |
| 211821_x_at | MN; GPA; | 392430 | 0.90 | 0.88 | 0.80 | 0.83 | 0.73 | 0.39 | down | down | 290 |
| | MNS; GPSAT; | | | | | | | | | | |
| | CD235a; | | | | | | | | | | |
| | GPErik; | | | | | | | | | | |
| | HGpMiV; | | | | | | | | | | |
| | HGpMiX; | | | | | | | | | | |
| | GpMilII; | | | | | | | | | | |
| | HGpMiXI; | | | | | | | | | | |
| | HGpMiIII; | | | | | | | | | | |
| | HGpSta(C) | | | | | | | | | | |
| 217678_at | SLC7A11 | 2216118 | 0.35 | 1.36 | 1.17 | 0.87 | 0.71 | 0.39 | up | up | 291 |
| 209566_at | MGC26273 | 5262661 | 0.99 | 1.06 | 0.98 | 0.90 | 0.78 | 0.39 | down | down | 292 |
| 202364_at | MXI; MAD2; | 57242781 | 0.79 | 1.06 | 0.89 | 0.85 | 0.71 | 0.39 | up | down | 293 |
| | MXD2; | | | | | | | | | | |
| | MGC43220 | | | | | | | | | | |
| 204467_s_at | PD1; NACP; | 6806896 | 1.11 | 1.03 | 0.99 | 0.91 | 0.69 | 0.38 | down | down | 294 |
| | PARK1; | | | | | | | | | | |
| | PARK4; | | | | | | | | | | |
| | MGC110988 | | | | | | | | | | |
| 204745_x_at | MT1; MT1K; | 10835229 | 0.25 | 0.36 | 1.15 | 0.69 | 0.89 | 0.36 | up | up | 295 |
| | MGC12386 | | | | | | | | | | |
| 208335_s_at | FY; Dfy; GPD; | 42822886 | 0.76 | 0.90 | 0.84 | 0.81 | 0.62 | 0.36 | up | down | 296 |
| | CCBP1; CD234 | | | | | | | | | | |
| 221748_s_at | FGFR1 | 5435035 | 1.19 | 1.05 | 1.07 | 0.97 | 0.55 | 0.35 | down | down | 297 |
| 218145_at | NIPK; SINK; | 41327717 | 0.99 | 0.92 | 1.09 | 0.96 | 0.49 | 0.34 | up | down | 298 |
| | TRB3; SKIP3; | | | | | | | | | | |
| | C20orf97 | | | | | | | | | | |
| 221920_s_at | SLC25A37 | 10038376 | 1.13 | 1.01 | 0.96 | 1.05 | 0.63 | 0.33 | down | down | 299 |
| 212543_at | ST4 | 2072424 | 0.34 | 0.63 | 0.81 | 0.66 | 0.77 | 0.33 | up | down | 300 |
| 201849_at | NIP3 | 7669480 | 0.23 | 0.60 | 1.11 | 0.81 | 0.78 | 0.32 | up | up | 301 |
| 201848_s_at | NIP3 | 558845 | 0.27 | 0.56 | 0.95 | 0.85 | 0.86 | 0.32 | up | up | 302 |
| 202887_s_at | Dig2; REDD1; | 56676369 | 0.66 | 0.61 | 1.06 | 0.88 | 0.47 | 0.30 | up | down | 303 |
| | REDD-1; | | | | | | | | | | |
| | RTP801; | | | | | | | | | | |
| | FLJ20500; | | | | | | | | | | |
| | RP11-442H21.1 | | | | | | | | | | |
| 215449_at | SLC25A30 | 3308215 | 0.81 | 0.77 | 1.05 | 0.76 | 0.67 | 0.29 | up | down | 304 |
| 208886_at | H10; H1FV; | 12652786 | 0.88 | 0.84 | 0.97 | 0.86 | 0.49 | 0.28 | up | down | 305 |
| | MGC5241 | | | | | | | | | | |
| 205592_at | K60; NAF; | 535096 | 1.00 | 1.05 | 0.96 | 0.85 | 0.53 | 0.28 | down | down | 306 |
| | GCP1; IL-8; | | | | | | | | | | |
| | LECT; LUCT; | | | | | | | | | | |
| | NAP1; 3-10C; | | | | | | | | | | |
| | CXCL8; GCP-1; | | | | | | | | | | |
| | LYNAP; | | | | | | | | | | |
| | MDNCF; | | | | | | | | | | |
| | MONAP; NAP- | | | | | | | | | | |
| | 1; SCYB8; | | | | | | | | | | |
| | TSG-1; AMCF-I; | | | | | | | | | | |
| | b-ENAP | | | | | | | | | | |

We have identified the top genes that are differentially regulated in B19 infected CD36+ cells at 6 hours post infection and 48 hours post infection as an example to demonstrate the early and late gene expressions. Genes differentially expressed in viral infected cells can be utilized in diagnostic kits and for detection of B19 infected cells. The gene expression profile of one or more genes differentially regulated can be used to identify virus infected cells. Such genes can be selected from those provided in Table 16 or Table 17.

TABLE 16
The table below shows the top gene genes differentially expressed
at timepoints 6 hours and 48 hours post-infection.

| Description | 6 hr PI |
|---|---|
| interleukin 8 | 2.225 |
| elastase 2, neutrophil | 1.831 |
| Nuclear factor I/A | 1.804 |
| myeloperoxidase | 1.792 |
| AV711904 DCA Homo sapiens cDNA clone DCAAIE08 5′, | 1.756 |
| mRNA sequence. | |
| Cytochrome P450, family 1, subfamily B, polypeptide 1 | 1.66 |
| ATP synthase, H+ transporting, mitochondrial F1 | 1.61 |
| complex, gamma polypeptide 1 | |
| interferon-induced protein 44 | 1.598 |
| immediate early response 3 | 1.583 |
| interferon-induced protein with tetratricopeptide | 1.569 |
| repeats 1 | |
| Description | 48 hr PI |
| AV711904 DCA Homo sapiens cDNA clone DCAAIE08 5′, | 5.34 |
| mRNA sequence. | |
| Charcot-Leyden crystal protein | 4.35 |
| tachykinin 3 (neuromedin K, neurokinin beta) | 3.917 |
| cytochrome P450, family 1, subfamily B, polypeptide 1 | 3.833 |
| elastase 2, neutrophil | 3.638 |
| myeloperoxidase | 3.21 |
| myeloperoxidase | 3.124 |
| Cytochrome P450, family 1, subfamily B, polypeptide 1 | 3.121 |
| carboxypeptidase A3 (mast cell) | 2.952 |
| actin, alpha 2, smooth muscle, aorta | 2.944 |

TABLE 17

| | Gene | GI | 0 | 6 |
|---|---|---|---|---|
| 202859_x_at | | NM_000584 | 5.65 | 2.225 |
| 206871_at | | NM_001972 | 8.71 | 1.831 |
| 214295_at | NFIA | AW129056 | 0.47 | 1.804 |
| 203949_at | IL6 | NM_000250 | 6.12 | 1.792 |
| 213975_s_at | | AV711904 | 12.77 | 1.756 |
| 202436_s_at | CYP1B1 | AU144855 | 1.23 | 1.66 |
| 214132_at | ATP5C1 | BG232034 | 0.73 | 1.61 |
| 214453_s_at | p44; | NM_006417 | 0.96 | 1.598 |
| | MTAP44 | | | |
| 201631_s_at | | NM_003897 | 2.23 | 1.583 |
| 203153_at | | NM_001548 | 0.89 | 1.569 |

| Probe set | Gene | GI | 0 | 48 |
|---|---|---|---|---|
| 213975_s_at | | AV711904 | 12.77 | 5.34 |
| 206207_at | | NM_001828 | 2.84 | 4.35 |
| 219992_at | | NM_013251 | 1.04 | 3.92 |
| 202437_s_at | CP1B; | NM_000104 | 0.78 | 3.83 |
| | GLC3A | | | |
| 206871_at | | NM_001972 | 8.71 | 3.64 |
| 203949_at | IL6 | NM_000250 | 6.12 | 3.21 |
| 203948_s_at | IL6 | J02694 | 6.74 | 3.12 |

One or more of these genes are useful to identify parvovirus B19 infected cells even at early stages of infection.

It should be noted that, as used in this specification and the appended claims, the singular forms “a”, “an” and “the” include plural referents unless the content clearly dictates otherwise. It should also be noted that the term “or” is generally employed in its sense including “and/or” unless the content clearly dictates otherwise.

All publications and patent applications in this specification are indicative of the level of ordinary skill in the art to which this disclosure pertains. All publications and patent applications are herein incorporated by reference to the same extent as if each individual publication or patent application was specifically and individually indicated by reference.

The disclosure has been described with reference to various specific and preferred embodiments and techniques. However, it should be understood that many variations and modifications may be made while remaining within the spirit and scope of the disclosure.

1. A method for producing parvovirus B19, comprising:
introducing a parvovirus B19 genome into a CD36⁺ erythroid progenitor cell and culturing the cell under conditions to provide for replication of parvovirus B19 genome.

2. The method of claim 1, wherein introducing parvovirus B19 into a CD36⁺ erythroid progenitor cell comprises contacting the cells with parvovirus B19 isolated from serum.

3. The method of claim 1, wherein introducing parvovirus B19 into a CD36⁺ erythroid progenitor cell comprises introducing a vector comprising an infectious clone of parvovirus B19 into the cells.

4. The method of claim 3, wherein the infectious clone comprises a nucleic acid sequence having at least 90% nucleic acid identity to SEQ ID NO:1 or SEQ ID NO:2.

5. The method of claim 1, further comprising producing CD36⁺ erythroid progenitor cell comprising culturing hematopoietic stem cells in expansion media comprising stem cell factor (SCF), interleukin 3 (IL-3), and erythropoietin under conditions that allow for expansion and differentiation of the cells to a population of cells having at least 25% CD36⁺ cells.

6. The method of claim 5, wherein the expansion media comprises 10⁻⁶ M IL-3, 100 ng/ml recombinant human SCF, and 3 IU/ml recombinant human erythropoietin.

7. The method of claim 5, wherein the expansion media further comprises hydrocortisone.

8. The method of claim 5, wherein the hematopoietic stem cells are cultured in the expansion media for about 4 days under conditions that allow for expansion and differentiation of the cells, diluted in expansion media, and the diluted cells are cultured for about an additional 4 days under conditions that allow for expansion and differentiation of the cells.

9. The method of claim 1, wherein the CD36⁺ erythroid progenitor cells are CD36⁺, CD44⁺, CD235a⁺, CD34⁻, CD19⁻, CD10⁻, CD4⁻, CD3⁻, and CD2⁻.

10. The method of claim 5, wherein the hematopoietic stem cells have CD34, CD133, or both on the cell surface.

11. The method of claim 1, wherein the CD36⁺ erythroid progenitor cells are non-enucleated.

12. The method of claim 1, wherein the CD36⁺ erythroid progenitor cells comprise at least one of the following characteristics selected from the group consisting of: non-enucleated, CD44⁺, CD34⁻, CD19⁻, CD10⁻, CD4⁻, CD3⁻, CD2⁻, hemoglobin, globoside, and combinations thereof.

13. The method of claim 5, wherein the population of CD36⁺ erythroid progenitor cells comprise at least 25% to 100% CD36+ cells.

14. The method of claim 5, wherein the population of CD36⁺ erythroid progenitor cells comprise at least 25% CD36+ cells and 25% globoside positive cells.

15. The method of claim 1, further comprising detecting reproduction of the parvovirus B19 viral genome, transcripts, or viral protein.

16. The method of claim 15, wherein detecting reproduction of the parvovirus B19 viral genome comprises detecting B19 DNA, spliced capsid transcripts, unspliced capsid or NS protein transcripts, or B19 capsid protein in the infected cells.

17. The method of claim 15, wherein the B19 capsid protein is detected by binding to a specific antibody for B19 capsid protein.

18. The method of claim 15, wherein the B19 transcripts are detected using RT-PCR or by qRT-PCR.

19. The method of claim 15, wherein detecting reproduction of the parvovirus B19 viral genome comprises detecting B19 viral DNA in the cell.

20. The method of claim 1, wherein replication of the parvovirus B19 viral genome in the CD36⁺ erythroid progenitor cells is greater than replication of the viral genome in UT7/Epo-S1 cells.

21. The method of claim 20, wherein replication of the parvovirus B19 viral genome in the CD36⁺ erythroid progenitor cells is at least 10 fold greater compared to UT7/Epo-S1 cells.

22. The method of claim 20, wherein replication of the parvovirus B19 viral genome in the CD36⁺ erythroid progenitor cells is at least 100 fold greater compared to UT7/Epo-S1 cells.

23. The method of claim 20, wherein replication of the parvovirus B19 viral genome in the CD36⁺ erythroid progenitor cells is at least 500 fold greater compared to UT7/Epo-S1 cells.

24. The method of claim 1, wherein the replicated parvovirus B19 is infectious.

25. The method of claim 1, further comprising detecting reproduction of the parvovirus B19 comprising contacting permissive cells with supernatant from the infected CD36⁺ erythroid progenitor cells and analyzing the contacted permissive cells for B19 spliced capsid transcripts or other B19 transcripts or B19 capsid protein, wherein detection of B19 transcripts or other B19 transcripts or B19 capsid protein indicates the parvovirus B19 is infectious.

26. The method of claim 25, wherein the erythroid progenitor cells are CD36⁺, CD44⁺, CD235a⁺, CD34⁻, CD19⁻, CD10⁻, CD4⁻, CD3⁻, and CD2⁻.

27. A cell population comprising erythroid progenitor cells, wherein at least 25% to 100% of the erythroid progenitor cells are CD36+ and globoside+cells, and less than 70% of the cell population are CD33⁺.

28. CD36+ erythroid progenitor cells produced by a method comprising:
culturing hematopoietic stem cells in expansion media comprising stem cell factor (SCF), interleukin 3 (IL-3), and erythropoietin under conditions that allow for expansion and differentiation of the cells to a population of cells having at least 25% CD36⁺ cells.

29. The erythroid progenitor cells of claim 28, wherein the expansion media comprises 10⁻⁶ M IL-3, 100 ng/ml recombinant human SCF, and 3 IU/ml recombinant human erythropoietin.

30. The erythroid progenitor cells of claim 28, wherein the expansion media further comprises hydrocortisone.

31. The erythroid progenitor cells of any of claims claim 1, wherein the hematopoietic stem cells are cultured in the expansion media for about 4 days under conditions that allow for expansion and differentiation of the cells, diluted in expansion media, and the diluted cells are cultured for about an additional 4 days under conditions that allow for expansion and differentiation of the cells.

32. The cell population or erythroid progenitor cells of claim 27, wherein the CD36⁺ erythroid progenitor cells are CD36⁺, CD44⁺, CD235a⁺, CD34⁻, CD19⁻, CD10⁻, CD4⁻, CD3⁻, and CD2⁻.

33. The erythroid progenitor cells of claim 28, wherein the hematopoietic stem cells have CD34, CD133, or both on the cell surface.

34. Immortalized erythroid progenitor cells produced by a method comprising:
(a) culturing hematopoietic stem cells in expansion media under conditions that allow for expansion and differentiation of the cells to a population of at least 25% CD36⁺ cells; and
(b) immortalizing the CD36⁺ erythroid progenitor cells with a virus or viral vector.

35. The immortalized erythroid progenitor cells of claim 34, wherein (b) comprises transfecting the CD36⁺ erythroid progenitor cells with a viral vector comprising SV40 large T-antigen.

36. The immortalized erythroid progenitor cells of claim 34, wherein the viral vector comprises adenovirus or lentivirus.

37. The immortalized erythroid progenitor cells of claim 34, wherein the method further comprises culturing the hematopoietic stem cells in expansion media for about 4 days under conditions that allow for expansion and differentiation of the cells, diluting the cells in expansion media, and culturing the diluted cells for about 4 day under conditions that allow for expansion and differentiation of the cells.

38. The immortalized erythroid progenitor cells of claim 34, wherein the immortalized erythroid progenitor cells are CD36⁺, CD44⁺, CD235a⁺, CD34⁻, CD19⁻, CD10⁻, CD4⁻, CD3⁻, and CD2⁻.

39. The immortalized erythroid progenitor cells of claim 34, wherein the cells are non-enucleated.

40. The immortalized erythroid progenitor cells of claim 34, wherein the cells comprise hemoglobin and/or globoside.

41. An immortalized erythroid progenitor cell of claim 34, wherein the cell is CD36⁺, CD44⁺, CD235a⁺, CD34⁻, CD19⁻, CD10⁻, CD4⁻, CD3⁻, and CD2⁻.

42. The immortalized erythroid progenitor cell of claim 34 that can divide at least 2 to 50 times.

43. A method of detecting a parvovirus B19 infection comprising contacting the CD36⁺ erythroid progenitor cell of claim 28 with a sample; culturing the cells under conditions suitable for viral replication; and detecting the presence of the virus in the cell.

44. The method of claim 43, wherein the CD36⁺ erythroid progenitor cell are cultured in expansion media comprising stem cell factor (SCF), interleukin 3 (IL-3), and erythropoietin under conditions that allow for expansion and differentiation of the cells to a population of cells having at least 25% CD36⁺ cells.

45. The method of claim 44, wherein the expansion media comprises 10⁻⁶ M IL-3, 100 ng/ml recombinant human SCF, and 3 IU/ml recombinant human erythropoietin.

46. The method of claim 44, wherein the expansion media further comprises hydrocortisone.

47. The method of claim 43, wherein the CD36⁺ erythroid progenitor cells are CD36⁺, CD44⁺, CD235a⁺, CD34⁻, CD19⁻, CD10⁻, CD4⁻, CD3⁻, and CD2⁻.

48. The method of claim 43, wherein the CD36⁺ erythroid progenitor cells are non-enucleated.

49. The method of claim 43, wherein the CD36⁺ erythroid progenitor cells comprise at least one of the following characteristics selected from the group consisting of: non-enucleated, CD44⁺, CD34⁻, CD19⁻, CD10⁻, CD4⁻, CD3⁻, CD2⁻, hemoglobin, globoside, and combinations thereof.

50. The method of claim 43, wherein the population of CD36⁺ erythroid progenitor cells comprise at least 25% to 100% CD36+ cells.

51. The method of claim 43, wherein the population of CD36⁺ erythroid progenitor cells comprise at least 25% CD36+ cells and 25% globoside positive cells.

52. The method of claim 43, further comprising detecting reproduction of the parvovirus B19 viral genome, transcripts, or viral protein.

53. The method of claim 52, wherein detecting reproduction of the parvovirus B19 viral genome comprises detecting B19 DNA, spliced capsid transcripts, unspliced capsid or NS protein transcripts, or B19 capsid protein in the infected cells.

54. The method of claim 52, wherein the B19 capsid protein is detected by binding to a specific antibody for B19 capsid protein.

55. The method of claim 52 wherein the B19 transcripts are detected using RT-PCR or by qRT-PCR.

56. The method of claim 52, wherein detecting reproduction of the parvovirus B19 viral genome comprises detecting B19 viral DNA in the cell.

57. A method of detecting a parvovirus B19 infection comprising contacting the CD36⁺ erythroid progenitor cell of claim with a sample; culturing the cells under conditions suitable for viral replication; and detecting the gene expression profile of at least one of the genes of Table 15 and at least one parvovirus B19 viral genome, transcript, or viral protein.

58. The method of claim 58, wherein expression of at least one or all of the genes of Table 16 are detected.

59. The method of claim 57, wherein expression of the genes is detected at 6 hours post infection.

60. The method of claim 57, wherein expression of the genes is detected at 48 hours post infection.

61. The method of claim 57, wherein the gene expression is detected by an oligonucleotide that specifically binds to the polynucleotide encoding the gene.

62. A kit for detecting antibodies to parvovirus B19, comprising a composition of a CD36+ erythroid progenitor cell of claim 27, and a composition of a parvovirus B19 virus sample.

63. The kit of claim 62, wherein the composition comprises at least 10³ genomes/ml of parvovirus B19.

64. A kit for detecting or diagnosing parvovirus B19 infection, comprising a composition comprising a CD36+ erythroid progenitor cell of claim 43, and at least one oligonucleotide that specifically binds to parvovirus B19 genome or at least one viral transcript and/or an antibody that specifically binds to a viral protein.

65. A kit for detecting or diagnosing parvovirus B19 infection comprising a) a composition comprising: a CD36+ erythroid progenitor cell of claim 27; b) at least one oligonucleotide that specifically binds to parvovirus B19 genome or at least one viral transcript and/or an antibody that specifically binds to a viral protein; and c) at least one oligonucleotide that specifically binds to at least one of the genes of Table 15.

66. A microarray that comprises agents that bind 400 different genes or less including at least one or all of the genes of Table 15.

67. The microarray of claim 66, that comprises agents that bind at least one or all of the genes of Table 16.

68. The microarray of claim 66 or claim 67, that comprises agents that bind at least one or all of the genes of Table 16 and at least one or all of the parvovirus B19 transcripts.
